SEQUENCE LISTING (124 sequences)

SEQ ID NO: 1 - protein, 230 aa, synthetic construct
  REGION 1..230: note = Human IL9Ra ICD;;
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 2 - DNA, 690 bp, synthetic construct
  misc_feature 1..690: note = Human IL9Ra ICD;;
  source 1..690: mol_type = other DNA
aagctgagccccagggtgaagaggatcttctaccagaacgtgcccagccccgccatgttcttccagcccctgtacagcgtgcacaacggcaacttccagacctggatgggcgcccacggcgccggcgtgctgctgagccaggactgcgccggcaccccccagggcgccctggagccctgcgtgcaggaggccaccgccctgctgacctgcggccccgccaggccctggaagagcgtggccctggaggaggagcaggagggccccggcaccaggctgcccggcaacctgagcagcgaggacgtgctgcccgccggctgcaccgagtggagggtgcagaccctggcctacctgccccaggaggactgggcccccaccagcctgaccaggcccgccccccccgacagcgagggcagcaggagcagcagcagcagcagcagcagcaacaacaacaactactgcgccctgggctgctacggcggctggcacctgagcgccctgcccggcaacacccagagcagcggccccatccccgccctggcctgcggcctgagctgcgaccaccagggcctggagacccagcagggcgtggcctgggtgctggccggccactgccagaggcccggcctgcacgaggacctgcagggcatgctgctgcccagcgtgctgagcaaggccaggagctggaccttc

SEQ ID NO: 3 - DNA, 690 bp, synthetic construct
  misc_feature 1..690: note = Human IL9Ra ICD;;
  source 1..690: mol_type = other DNA
aagctgagccctagagtgaaaagaatcttctaccagaacgtgccttctccagccatgttcttccagcctctgtacagcgtgcacaacggcaacttccagacctggatgggcgctcacggcgccggcgttctgctgagccaggattgcgccggaacacctcaaggcgctctggaaccttgcgtgcaggaggccaccgccctgctgacctgtggccctgctcggccctggaagtccgtggccctagaggaagagcaggaaggccccgggaccagactgcctggcaacctgagcagcgaggacgtgctgcctgccggatgtactgagtggcgggtgcagaccctggcctacctgccccaggaggactgggctccaacatctctgaccagaccggcccctccagacagcgaaggcagcagatctagcagcagcagcagttcttctaataacaacaactactgtgccctcggctgttacggcggctggcacctgagcgccctccctggaaatacacagtctagcggccctatccccgccctggcttgtggactgagctgcgaccaccagggcctggaaacacagcagggcgtggcctgggtcctggccggccactgccagagacctggcctgcacgaggacctgcagggaatgctgctgcccagcgtcctgagcaaggccagaagctggaccttt

SEQ ID NO: 4 - protein, 177 aa, synthetic construct
  REGION 1..177: note = Murine IL9Ra ICD;;
KLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDSGSSDYCMLDCCEECHLSAFPGHTESPELTLAQPVALPVSSRA

SEQ ID NO: 5 - DNA, 531 bp, synthetic construct
  misc_feature 1..531: note = Murine IL9Ra ICD;;
  source 1..531: mol_type = other DNA
aagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgccccaggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcc

SEQ ID NO: 6 - DNA, 531 bp, synthetic construct
  misc_feature 1..531: note = Murine IL9Ra ICD;;
  source 1..531: mol_type = other DNA
aagctgtcacctcgccttaaacgaatcttctaccagaatatcccgtcgcctgaggctttcttccaccctctctatagtgtctaccacggagacttccaatcgtggactggtgcaaggcgggccggcccacaagccaggcaaaatggtgtgtcaacaagcagcgcaggcagtgagtcttccatctgggaagcggtcgccaccttgacttacagtccagcttgtccagtccagtttgcctgcctaaagtgggaggcaacagcccccggattccccggtctgcccggcagtgaacatgtactacctgcaggatgtttggagttagaaggccaaccttcagcatacttgcctcaggaagactgggctccactgggctctgctagaccaccccctccagactccgactcagggtcctcagactactgcatgctggactgctgtgaggagtgtcacctgagcgctttccctgggcacactgaatctccagaactgactctggcccagcccgtggccctgccagtatcaagccgagcc

SEQ ID NO: 7 - DNA, 531 bp, synthetic construct
  misc_feature 1..531: note = Murine IL9Ra ICD;;
  source 1..531: mol_type = other DNA
aagctgagccccaggctgaagaggatcttctaccagaacatccccagccccgaggccttcttccaccccctgtacagcgtgtaccacggcgacttccagagctggaccggcgccaggagggccggcccccaggccaggcagaacggcgtgagcaccagcagcgccggcagcgagagcagcatctgggaggccgtggccaccctgacctacagccccgcctgccccgtgcagttcgcctgcctgaagtgggaggccaccgcccccggcttccccggcctgcccggcagcgagcacgtgctgcccgccggctgcctggagctggagggccagcccagcgcctacctgccccaggaggactgggcccccctgggcagcgccaggccccccccccccgacagcgacagcggcagcagcgactactgcatgctggactgctgcgaggagtgccacctgagcgccttccccggccacaccgagagccccgagctgaccctggcccagcccgtggccctgcccgtgagcagcagggcc

SEQ ID NO: 8 - protein, 177 aa, synthetic construct; identical to SEQ ID NO: 4
  REGION 1..177: note = Murine IL9Ra ICD;;

SEQ ID NO: 9 - DNA, 531 bp, synthetic construct; identical to SEQ ID NO: 7
  misc_feature 1..531: note = Murine IL9Ra ICD;;
  source 1..531: mol_type = other DNA

SEQ ID NO: 10-48 - skipped (39 entries)

SEQ ID NO: 49 - protein, 262 aa, synthetic construct
  REGION 1..262: note = Anti-human mesothelin (anti-hMSLN) scFv;;
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 50 - DNA, 786 bp, synthetic construct
  misc_feature 1..786: note = Anti-human mesothelin (anti-hMSLN) scFv;;
  source 1..786: mol_type = other DNA
atggccctgcccgtgaccgccctgctgctgcccctggccctgctgctgcacgccgccaggccccaggtgcagctggtgcagagcggcgccgaggtggagaagcccggcgccagcgtgaaggtgagctgcaaggccagcggctacaccttcaccgactactacatgcactgggtgaggcaggcccccggccagggcctggagtggatgggctggatcaaccccaacagcggcggcaccaactacgcccagaagttccagggcagggtgaccatgaccagggacaccagcatcagcaccgcctacatggagctgagcaggctgaggagcgacgacaccgccgtgtactactgcgccagcggctgggacttcgactactggggccagggcaccctggtgaccgtgagcagcggcggcggcggcagcggcggcggcggcagcggcggcggcggcagcggcggcggcggcagcgacatcgtgatgacccagagccccagcagcctgagcgccagcgtgggcgacagggtgaccatcacctgcagggccagccagagcatcaggtactacctgagctggtaccagcagaagcccggcaaggcccccaagctgctgatctacaccgccagcatcctgcagaacggcgtgcccagcaggttcagcggcagcggcagcggcaccgacttcaccctgaccatcagcagcctgcagcccgaggacttcgccacctactactgcctgcagacctacaccacccccgacttcggccccggcaccaaggtggagatcaag

SEQ ID NO: 51 - protein, 45 aa, synthetic construct
  REGION 1..45: note = Human CD8 Hinge;;
TTTPAPRPPTPAPTIASQPLSLRPEACRPAAGGAVHTRGLDFACD

SEQ ID NO: 52 - DNA, 135 bp, synthetic construct
  misc_feature 1..135: note = Human CD8 Hinge;;
  source 1..135: mol_type = other DNA
accaccacccccgcccccaggccccccacccccgcccccaccatcgccagccagcccctgagcctgaggcccgaggcctgcaggcccgccgccggcggcgccgtgcacaccaggggcctggacttcgcctgcgac

SEQ ID NO: 53 - protein, 24 aa, synthetic construct
  REGION 1..24: note = Human CD8 TM;;
IYIWAPLAGTCGVLLLSLVITLYC

SEQ ID NO: 54 - DNA, 72 bp, synthetic construct
  misc_feature 1..72: note = Human CD8 TM;;
  source 1..72: mol_type = other DNA
atctacatctgggcccccctggccggcacctgcggcgtgctgctgctgagcctggtgatcaccctgtactgc

SEQ ID NO: 55 - protein, 69 aa, synthetic construct
  REGION 1..69: note = Human CD8 Hinge and TM;;
TTTPAPRPPTPAPTIASQPLSLRPEACRPAAGGAVHTRGLDFACDIYIWAPLAGTCGVLLLSLVITLYC

SEQ ID NO: 56 - DNA, 207 bp, synthetic construct
  misc_feature 1..207: note = Human CD8 Hinge and TM;;
  source 1..207: mol_type = other DNA
accaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgc

SEQ ID NO: 57 - protein, 27 aa, synthetic construct
  REGION 1..27: note = Human CD28 TM;;
FWVLVVVGGVLACYSLLVTVAFIIFWV

SEQ ID NO: 58 - DNA, 81 bp, synthetic construct
  misc_feature 1..81: note = Human CD28 TM;;
  source 1..81: mol_type = other DNA
ttctgggtgctggtggtggtgggcggcgtgctggcctgctacagcctgctggtgaccgtggccttcatcatcttctgggtg

SEQ ID NO: 59 - protein, 41 aa, synthetic construct
  REGION 1..41: note = Human CD28 costimulatory domain (hCD28 ICD);;
RSKRSRLLHSDYMNMTPRRPGPTRKHYQPYAPPRDFAAYRS

SEQ ID NO: 60 - DNA, 123 bp, synthetic construct
  misc_feature 1..123: note = Human CD28 costimulatory domain (hCD28 ICD);;
  source 1..123: mol_type = other DNA
aggagcaagaggagcaggctgctgcacagcgactacatgaacatgacccccaggaggcccggccccaccaggaagcactaccagccctacgccccccccagggacttcgccgcctacaggagc

SEQ ID NO: 61 - protein, 42 aa, synthetic construct
  REGION 1..42: note = Human 4-1BB costimulatory domain (h41BB ICD);;
KRGRKKLLYIFKQPFMRPVQTTQEEDGCSCRFPEEEEGGCEL

SEQ ID NO: 62 - DNA, 126 bp, synthetic construct
  misc_feature 1..126: note = Human 4-1BB costimulatory domain (h41BB ICD);;
  source 1..126: mol_type = other DNA
aagaggggcaggaagaagctgctgtacatcttcaagcagcccttcatgaggcccgtgcagaccacccaggaggaggacggctgcagctgcaggttccccgaggaggaggagggcggctgcgagctg

SEQ ID NO: 63 - protein, 112 aa, synthetic construct
  REGION 1..112: note = Human CD3z signaling domain (hCD3z ICD);;
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 336 bp, synthetic construct
  misc_feature 1..336: note = Human CD3z signaling domain (hCD3z ICD);;
  source 1..336: mol_type = other DNA
agggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 65 - protein, 277 aa, synthetic construct
  REGION 1..277: note = Anti-mouse mesothelin (anti-mMSLN) scFv;;
MASPLTRFLSLNLLLLGESIILGSGEATRAQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARFDYGDFYDAFDIWGQGTMVTVSSGGGGSGGGGSGGGGSEIVLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKRSG

SEQ ID NO: 66 - DNA, 831 bp, synthetic construct
  misc_feature 1..831: note = Anti-mouse mesothelin (anti-mMSLN) scFv;;
  source 1..831: mol_type = other DNA
atggctagtccgctcacgaggtttttgtctctgaaccttctcttattaggggaaagcataatcctgggcagcggcgaggctacgcgggcgcaggttcagctgcaagagtccggacccggtctggtgaagcccagccagactttgagcctgacctgtaccgtatctggtggctccataagttctggaggctactactggagctggataaggcagcacccagggaagggcctggagtggatcggctatatttactacagcgggagcacttattataatccctcattaaagagcagggtcaccatctcagtggacacatccaagaaccagttcagcttgaaactctcttccgtaacagctgctgacactgccgtttactattgtgccaggtttgactacggagatttttacgatgcctttgatatatggggccaaggcaccatggtgacagtctcctcaggtggaggaggcagtggggggggggggtctgggggtggtggctctgagatcgttctaacccagagcccgagcagcctatcggcgtcagtgggagatagagtgaccattacctgcagggcaagtcaaggcataagcagcgctctggcctggtaccaacaaaagcctggaaaggctcctaagctgctgatttatgatgcttcgagtctcgaaagtggtgtcccgtcaaggttttctggtagtggttcaggtacagacttcaccttgactatcagctcgctccaaccagaagatttcgcaacatattactgccagcagttcaacagctaccccattacatttggacaaggaacccggcttgaaattaaacgctcaggg

SEQ ID NO: 67 - protein, 51 aa, synthetic construct
  REGION 1..51: note = Mouse CD8 Hinge;;
LQKVNSTTTKPVLRTPSPVHPTGTSQPQRPEDCRPRGSVKGTGLDFACDIY

SEQ ID NO: 68 - DNA, 153 bp, synthetic construct
  misc_feature 1..153: note = Mouse CD8 Hinge;;
  source 1..153: mol_type = other DNA
cttcagaaggtgaacagcacaacaaccaagccagtcttgcgaacacccagtcctgttcaccctacgggtacgtctcaacctcagaggcctgaggactgtagaccccgtggctctgtgaaagggacagggctggactttgcttgtgacatctac

SEQ ID NO: 69 - protein, 21 aa, synthetic construct
  REGION 1..21: note = Mouse CD8 TM;;
IWAPLAGICVALLLSLIITLI

SEQ ID NO: 70 - DNA, 63 bp, synthetic construct
  misc_feature 1..63: note = Mouse CD8 TM;;
  source 1..63: mol_type = other DNA
atctgggcacccttagccggtatctgtgtggccttgctgctttccctcatcatcactctaatt

SEQ ID NO: 71 - protein, 72 aa, synthetic construct
  REGION 1..72: note = Mouse CD8 Hinge and TM;;
LQKVNSTTTKPVLRTPSPVHPTGTSQPQRPEDCRPRGSVKGTGLDFACDIYIWAPLAGICVALLLSLIITLI

SEQ ID NO: 72 - DNA, 216 bp, synthetic construct
  misc_feature 1..216: note = Mouse CD8 Hinge and TM;;
  source 1..216: mol_type = other DNA
cttcagaaggtgaacagcacaacaaccaagccagtcttgcgaacacccagtcctgttcaccctacgggtacgtctcaacctcagaggcctgaggactgtagaccccgtggctctgtgaaagggacagggctggactttgcttgtgacatctacatctgggcacccttagccggtatctgtgtggccttgctgctttccctcatcatcactctaatt

SEQ ID NO: 73 - protein, 27 aa, synthetic construct
  REGION 1..27: note = Mouse CD28 TM;;
FWALVVVAGVLFCYGLLVTVALCVIWT

SEQ ID NO: 74 - DNA, 81 bp, synthetic construct
  misc_feature 1..81: note = Mouse CD28 TM;;
  source 1..81: mol_type = other DNA
ttctgggccctggtggtggtggccggcgtgctgttctgctacggcctgctggtgaccgtggccctgtgcgtgatctggacc

SEQ ID NO: 75 - protein, 41 aa, synthetic construct
  REGION 1..41: note = Mouse CD28 costimulatory domain (mCD28 ICD);;
NSRRNRLLQSDYMNMTPRRPGLTRKPYQPYAPARDFAAYRP

SEQ ID NO: 76 - DNA, 123 bp, synthetic construct
  misc_feature 1..123: note = Mouse CD28 costimulatory domain (mCD28 ICD);;
  source 1..123: mol_type = other DNA
aacagcaggaggaacaggctgctgcagagcgactacatgaacatgacccccaggaggcccggcctgaccaggaagccctaccagccctacgcccccgccagggacttcgccgcctacaggccc

SEQ ID NO: 77 - protein, 45 aa, synthetic construct
  REGION 1..45: note = Mouse 4-1BB costimulatory domain (m41BB ICD);;
KWIRKKFPHIFKQPFKKTTGAAQEEDACSCRCPQEEEGGGGGYEL

SEQ ID NO: 78 - DNA, 135 bp, synthetic construct
  misc_feature 1..135: note = Mouse 4-1BB costimulatory domain (m41BB ICD);;
  source 1..135: mol_type = other DNA
aagtggattcgaaaaaagttcccccacatctttaagcagccgttcaagaaaaccactggagcagcccaggaggaggatgcttgcagctgccgctgtccccaggaggaagaaggcggcgggggcggatatgagctc

SEQ ID NO: 79 - protein, 111 aa, synthetic construct
  REGION 1..111: note = Mouse CD3z signaling domain (mCD3z ICD);;
KFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 80 - DNA, 333 bp, synthetic construct
  misc_feature 1..333: note = Mouse CD3z signaling domain (mCD3z ICD);;
  source 1..333: mol_type = other DNA
aagttttcacgctctgcagagacagctgccaacctgcaggaccccaatcagctgtacaatgaactgaatctcgggcggagagaagaatatgatgtgttggagaagaagcgtgcgagagacccagagatgggcggcaaacagcagagaagacgaaacccacaggaaggagtgtacaacgccctgcagaaagacaagatggcagaggcctactcagagattggaaccaaaggagagaggcgccgtggaaaaggacatgatgggctttaccagggtttaagtacggccactaaagatacttatgacgcgctgcacatgcagacactggcacctcga

SEQ ID NO: 81 - protein, 717 aa, synthetic construct
  REGION 1..717: note = Human anti-hMSLN scFv hCD8 hinge hCD28 TM and ICD hIL9Ra ICD hCD3z;;
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 2151 bp, synthetic construct
  misc_feature 1..2151: note = Human anti-hMSLN scFv hCD8 hinge hCD28 TM and ICD hIL9Ra ICD hCD3z;;
  source 1..2151: mol_type = other DNA
atggccctgcccgtgaccgccctgctgctgcccctggccctgctgctgcacgccgccaggccccaggtgcagctggtgcagagcggcgccgaggtggagaagcccggcgccagcgtgaaggtgagctgcaaggccagcggctacaccttcaccgactactacatgcactgggtgaggcaggcccccggccagggcctggagtggatgggctggatcaaccccaacagcggcggcaccaactacgcccagaagttccagggcagggtgaccatgaccagggacaccagcatcagcaccgcctacatggagctgagcaggctgaggagcgacgacaccgccgtgtactactgcgccagcggctgggacttcgactactggggccagggcaccctggtgaccgtgagcagcggcggcggcggcagcggcggcggcggcagcggcggcggcggcagcggcggcggcggcagcgacatcgtgatgacccagagccccagcagcctgagcgccagcgtgggcgacagggtgaccatcacctgcagggccagccagagcatcaggtactacctgagctggtaccagcagaagcccggcaaggcccccaagctgctgatctacaccgccagcatcctgcagaacggcgtgcccagcaggttcagcggcagcggcagcggcaccgacttcaccctgaccatcagcagcctgcagcccgaggacttcgccacctactactgcctgcagacctacaccacccccgacttcggccccggcaccaaggtggagatcaagaccaccacccccgcccccaggccccccacccccgcccccaccatcgccagccagcccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcaagctgagccccagggtgaagaggatcttctaccagaacgtgcccagccccgccatgttcttccagcccctgtacagcgtgcacaacggcaacttccagacctggatgggcgcccacggcgccggcgtgctgctgagccaggactgcgccggcaccccccagggcgccctggagccctgcgtgcaggaggccaccgccctgctgacctgcggccccgccaggccctggaagagcgtggccctggaggaggagcaggagggccccggcaccaggctgcccggcaacctgagcagcgaggacgtgctgcccgccggctgcaccgagtggagggtgcagaccctggcctacctgccccaggaggactgggcccccaccagcctgaccaggcccgccccccccgacagcgagggcagcaggagcagcagcagcagcagcagcagcaacaacaacaactactgcgccctgggctgctacggcggctggcacctgagcgccctgcccggcaacacccagagcagcggccccatccccgccctggcctgcggcctgagctgcgaccaccagggcctggagacccagcagggcgtggcctgggtgctggccggccactgccagaggcccggcctgcacgaggacctgcagggcatgctgctgcccagcgtgctgagcaaggccaggagctggaccttcagggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 83 - protein, 715 aa, synthetic construct
  REGION 1..715: note = Human anti-hMSLN scFv hCD8 hinge and TM h41BB ICD hIL9Ra ICD hCD3z ;;
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - DNA, 2145 bp, synthetic construct
  misc_feature 1..2145: note = Human anti-hMSLN scFv hCD8 hinge and TM h41BB ICD hIL9Ra ICD hCD3z ;;
  source 1..2145: mol_type = other DNA
atggccctgcccgtgaccgccctgctgctgcccctggccctgctgctgcacgccgccaggccccaggtgcagctggtgcagagcggcgccgaggtggagaagcccggcgccagcgtgaaggtgagctgcaaggccagcggctacaccttcaccgactactacatgcactgggtgaggcaggcccccggccagggcctggagtggatgggctggatcaaccccaacagcggcggcaccaactacgcccagaagttccagggcagggtgaccatgaccagggacaccagcatcagcaccgcctacatggagctgagcaggctgaggagcgacgacaccgccgtgtactactgcgccagcggctgggacttcgactactggggccagggcaccctggtgaccgtgagcagcggcggcggcggcagcggcggcggcggcagcggcggcggcggcagcggcggcggcggcagcgacatcgtgatgacccagagccccagcagcctgagcgccagcgtgggcgacagggtgaccatcacctgcagggccagccagagcatcaggtactacctgagctggtaccagcagaagcccggcaaggcccccaagctgctgatctacaccgccagcatcctgcagaacggcgtgcccagcaggttcagcggcagcggcagcggcaccgacttcaccctgaccatcagcagcctgcagcccgaggacttcgccacctactactgcctgcagacctacaccacccccgacttcggccccggcaccaaggtggagatcaagaccaccacccccgcccccaggccccccacccccgcccccaccatcgccagccagcccctgagcctgaggcccgaggcctgcaggcccgccgccggcggcgccgtgcacaccaggggcctggacttcgcctgcgacatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgaagctgagccccagggtgaagaggatcttctaccagaacgtgcccagccccgccatgttcttccagcccctgtacagcgtgcacaacggcaacttccagacctggatgggcgcccacggcgccggcgtgctgctgagccaggactgcgccggcaccccccagggcgccctggagccctgcgtgcaggaggccaccgccctgctgacctgcggccccgccaggccctggaagagcgtggccctggaggaggagcaggagggccccggcaccaggctgcccggcaacctgagcagcgaggacgtgctgcccgccggctgcaccgagtggagggtgcagaccctggcctacctgccccaggaggactgggcccccaccagcctgaccaggcccgccccccccgacagcgagggcagcaggagcagcagcagcagcagcagcagcaacaacaacaactactgcgccctgggctgctacggcggctggcacctgagcgccctgcccggcaacacccagagcagcggccccatccccgccctggcctgcggcctgagctgcgaccaccagggcctggagacccagcagggcgtggcctgggtgctggccggccactgccagaggcccggcctgcacgaggacctgcagggcatgctgctgcccagcgtgctgagcaaggccaggagctggaccttcagggtgaagttcagcaggagcgccgacgcccccgcctacaagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 85 - protein, 684 aa, synthetic construct
  REGION 1..684: note = Mouse anti-mMSLN scFv mCD8 hinge mCD28 TM and ICD mIL9Ra ICD mCD3z;;
MASPLTRFLSLNLLLLGESIILGSGEATRAQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARFDYGDFYDAFDIWGQGTMVTVSSGGGGSGGGGSGGGGSEIVLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKRSGLQKVNSTTTKPVLRTPSPVHPTGTSQPQRPEDCRPRGSVKGTGLDFACDIYFWALVVVAGVLFCYGLLVTVALCVIWTNSRRNRLLQSDYMNMTPRRPGLTRKPYQPYAPARDFAAYRPKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDSGSSDYCMLDCCEECHLSAFPGHTESPELTLAQPVALPVSSRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 86 - DNA, 2052 bp, synthetic construct
  misc_feature 1..2052: note = Mouse anti-mMSLN scFv mCD8 hinge mCD28 TM and ICD mIL9Ra ICD mCD3z;;
  source 1..2052: mol_type = other DNA
atggctagtccgctcacgaggtttttgtctctgaaccttctcttattaggggaaagcataatcctgggcagcggcgaggctacgcgggcgcaggttcagctgcaagagtccggacccggtctggtgaagcccagccagactttgagcctgacctgtaccgtatctggtggctccataagttctggaggctactactggagctggataaggcagcacccagggaagggcctggagtggatcggctatatttactacagcgggagcacttattataatccctcattaaagagcagggtcaccatctcagtggacacatccaagaaccagttcagcttgaaactctcttccgtaacagctgctgacactgccgtttactattgtgccaggtttgactacggagatttttacgatgcctttgatatatggggccaaggcaccatggtgacagtctcctcaggtggaggaggcagtggggggggggggtctgggggtggtggctctgagatcgttctaacccagagcccgagcagcctatcggcgtcagtgggagatagagtgaccattacctgcagggcaagtcaaggcataagcagcgctctggcctggtaccaacaaaagcctggaaaggctcctaagctgctgatttatgatgcttcgagtctcgaaagtggtgtcccgtcaaggttttctggtagtggttcaggtacagacttcaccttgactatcagctcgctccaaccagaagatttcgcaacatattactgccagcagttcaacagctaccccattacatttggacaaggaacccggcttgaaattaaacgctcagggcttcagaaggtgaacagcacaacaaccaagccagtcttgcgaacacccagtcctgttcaccctacgggtacgtctcaacctcagaggcctgaggactgtagaccccgtggctctgtgaaagggacagggctggactttgcttgtgacatctacttctgggccctggtggtggtggccggcgtgctgttctgctacggcctgctggtgaccgtggccctgtgcgtgatctggaccaacagcaggaggaacaggctgctgcagagcgactacatgaacatgacccccaggaggcccggcctgaccaggaagccctaccagccctacgcccccgccagggacttcgccgcctacaggcccaaactatctccacggctcaaacggatcttctaccagaacatcccttctcctgaagcattcttccatcccctgtattcagtttatcatggagacttccagtcctggactggggcccgcagagctgggccacaagctcgacagaatggcgtgtccaccagctctgcagggtccgagtcttccatttgggaggcagtggcaactctgacttactccccagcatgccctgtgcagtttgcctgtctgaaatgggaagccactgccccgggcttcccaggattgccgggcagtgagcatgtcctgcctgcaggctgcctcgaactcgagggccagccatctgcctacctgccccaagaagactgggccccactcggctcagctagacctccccccccagatagtgactccgggtcgtctgactattgcatgctggattgctgtgaagagtgccacctgagtgccttccctggccacacagagagtcccgagctgaccttggctcagccagtagccctccctgtcagctcccgggccaagttttcacgctctgcagagacagctgccaacctgcaggaccccaatcagctgtacaatgaactgaatctcgggcggagagaagaatatgatgtgttggagaagaagcgtgcgagagacccagagatgggcggcaaacagcagagaagacgaaacccacaggaaggagtgtacaacgccctgcagaaagacaagatggcagaggcctactcagagattggaaccaaaggagagaggcgccgtggaaaaggacatgatgggctttaccagggtttaagtacggccactaaagatacttatgacgcgctgcacatgcagacactggcacctcga

SEQ ID NO: 87 - protein, 682 aa, synthetic construct
  REGION 1..682: note = Mouse anti-mMSLN scFv mCD8 hinge and TM m41BB ICD mIL9Ra ICD mCD3z ;;
MASPLTRFLSLNLLLLGESIILGSGEATRAQVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARFDYGDFYDAFDIWGQGTMVTVSSGGGGSGGGGSGGGGSEIVLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKRSGLQKVNSTTTKPVLRTPSPVHPTGTSQPQRPEDCRPRGSVKGTGLDFACDIYIWAPLAGICVALLLSLIITLIKWIRKKFPHIFKQPFKKTTGAAQEEDACSCRCPQEEEGGGGGYELKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDSGSSDYCMLDCCEECHLSAFPGHTESPELTLAQPVALPVSSRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 88 - DNA, 2046 bp, synthetic construct
  misc_feature 1..2046: note = Mouse anti-mMSLN scFv mCD8 hinge and TM m41BB ICD mIL9Ra ICD mCD3z ;;
  source 1..2046: mol_type = other DNA
atggctagtccgctcacgaggtttttgtctctgaaccttctcttattaggggaaagcataatcctgggcagcggcgaggctacgcgggcgcaggttcagctgcaagagtccggacccggtctggtgaagcccagccagactttgagcctgacctgtaccgtatctggtggctccataagttctggaggctactactggagctggataaggcagcacccagggaagggcctggagtggatcggctatatttactacagcgggagcacttattataatccctcattaaagagcagggtcaccatctcagtggacacatccaagaaccagttcagcttgaaactctcttccgtaacagctgctgacactgccgtttactattgtgccaggtttgactacggagatttttacgatgcctttgatatatggggccaaggcaccatggtgacagtctcctcaggtggaggaggcagtggggggggggggtctgggggtggtggctctgagatcgttctaacccagagcccgagcagcctatcggcgtcagtgggagatagagtgaccattacctgcagggcaagtcaaggcataagcagcgctctggcctggtaccaacaaaagcctggaaaggctcctaagctgctgatttatgatgcttcgagtctcgaaagtggtgtcccgtcaaggttttctggtagtggttcaggtacagacttcaccttgactatcagctcgctccaaccagaagatttcgcaacatattactgccagcagttcaacagctaccccattacatttggacaaggaacccggcttgaaattaaacgctcagggcttcagaaggtgaacagcacaacaaccaagccagtcttgcgaacacccagtcctgttcaccctacgggtacgtctcaacctcagaggcctgaggactgtagaccccgtggctctgtgaaagggacagggctggactttgcttgtgacatctacatctgggcacccttagccggtatctgtgtggccttgctgctttccctcatcatcactctaattaagtggattcgaaaaaagttcccccacatctttaagcagccgttcaagaaaaccactggagcagcccaggaggaggatgcttgcagctgccgctgtccccaggaggaagaaggcggcgggggcggatatgagctcaaactatctccacggctcaaacggatcttctaccagaacatcccttctcctgaagcattcttccatcccctgtattcagtttatcatggagacttccagtcctggactggggcccgcagagctgggccacaagctcgacagaatggcgtgtccaccagctctgcagggtccgagtcttccatttgggaggcagtggcaactctgacttactccccagcatgccctgtgcagtttgcctgtctgaaatgggaagccactgccccgggcttcccaggattgccgggcagtgagcatgtcctgcctgcaggctgcctcgaactcgagggccagccatctgcctacctgccccaagaagactgggccccactcggctcagctagacctccccccccagatagtgactccgggtcgtctgactattgcatgctggattgctgtgaagagtgccacctgagtgccttccctggccacacagagagtcccgagctgaccttggctcagccagtagccctccctgtcagctcccgggccaagttttcacgctctgcagagacagctgccaacctgcaggaccccaatcagctgtacaatgaactgaatctcgggcggagagaagaatatgatgtgttggagaagaagcgtgcgagagacccagagatgggcggcaaacagcagagaagacgaaacccacaggaaggagtgtacaacgccctgcagaaagacaagatggcagaggcctactcagagattggaaccaaaggagagaggcgccgtggaaaaggacatgatgggctttaccagggtttaagtacggccactaaagatacttatgacgcgctgcacatgcagacactggcacctcga

SEQ ID NO: 89-97 - skipped (9 entries)

SEQ ID NO: 98 - protein, 239 aa, synthetic construct
  REGION 1..239: note = Anti-EGFR (806) scFv;;
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 99 - DNA, 717 bp, synthetic construct
  misc_feature 1..717: note = Anti-EGFR (806) scFv;;
  source 1..717: mol_type = other DNA
gatattctgatgactcaatctccgtcttctatgagcgtgagcttgggtgacaccgtcagcatcacctgtcattccagccaggatataaactcaaatatcggctggctccagcaacgcccaggcaagtcattcaaggggcttatttatcatggcaccaatcttgacgatgaagtcccatcacgcttcagcggatcaggctcaggtgcggactattccttgactataagttccctcgaatctgaggatttcgccgactattattgcgtacaatacgcccagtttccctggaccttcggaggcggcaccaaattggagataaaaaggggtggaggaggatcaggcgggggtggaagcggcggaggaggcagcgacgtacaactgcaagaatccgggccgagtttggtcaagccctctcaatctctttctctcacttgcacggtcaccggatactccataaccagcgattttgcgtggaattggattcgacaatttccagggaataaattggaatggatgggatatatcagttattctggtaataccagatacaacccgtcattgaaaagtcgcatctctataacacgagacacttcaaagaatcagttcttccttcagctcaattctgtaaccatcgaagatactgctacttattactgtgtaacggcgggtcgaggattcccctactggggccagggtacactggttactgtttccgcc

SEQ ID NO: 100 - protein, 244 aa, synthetic construct
  REGION 1..244: note = Anti-IL13Ra2 (hu08) scFv;;
DIQMTQSPSSLSASVGDRVTITCKASQDVGTAVAWYQQIPGKAPKLLIYSASYRSTGVPDRFSGSGSGTDFSFIISSLQPEDFATYYCQHHYSAPWTFGGGTKVEIKGGGGSGGGGSGGGGSEVQLVESGGGLVQPGGSLRLSCAASGFTFSRNGMSWVRQTPDKRLEWVATVSSGGSYIYYADSVKGRFTISRDNAKNSLYLQMSSLRAEDTAVYYCARQGTTALATRFFDVWGQGTLVTVSS

SEQ ID NO: 101 - DNA, 731 bp, synthetic construct
  misc_feature 1..731: note = Anti-IL13Ra2 (hu08) scFv;;
  source 1..731: mol_type = other DNA
gacatccaaatgactcagagcccctctagcctcagtgcaagcgtcggagaccgggtgaccatcacctgtaaagcgtcccaggatgttggaacggcagtagcttggtatcaacaaatcccagggaaggctccaaagctccttatatactctgctagttacaggtccaccggggtgcccgaccgattctctggctccgggagcggcactgacttttcattcatcattagtagtcttcaacctgaggactttgccacctattattgccagcaccactactctgcgccgtggactttcggaggaggcacgaaggttgaaattaaaggtggaggtgggtctggcggaggtggaagtggtggaggcgggtccgaggttcagttggtagagtcaggcggtggtctggtgcagccaggtgggtccctgcgcctcagctgtgcagcttccggctttactttctcaaggaatggtatgtcctgggtacggcaaacgccggacaaacgccttgagtgggtagctaccgtatcctctgggggctcttacatatactatgcagactctgtgaaaggaagatttacaatttcacgcgacaatgcaaaaaatagtttgtacctccaaatgtctagtcttagggccgaggatactgccgtctactactgtgcacgccagggaacgacggctcttgctacccgatttttcgacgtttggggccaaggaacgttggtgacagttagcag

SEQ ID NO: 102 - protein, 11 aa, synthetic construct
  REGION 1..11: note = Anti-hMSLN scFv M5 LCDR1;;
RASQSIRYYLS

SEQ ID NO: 103 - protein, 7 aa, synthetic construct
  REGION 1..7: note = Anti-hMSLN scFv M5 LCDR2;;
TASILQN

SEQ ID NO: 104 - protein, 8 aa, synthetic construct
  REGION 1..8: note = Anti-hMSLN scFv M5 LCDR3;;
LQTYTTPD

SEQ ID NO: 105 - protein, 10 aa, synthetic construct
  REGION 1..10: note = Anti-hMSLN scFv M5 HCDR1;;
GYTFTDYYMH

SEQ ID NO: 106 - protein, 17 aa, synthetic construct
  REGION 1..17: note = Anti-hMSLN scFv M5 HCDR2;;
WINPNSGGTNYAQKFQG

SEQ ID NO: 107 - protein, 6 aa, synthetic construct
  REGION 1..6: note = Anti-hMSLN scFv M5 HCDR3;;
GWDFDY

SEQ ID NO: 108 - protein, 270 aa, synthetic construct
  REGION 1..270: note = Anti-GD2 scFv;;
MALPVTALLLPLALLLHAARPGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSGGGGSEVQLLQSGPELEKPSASVMISCKASGSSFTGYNMNWVRQNIGKSLEWIGAIDPYYGGTSYNQKFKGRATLTVDKSSSTAYMHLKSLTSEDSVYYCVSGMEYWGQGTSVTVSSSG

SEQ ID NO: 109 - DNA, 810 bp, synthetic construct
  misc_feature 1..810: note = Anti-GD2 scFv;;
  source 1..810: mol_type = other DNA
atggccttaccagtgaccgccttgctcctgccgctggccttgctgctccacgccgccaggccgggatccgatgttgtgatgacccaaactccactctccctgcctgtcagtcttggagatcaagcctccatctcttgcagatctagtcagagtcttgtacaccgtaacggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacacgttcctccgctcacgttcggtgctgggaccaagctggagctgaaaggaggtggcgggtcagggggtggcggaagcggaggcggcggttcaggcggaggaggctcggaggtgcagcttctgcagtctggacctgagctggagaagccttccgcttcagtgatgatatcctgcaaggcttctggttcctccttcactggctacaacatgaactgggtgaggcagaatattggaaagagccttgaatggattggagctattgatccttactacggtggaactagctacaaccagaagttcaagggcagggccacattgactgtagacaaatcgtccagcacagcctacatgcacctcaagagcctgacatctgaggactctgtctattactgtgtaagcggaatggagtactggggtcaaggaacctcagtcaccgtctcctcatccgga

SEQ ID NO: 110 - protein, 266 aa, synthetic construct
  REGION 1..266: note = Anti-HER2 scFv (high affinity);;
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 111 - DNA, 798 bp, synthetic construct
  misc_feature 1..798: note = Anti-HER2 scFv (high affinity);;
  source 1..798: mol_type = other DNA
atggattttcaggtgcagattttcagcttcctgctaatcagtgcctcagtcataatgtccagaggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctttattctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcg

SEQ ID NO: 112 - protein, 266 aa, synthetic construct
  REGION 1..266: note = Anti-HER2 scFv (low affinity);;
MDFQVQIFSFLLISASVIMSRGDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFVAMDVWGQGTLVTVSS

SEQ ID NO: 113 - DNA, 798 bp, synthetic construct
  misc_feature 1..798: note = Anti-HER2 scFv (low affinity);;
  source 1..798: mol_type = other DNA
atggattttcaggtgcagattttcagcttcctgctaatcagtgcctcagtcataatgtccagaggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttcgttgctatggacgtgtggggtcaaggaaccctggtcaccgtctcctcg

SEQ ID NO: 114 - protein, 244 aa, synthetic construct
  REGION 1..244: note = Anti-TnMuc1 scFv;;
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 115 - DNA, 732 bp, synthetic construct
  misc_feature 1..732: note = Anti-TnMuc1 scFv;;
  source 1..732: mol_type = other DNA
caggtgcagctgcagcagtctgatgccgagctcgtgaagcctggcagcagcgtgaagatcagctgcaaggccagcggctacaccttcaccgaccacgccatccactgggtcaagcagaagcctgagcagggcctggagtggatcggccacttcagccccggcaacaccgacatcaagtacaacgacaagttcaagggcaaggccaccctgaccgtggacagaagcagcagcaccgcctacatgcagctgaacagcctgaccagcgaggacagcgccgtgtacttctgcaagaccagcaccttctttttcgactactggggccagggcacaaccctgacagtgtctagcggaggcggaggatctggcggcggaggaagtggcggagggggatctgaactcgtgatgacccagagccccagctctctgacagtgacagccggcgagaaagtgaccatgatctgcaagtcctcccagagcctgctgaactccggcgaccagaagaactacctgacctggtatcagcagaaacccggccagccccccaagctgctgatcttttgggccagcacccgggaaagcggcgtgcccgatagattcacaggcagcggctccggcaccgactttaccctgaccatcagctccgtgcaggccgaggacctggccgtgtattactgccagaacgactacagctaccccctgaccttcggagccggcaccaagctggaactgaag

SEQ ID NO: 116 - protein, 274 aa, synthetic construct
  REGION 1..274: note = Anti-CD70 scFv;;
MALPVTALLLPLALLLHAARPQAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLGGGGGSGGGGSGGGGSGGGGSEVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSS

SEQ ID NO: 117 - DNA, 822 bp, synthetic construct
  misc_feature 1..822: note = Anti-CD70 scFv;;
  source 1..822: mol_type = other DNA
atggccttaccagtgaccgccttgctcctgccgctggccttgctgctccacgccgccaggccgcaggccgttgtcacacaagagccctctctgaccgtgtcacccggggggaccgtgaccctgacatgtggtttgaaatctggaagtgtaacatccgacaactttcctacatggtaccagcagacgcctggtcaagcgccgcgattgctgatttataataccaatacacgccacagcggagtacctgacagattcagtggcagcatccttggaaacaaagcggcactgaccataacaggtgcccaagcagatgatgaagcagagtacttctgtgccctctttattagtaatccctcagttgaatttgggggtggtacacaacttacagttctcggtggtggcggaggatcaggggggggaggaagtggtggtggcggcagtggcggaggtgggagtgaggttcagctcgtagaatcaggaggaggtttggtacaaccgggcggctctctgagactttcatgcgctgcgagcgggtttactttctctgtctattatatgaattgggtgagacaggcgccgggaaaggggctggaatgggtgagtgatattaacaatgaaggaggtaccacctactacgcggacagtgtaaagggcagatttaccataagccgggataacagtaagaacagtctttacttgcaaatgaattcactgcgagcggaggataccgcggtatactactgtgctagggacgcgggttacagtaaccatgtgccaattttcgattcttggggacagggaaccctcgtcaccgtgtccagc

SEQ ID NO: 118 - DNA, 1113 bp, synthetic construct
  misc_feature 1..1113: note = Anti-CD70 tr27 CAR;;
  source 1..1113: mol_type = other DNA
atggctcggccccatccctggtggttgtgtgtgctgggaacacttgtcggcctgagtgctacccctgcccctaaatcatgcccggaacggcactattgggcgcagggtaaactgtgctgccaaatgtgtgaaccaggtacttttctggtcaaagattgcgatcaacacaggaaggcagctcaatgcgatccttgtatccctggggtgagcttcagccccgaccatcatactagaccacattgtgaaagttgccgacactgtaatagcggactcttggtccgcaattgcaccattaccgctaatgccgaatgcgcctgccgcaatggatggcagtgccgggacaaggagtgcacagagtgcgaccctctcccaaatccaagtctcacggctcggtccagtcaggcgcttagcccgcacccacaacctactcacctgccctacgtctctgaaatgttggaagcgagaacagcaggtcacatgcaaacacttgcggactttcggcagctgcctgcgcgcacactttcaacccattggccaccacaacggagtctgtgtagttccgacttcataagaatcctcgttatcttctctgggatgttcttggtattcacgttggccggcgccctgtttctccggttcagtgtagtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 119 - protein, 370 aa, synthetic construct
  REGION 1..370: note = Anti-CD70 tr27 CAR;;
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 264 aa, synthetic construct
  REGION 1..264: note = Anti-PMSA scFv;;
MALPVTALLLPLALLLHAARPGSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKGGGGSGGGGSSGGGSEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSASSG

SEQ ID NO: 121 - DNA, 792 bp, synthetic construct
  misc_feature 1..792: note = Anti-PMSA scFv;;
  source 1..792: mol_type = other DNA
atggccctgcctgtgacagccctgctgctgcctctggctctgctgctgcacgccgccagacctggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaacgttcagtctgaagacttggcagattatttctgtcagcaatataacagctatcctctcacgttcggtgctgggaccatgctggacctgaaaggaggcggaggatctggcggcggaggaagttctggcggaggcagcgaggtgcagctgcagcagagcggacccgagctcgtgaagcctggaacaagcgtgcggatcagctgcaagaccagcggctacaccttcaccgagtacaccatccactgggtcaagcagtcccacggcaagagcctggagtggatcggcaatatcaaccccaacaacggcggcaccacctacaaccagaagttcgaggacaaggccaccctgaccgtggacaagagcagcagcaccgcctacatggaactgcggagcctgaccagcgaggacagcgccgtgtactattgtgccgccggttggaacttcgactactggggccagggcacaaccctgacagtgtctagcgctagctccgga

SEQ ID NO: 122 - protein, 267 aa, synthetic construct
  REGION 1..267: note = Anti-EGFRvIII scFv;;
MALPVTALLLPLALLLHAARPEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 123 - DNA, 801 bp, synthetic construct
  misc_feature 1..801: note = Anti-EGFRvIII scFv;;
  source 1..801: mol_type = other DNA
atggccctccctgtcaccgccctgctgcttccgctggctcttctgctccacgccgctcggcccgagattcagctcgtgcaatcgggagcggaagtcaagaagccaggagagtccttgcggatctcatgcaagggtagcggctttaacatcgaggattactacatccactgggtgaggcagatgccggggaagggactcgaatggatgggacggatcgacccagaaaacgacgaaactaagtacggtccgatcttccaaggccatgtgactattagcgccgatacttcaatcaataccgtgtatctgcaatggtcctcattgaaagcctcagataccgcgatgtactactgtgctttcagaggaggggtctactggggacagggaactaccgtgactgtctcgtccggcggaggcgggtcaggaggtggcggcagcggaggaggagggtccggcggaggtgggtccgacgtcgtgatgacccagagccctgacagcctggcagtgagcctgggcgaaagagctaccattaactgcaaatcgtcgcagagcctgctggactcggacggaaaaacgtacctcaattggctgcagcaaaagcctggccagccaccgaagcgccttatctcactggtgtcgaagctggattcgggagtgcccgatcgcttctccggctcgggatcgggtactgacttcaccctcactatctcctcgcttcaagcagaggacgtggccgtctactactgctggcagggaacccactttccgggaaccttcggcggagggacgaaagtggagatcaag

SEQ ID NO: 124 - DNA, 531 bp, synthetic construct
  misc_feature 1..531: note = Murine IL9Ra ICD;;
  source 1..531: mol_type = other DNA
aaactatctccacggctcaaacggatcttctaccagaacatcccttctcctgaagcattcttccatcccctgtattcagtttatcatggagacttccagtcctggactggggcccgcagagctgggccacaagctcgacagaatggcgtgtccaccagctctgcagggtccgagtcttccatttgggaggcagtggcaactctgacttactccccagcatgccctgtgcagtttgcctgtctgaaatgggaagccactgccccgggcttcccaggattgccgggcagtgagcatgtcctgcctgcaggctgcctcgaactcgagggccagccatctgcctacctgccccaagaagactgggccccactcggctcagctagacctccccccccagatagtgactccgggtcgtctgactattgcatgctggattgctgtgaagagtgccacctgagtgccttccctggccacacagagagtcccgagctgaccttggctcagccagtagccctccctgtcagctcccgggcc

(19) United States
(12) Patent Application Publication
(10) Pub. No.: US 2023/0085834 A1
(43) Pub. Date: Mar. 23, 2023

(54) Chimeric Antigen Receptors Comprising Interleukin-9 Receptor Signaling Domain

(71) Applicants: THE TRUSTEES OF THE UNIVERSITY OF PENNSYLVANIA, Philadelphia, PA (US); THE BOARD OF TRUSTEES OF THE LELAND STANFORD JUNIOR UNIVERSITY, Stanford, CA (US)

(72) Inventors: Mikko SIURALA, Philadelphia, PA (US); Carl H. JUNE, Merion Station, PA (US); Kenan Christopher GARCIA, Stanford, CA (US)

(21) Appl. No.: 17/933,049

(22) Filed: Sep. 16, 2022

Related U.S. Application Data
(60) Provisional application No. 63/245,386, filed on Sep. 17, 2021.
(60) Provisional application No. 63/245,400, filed on Sep. 17, 2021.

(51) Int. Cl. C07K 14/715; C12N 15/62; C12N 15/86; C12N 5/0783; C07K 14/705; C07K 14/725; C07K 16/18; A61P 35/00; A61K 35/17

(52) U.S. Cl. CPC C07K 14/7155; C12N 15/625; C12N 15/86; C12N 5/0636; C07K 14/70521; C07K 14/70578; C07K 14/7051; C07K 14/70517; C07K 16/18; A61P 35/00; A61K 35/17; C12N 2740/10043; A61K 2039/505

(57) ABSTRACT

The present disclosure provides a CAR comprising a tumor antigen binding domain, a transmembrane domain, and an intracellular domain comprising an intracellular signaling domain of an interleukin-9 receptor alpha (IL9Ra), and modified cell(s), i.e., immune cell(s) or precursor cell(s) thereof, engineered to express the CAR. Also provided are methods and uses of the modified cells, e.g., for treating at least one sign and/or symptom of cancer. Related nucleic acids, vectors, and pharmaceutical compositions are also provided.

### CROSS-REFERENCE TO RELATED APPLICATIONS

This application claims priority under 35 U.S.C. § 119(e) to U.S. Provisional Patent Application No. 63/245,400, filed Sep. 17, 2021, and to U.S. Provisional Patent Application No. 63/245,386, filed Sep. 17, 2021, which are incorporated herein by reference in their entireties.

### INCORPORATION-BY-REFERENCE OF SEQUENCE LISTING

The present application contains a Sequence Listing which has been submitted in XML format via Patent Center and is hereby incorporated by reference in its entirety. Said XML file, created on Sep. 16, 2022, is named 046483-7347US1(02942) Sequence Listing.xml and is 134 kilobytes in size.

### BACKGROUND OF THE INVENTION

Current immunotherapy advances have been revolutionary for the treatment of hematologic malignancies as evident by the FDA approvals of CD19-targeting chimeric antigen receptor T cells (CAR-T cells) for the treatment of acute lymphoblastic leukemia and diffuse-large B-cell lymphoma. However, the greatest unmet burden for cancer treatment is solid tumors. CAR-T cells have lacked efficacy in the fight against solid tumors due to a number of challenges, including the lack of tumor-specific antigens, overcoming obstacles of therapeutic resistance, tumor heterogeneity, poor expansion and persistence, and extrinsic dysfunction and physical barriers to T cell infiltration caused by the dense, immunosuppressive tumor microenvironment (TME). One major limitation is the poor in vivo expansion and persistence of adoptively transferred T cells, necessitating lymphodepleting conditioning chemotherapy—a toxic regimen that limits patient eligibility. Even those T cells that do expand and persist become terminally differentiated and dysfunctional. T cells with a stem-like phenotype can overcome these limitations and exhibit superior antitumor activity in mouse models and humans, but therapeutic manipulations to select or expand stem-like T cells are limited to the cell manufacturing phase and cannot be made in vivo. There is a need in the art for novel cell-based therapies that overcome these obstacles and challenges. The present invention addresses this need.

### SUMMARY OF THE INVENTION

In some aspects, the invention provides a chimeric antigen receptor (CAR) comprising a tumor antigen binding domain, a transmembrane domain, and an intracellular domain comprising an intracellular signaling domain of an interleukin-9 receptor alpha (IL9Ra).

In some embodiments, the tumor antigen is selected from alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

In some embodiments, the tumor antigen is selected from mesothelin, GD2, HER2, TnMuc1, CD70, PMSA, and EGFRvIII.

In some embodiments, the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

In some embodiments, the tumor antigen binding domain is a single-chain variable fragment (scFv).

In some embodiments, the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

In some embodiments, the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular domain of the CAR further comprises a costimulatory domain of a CD28, a costimulatory domain of a 4-1BB, an intracellular signaling domain of a CD3 zeta, or any combination thereof.

In some embodiments, the CAR further comprises a hinge domain.

In some embodiments, the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

In some embodiments, the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

In some embodiments, the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

In another aspect, there is provided an isolated nucleic acid comprising a nucleotide sequence encoding a chimeric antigen receptor (CAR), wherein the CAR comprises a tumor antigen binding domain, a transmembrane domain, and an intracellular domain comprising an intracellular signaling domain of an interleukin-9 receptor alpha (IL9Ra).

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

In some embodiments, the tumor antigen is selected from mesothelin, GD2, HER2, TnMuc1, CD70, PMSA, and EGFRvIII.

In some embodiments, the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

In some embodiments, the tumor antigen binding domain is a single-chain variable fragment (scFv).

In some embodiments, the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

In some embodiments, the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain of the CAR further comprises an intracellular signaling domain of a protein selected from the group consisting of a CD3 zeta chain (CD3ζ), FcTRIII, FesRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular domain of the CAR further comprises a costimulatory domain of a CD28, a costimulatory domain of a 4-1BB, an intracellular signaling domain of a CD3 zeta, or any combination thereof.

In some embodiments, the isolated nucleic acid of the invention further comprises a hinge domain.

In some embodiments, the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

In some embodiments, the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

In some embodiments, the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

In another aspect, there is provided a vector comprising the isolated nucleic acid of the invention.

In some embodiments, the vector is a retroviral vector or a lentiviral vector.

In another aspect, there is provided a modified cell, wherein the cell is an immune cell or precursor cell thereof, and wherein the cell is engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

In some embodiments, the tumor antigen is selected from mesothelin, GD2, HER2, TnMuc1, CD70, PMSA, and EGFRvIII.

In some embodiments, the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

In some embodiments, the tumor antigen binding domain is a single-chain variable fragment (scFv).

In some embodiments, the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

In some embodiments, the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular domain of the CAR further comprises a costimulatory domain of a CD28, a costimulatory domain of a 4-1BB, an intracellular signaling domain of a CD3 zeta, or any combination thereof.

In some embodiments, the modified cell of the invention further comprising a hinge domain.

In some embodiments, the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

In some embodiments, the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

In some embodiments, the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

In some embodiments, the cell is a T cell, an autologous cell, a human cell, or any combination thereof.

In some embodiments, the cell is capable of activating STAT1, STAT3, STAT5, or any combination thereof.

In another aspect, there is provided a pharmaceutical composition comprising a population of the modified cell of any one of the preceding embodiments and at least one pharmaceutically acceptable carrier.

In another aspect, there is provided a method of treating cancer in a subject in need thereof, the method comprising administering to the subject a population of modified cells, wherein the cells are immune cells or precursor cells thereof, and wherein the cells are engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

In some embodiments, the tumor antigen is selected from mesothelin, GD2, HER2, TnMuc1, CD70, PMSA, and EGFRvIII.

In some embodiments, the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

In some embodiments, the tumor antigen binding domain is a single-chain variable fragment (scFv).

In some embodiments, the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

In some embodiments, the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular domain of the CAR further comprises a costimulatory domain of a CD28, a costimulatory domain of a 4-1BB, an intracellular signaling domain of a CD3 zeta, or any combination thereof.

In some embodiments of the method of the invention, the CAR further comprises a hinge domain.

In some embodiments, the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

In some embodiments, the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

In some embodiments, the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

In some embodiments of the method of the invention, the population of cells comprises T cells, autologous cells, human cells, or any combination thereof.

In some embodiments, the population of cells is capable of activating STAT1, STAT3, STAT5, or any combination thereof.

In some embodiments, the subject is a human.

In some embodiments, the cancer is selected from a B-cell malignancy (such as a B-cell lymphomas or leukemia), lung cancer, non-small cell lung cancer, small cell lung cancer, Merkel cell carcinoma, melanoma, Hodgkin's lymphoma, non-Hodgkin's lymphoma, urothelial carcinoma, gastric cancer, cervical cancer, cutaneous squamous cell carcinoma, renal cell carcinoma, breast cancer, triple-negative breast cancer, colon cancer, esophagus cancer, stomach cancer, liver cancer, kidney cancer, pancreatic cancer, prostate cancer, brain cancer, lung adenocarcinoma, glioblastoma, hepatocellular carcinoma, gallbladder cancer, cervical cancer, cervical squamous cell carcinoma, colorectal cancer, ovarian cancer, and renal cancer.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A is a schematic illustrating a chimeric antigen receptor (CAR) comprising an antigen-binding scFv extracellular domain, a CD8 hinge domain, a transmembrane domain, and an intracellular domain comprising a 4-1BB and/or CD28 costimulatory domain(s), an IL9Ra signaling domain, and a CD3z signaling domain.

FIG. 1B provides flow cytometry data illustrating expression of CAR on transduced murine T cells compared to untransduced (UTD) cells. The CAR comprises an anti-murine mesothelin (anti-mMSLN) A03 scFv extracellular domain, a murine CD8 hinge and transmembrane domain, and an intracellular domain comprising a murine 4-1BB costimulatory domain, a murine IL9Ra signaling domain, and a murine CD3z signaling domain.

FIG. 2A-FIG. 2D provide data relating to a wild type murine IL9Ra cytokine receptor co-expressed with an exemplary CAR on transduced murine CD3+ T cells. FIG. 3A provides data illustrating co-expression of murine IL9Ra and a murine CAR on transduced murine CD3+ T cells compared to untransduced (UTD) cells. FIG. 2B provides flow cytometry analysis of surface markers CD44, C62L, and Fas (CD95) illustrating the finding that the transduced cells display Tscm phenotype 24 h after stimulation with 100 ng/mL of wild type mIL9 or wild type mIL2. FIG. 2C provides global gene expression profile data in transduced murine CAR T cells expressing mIL9Ra 24 h after stimulation with wild type mIL9 or wild type mIL2. Total RNA was extracted from transduced T cells cultured in the presence of mIL-2 or mIL-9 for 24 h. RNA was analyzed with Nanostring nCounter Mouse Immunology Panel (562 genes) and plotted using nSolver 4.0 software. FIG. 2D provides a graph illustrating in vitro expression of mIL9 via adenoviral vector construct Ad-mIL9. Murine pancreatic cancer cell line PDA7940b (10,000 cells/well) was infected with 100 viral particles/cell of Ad-mIL9 and cell culture supernatants were analyzed for mIL-9 by ELISA at indicated time points.

FIG. 3A provides a schematic of a gene expression construct for expressing human IL9Ra and a human anti-mesothelin CAR (M5), and flow cytometry data showing co-expression of the IL9Ra and the CAR in human T cells.

FIG. 3B provides a schematic of a gene expression construct for expressing murine IL9Ra and a murine anti-mesothelin CAR (A03), and flow cytometry data showing co-expression of the IL9Ra and the CAR in murine cells on Day 5 post transduction.

FIG. 4 provides flow cytometry data illustrating the finding that IL9Ra signaling in T cells leads to a Tscm phenotype.

FIG. 5 provides phospho flow cytometry data illustrating the finding that IL9Ra signaling in T induces phosphorylation of STAT1, STAT3, and STAT5. Shown is the log₂ (fold change) of MFI.

FIG. 6 provides quantified cytokine secretion data for the indicated cytokines in murine T cells incubated with IL9. The T cells were transduced to express the A03 CAR (left side of each panel) or the A03 CAR and IL9Ra (right side of each panel).

FIG. 7 illustrates the finding that IL9Ra signaling in murine T cells enhances tumor cell killing.

FIGS. 8A-8C illustrate the finding that IL9a signaling induces similar gene expression profiles in T cells engineered to express the anti-meso CAR and IL9Ra or anti-meso CAR and an orthogonal chimeric cytokine receptor (ortho-IL2Rβ-IL9Ra chimeric cytokine receptor (o9R)).

FIG. 8A shows top 20 up-regulated and down-regulated genes for T cells expressing anti-meso CAR and IL9Ra pre-incubated with either IL9 or IL2. FIG. 8B shows top 20 up-regulated and down-regulated genes for T cells expressing anti-meso CAR and o9R pre-incubated with ortho-IL2 or IL-2. FIG. 8C shows the shared up-regulated and down-regulated genes.

FIGS. 9A-9F show gene set variation analysis (GSVA) and gene set enrichment analysis (GSEA) data for T cells expressing anti-meso CAR and IL9Ra pre-incubated with either IL9 or IL2 and for T cells expressing anti-meso CAR and an ortho-IL2Rβ-IL9Ra chimeric cytokine receptor (o9R) pre-incubated with ortho-IL2 or IL-2. FIG. 9A data compares the pathways significantly enriched in the CAR T cells stimulated with IL9 vs IL2. FIG. 9B provides a table of the enriched pathways together with the GSEA statistics. FIG. 9C provides enrichment plots and analyses for interferon gamma response in T cells expressing anti-meso CAR and IL9Ra pre-incubated with IL9 vs. IL2. FIG. 9D provides enrichment plots and analyses for interferon alpha response in T cells expressing anti-meso CAR and IL9Ra pre-incubated with IL9 vs. IL2. FIG. 9E provides enrichment plots and analyses for interferon gamma response in T cells expressing anti-meso CAR and ortho-IL2Rβ-IL9Ra chimeric cytokine receptor (o9R) pre-incubated with ortho-IL2 vs. IL-2. FIG. 9F provides enrichment plots and analyses for interferon alpha response in T cells expressing anti-meso CAR and ortho-IL2Rβ-IL9Ra chimeric cytokine receptor (o9R) pre-incubated with ortho-IL2 vs. IL-2.

FIGS. 10A-10D relate to establishment of an in vivo syngeneic murine model of PDA. FIG. 10A shows a schematic of the protocol, charts of the tumor volume, and mesothelin expression data for the PDA7940b cells. FIG. 10B shows the experimental plan for dose titration of an adenoviral vector expressing mIL9 (Ad-mIL9) in the syngeneic PDA murine model. FIG. 10C provides transduction efficiency data for the Ad-mIL9. FIG. 10D provides does titration tumor growth data for the indicated conditions in the syngeneic PDA murine model.

### DETAILED DESCRIPTION

The present disclosure provides a CAR comprising an extracellular tumor antigen binding domain, a transmembrane domain, and an intracellular domain comprising an intracellular signaling domain of IL9Ra, and uses thereof to improve CAR cell immunotherapy for treating cancer by (1) exploiting tumor antigens in tumors to enhance immunostimulatory signals in immune cells (e.g., T cells), (2) altering the phenotype of immune cells expressing the CAR, and (3) enabling IL-9 signaling in the immune cells expressing the CAR to improve effector functions in situ. By repurposing IL-9R signaling using a CAR comprising an IL9Ra intracellular signaling domain in T cells, these cells gain new functions through concomitant activation of STAT1, STAT3 and STAT5. Such CAR T cells assume stem cell memory (Tscm) features with improved trafficking and effector function, thereby resulting in improved antitumor activity for hard-to-treat solid tumors.

It is contemplated herein that a receptor (e.g., a CAR) comprising an IL-9R intracellular domain (ICD) of the present disclosure is distinguished from a receptor (e.g., a CAR) comprising an IL-4R ICD, an IL-7R ICD, or an IL-21R ICD because an orthogonal chimeric cytokine receptor comprising the IL-9R ICD resulted in a potent activation (e.g., phosphorylation) of STAT1, STAT3 and STAT5 in T cells expressing the orthogonal chimeric cytokine receptor. See, Kalbasi, et al. Nature, 607: 360-365 (2022). Indeed, CAR T cells expressing the oIL2Rβ-IL9Ra chimeric cytokine receptor assumed characteristics of stem cell memory and effector T cells and exhibited superior anti-tumor efficacy in two recalcitrant syngeneic mouse solid tumor models of melanoma and pancreatic cancer when compared to, for example, a cell expressing an orthogonal receptor comprising the IL-2,ICD. Furthermore, the anti tumor efficacy of a receptor comprising the IL-9R ICD was effective in the absence of conditioning lymphodepletion. In addition, the CAR T cells expressing the orthogonal chimeric cytokine receptor comprising the IL-9 ICD proliferated less than, e.g., a cell expressing an IL-2 ICD.

Accordingly, the present disclosure provides novel CARs, CAR expressing cells (e.g., CAR T cells), and a novel process for engineering CAR T cells with a stem-like phenotype that does not require administration of an orthogonal cytokine or any exogenous cytokine. It is contemplated herein that the cells of the present invention will exhibit superior antitumor activity. The stem-like phenotype in a T cell was demonstrated herein by expressing wild-type IL9Ra together with a CAR, which resulted in activation of STAT1, STAT3, and STAT5 and enrichment for a CD62L⁺ population and higher expression of Fas (CD95) and Sca-1. CD62L⁺ are known for their superior anti-tumour activity in adoptive cell therapy (ACT).

The novelty of the CARs and CAR-expressing cells disclosed herein is heightened by the fact that IL-9 naive T cells are insensitive to IL-9 and T cell development is unimpaired in IL-9-deficient mice. Mouse T cells do not express an IL-9R receptor. Thus, IL-9 may not be a critical natural cytokine in T cell biology. Indeed, IL-9R is naturally expressed by mast cells, memory B cells, innate lymphoid cells and haematopoietic progenitors. Although T cell subsets that produce IL-9 have been described. However, the effects of IL-9R signaling on T cells are not well characterized. The identification of IL-9 (a lesser-known cytokine among the γc cytokine receptor family) unique signaling properties in T cells, such as the unique STAT signalling profile (e.g., potent activation), and the acquisition of features of stem cell memory T (TSCM) cells were surprising and unexpected.

Accordingly, in one aspect, the invention provides a chimeric antigen receptor (CAR) comprising a tumor antigen binding domain, a transmembrane domain, and an intracellular domain comprising an intracellular signaling domain of an interleukin-9 receptor alpha (IL9Ra).

In another aspect, the invention provides an isolated nucleic acid comprising a nucleotide sequence encoding a chimeric antigen receptor (CAR) comprising a tumor antigen binding domain, a transmembrane domain, and an intracellular domain comprising an intracellular signaling domain of an interleukin-9 receptor alpha (IL9Ra).

In another aspect, the invention provides a modified cell, wherein the cell is an immune cell or precursor cell thereof, and wherein the cell is engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

In another aspect, the invention provides a method of treating cancer in a subject in need thereof, comprising administering to the subject a population of modified cells, wherein the cells are immune cells or precursor cells thereof, and wherein the cells are engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

In other aspects, provided herein are related compositions (e.g., pharmaceutical compositions) and kits.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

Methods and techniques using immune cells with chimeric antigen receptors (e.g., CAR T cells) are described in e.g., Ruella, et al., J. Clin. Invest., 126(10):3814-3826 (2016) and Kalos, et al., 3 (95), 95ra73:1-11 (2011), the contents of which are hereby incorporated by reference in their entireties.

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±10%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used herein, may be a human or non-human mammal. Non-human mammals include, for example, livestock and pets, such as ovine, bovine, porcine, canine, feline and murine mammals, as well as simian and non-human primate mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Chimeric Antigen Receptors (CARs)

The present invention provides a modified immune cell or precursor cell thereof (e.g., a modified T cell) engineered to express a chimeric antigen receptor (CAR) comprising an intracellular domain comprising an interleukin-9 receptor alpha (IL9Ra) intracellular signaling domain (IL-9Ra ICD). The CAR further comprises an extracellular tumor antigen binding domain, a transmembrane domain, and an intracellular domain. The extracellular tumor antigen binding domain of the CAR is operably linked to another domain of the CAR, such as a hinge domain, the transmembrane domain, or the intracellular domain, each described elsewhere herein.

The tumor antigen binding domain described herein can be combined with any of the transmembrane domains described herein, any of the intracellular domains or cytoplasmic domains described herein, or any of the other domains described herein that may be included in a CAR of the present invention, such as a hinge domain or a spacer sequence.

The CAR of the present invention may also include a leader sequence as described herein. The CAR of the present invention may also include a hinge domain as described herein. The CAR of the present invention may also include one or more spacer domains or linkers as described herein which may serve to link one domain of the CAR to the next domain.

### Antigen Binding Domain

The antigen binding domain of a CAR is an extracellular region of the CAR for binding to a specific target antigen including proteins, carbohydrates, and glycolipids. The CAR of the invention comprises an antigen binding domain that is capable of binding a tumor antigen. Suitable tumor antigens are known in the art and include, but are not limited to, alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof. In some embodiments, the tumor antigen is selected from mesothelin, GD2, HER2, TnMuc1, GPC2, CD70, PMSA, and EGFRvIII.

The antigen binding domain can include any domain that binds to the antigen (e.g., tumor antigen) and may include, but is not limited to, a monoclonal antibody (mAb), a polyclonal antibody, a synthetic antibody, a human antibody, a humanized antibody, a non-human antibody, a single-domain antibody, a full length antibody or any antigen-binding fragment thereof, a Fab, and a single-chain variable fragment (scFv). In some embodiments, the antigen binding domain comprises an aglycosylated antibody or a fragment thereof or scFv thereof. In some embodiments, the tumor antigen binding domain is an scFv.

As used herein, the term “single-chain variable fragment” or “scFv” is a fusion protein of the variable regions of the heavy (VH) and light (VL) chains of an immunoglobulin (e.g., murine or human) covalently linked to form a VH::VL heterodimer. The variable heavy (VH) and light (VL) chains are either joined directly or joined by a peptide linker, which connects the N-terminus of the VH with the C-terminus of the VL, or the C-terminus of the VH with the N-terminus of the VL. In some embodiments, the antigen binding domain (e.g., tumor antigen binding domain) comprises an scFv having the configuration from N-terminus to C-terminus, VH-linker-VL. In some embodiments, the antigen binding domain comprises an scFv having the configuration from N-terminus to C-terminus, VL-linker-VH or VH-linker-VL. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers. Those of skill in the art would be able to select the appropriate linker sequence for use in the present invention. In one embodiment, an antigen binding domain of the present invention comprises a heavy chain variable region (VH) and a light chain variable region (VL), wherein the VH and VL are separated by a linker sequence.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 August 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In other embodiments, the antigen binding domain comprises an antibody mimetic protein such as, for example, designed ankyrin repeat protein (DARPin), affibody, monobody, (i.e., adnectin), affilin, affimer, affitin, alphabody, avimer, Kunitz domain peptide, or anticalin. Constructs with specific binding affinities can be generated using DARPin libraries e.g., as described in Seeger, et al., Protein Sci., 22:1239-1257 (2013).

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof, or a humanized murine antibody or a fragment thereof.

In certain embodiments, the antigen binding domain comprises a heavy chain variable region that comprises three heavy chain complementarity determining regions (HCDRs) and a light chain variable region that comprises three light chain complementarity determining regions (LCDRs). In certain embodiments, the antigen binding domain comprises a linker.

### Transmembrane Domain

CARs of the present invention may comprise a transmembrane domain that connects the antigen binding domain of the CAR to the intracellular domain of the CAR. The transmembrane domain of the CAR is a region that is capable of spanning the plasma membrane of a cell (e.g., an immune cell or precursor thereof). In some embodiments, the transmembrane domain is interposed between the antigen binding domain and the intracellular domain of a CAR.

In some embodiments, the transmembrane domain is naturally associated with one or more of the domains in the CAR (e.g., CAR comprising an IL-9R ICD). In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR (e.g., CAR comprising an IL-9R ICD) into a cell membrane, e.g., an artificial hydrophobic sequence. Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), ICOS, CD278, Toll-like receptor 1 (TLR1), TLR2, TLR3, TLR4, TLR5, TLR6, TLR7, TLR8, TLR9 or a transmembrane domain derived from a killer immunoglobulin-like receptor (KIR).

In certain embodiments, the transmembrane domain comprises a transmembrane domain of CD8. In certain embodiments, the transmembrane domain of CD8 is a transmembrane domain of CD8a.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in the CAR comprising an TL-9R ICD described herein.

In some embodiments, the transmembrane domain further comprises a hinge region. The CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, the CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). The hinge region can comprise an amino acid sequence of a human IgG1, IgG2, IgG3, or IgG4, hinge region (see, e.g., Yan et al., J. Biol. Chem. (2012) 287: 5891-5897). In one embodiment, the hinge region can comprise an amino acid sequence derived from human CD8, or a variant thereof.

### Intracellular Signaling Domain

The CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (A, 6 and F), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may comprise an intracellular signaling domain of a protein selected from human CD3 zeta chain, FcyRIII, FcsRI, DAP10, DAP12, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, such as any synthetic sequence thereof, that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, IL-9R, IL-21R, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in the CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of the CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in aa CAR of the present disclosure includes a IL-9Ra or IL-21R type signaling chain. In one embodiment, an intracellular signaling domain suitable for use in a CAR of the present disclosure includes an IL-9Ra intracellular domain as described herein. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire molecule. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular domains described herein can be combined with any of the antigen binding domains described herein, any of the transmembrane domains described herein, or any of the other domains described herein that may be included in the CAR.

In certain embodiments, the intracellular domain comprises a costimulatory domain of 4-1BB. In certain embodiments, the intracellular domain comprises an intracellular domain of CD3ζ or a variant thereof. In certain embodiments, the intracellular domain comprises a costimulatory domain of 4-1BB and an intracellular domain of CD3ζ.

### CAR Comprising ICD Comprising an Intracellular Signaling Domain of IL9Ra

In one aspect, the invention provides a CAR comprising an extracellular tumor antigen binding domain, a transmembrane domain, and an intracellular domain comprising an intracellular signaling domain of IL9Ra. The CAR, and modified cells (e.g., immune cells) expressing the CAR, improve CAR cell immunotherapy for treating cancer by (1) exploiting tumor antigens in tumors to enhance immunostimulatory signals in immune cells (e.g., T cells), (2) altering the phenotype of immune cells expressing the CAR, and (3) enabling IL-9 signaling in the immune cells expressing the CAR to improve effector functions in situ.

In some embodiments, the IL9Ra ICD comprises SEQ ID NO: 1, SEQ ID NO: 4, or SEQ ID NO: 8. In some embodiments, the IL9Ra ICD is encoded by SEQ ID NO: 2, SEQ ID NO: 3, SEQ ID NO: 5, SEQ ID NO: 6, SEQ ID NO: 7, SEQ ID NO: 9, or SEQ ID NO: 124.

In various embodiments, the intracellular domain of the CAR may further comprise one or more costimulatory and/or signaling domains described elsewhere herein, e.g., a CD28 and/or 4-1BB costimulatory domain and/or a CD3z stimulatory domain. In some embodiments, the intracellular domain comprises a CD28 costimulatory domain, an intracellular signaling domain of IL9Ra, and a CD3z stimulatory domain. In some embodiments, the intracellular domain comprises a 4-1BB costimulatory domain, an intracellular signaling domain of IL9Ra, and a CD3z stimulatory domain.

In various embodiments, the tumor antigen binding domain comprises a domain selected from an anti-mesothelin antigen binding domain, an anti-GD2 antigen binding domain, an anti-HER2 antigen binding domain, an anti-GPC2 antigen binding domain, an anti-CD19 antigen binding domain, an anti-TnMuc1 antigen binding domain, an anti-CD70 antigen binding domain, an anti-PMSA antigen binding domain, and an EGFRvIII antigen binding domain.

In some embodiments, the CAR comprises an extracellular anti-mesothelin antigen binding domain, a CD8 hinge, a CD28 TM, and an intracellular domain comprising a CD28 costimulatory domain, an IL9Ra ICD, and a CD3z signaling domain.

In some embodiments, the CAR comprises an extracellular anti-mesothelin antigen binding domain, a CD8 hinge, a CD8 TM, and an intracellular domain comprising a 4-1BB costimulatory domain, an IL9Ra ICD, and a CD3z signaling domain.

Tolerable variations of the individual CAR domain sequences (leader, antigen binding domain, hinge, transmembrane, and/or intracellular domains) will be known to those of skill in the art. For example, in certain embodiments the CAR domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known sequence.

Amino acid and nucleotide sequences for certain embodiments of the CAR and domains thereof are described as follows:

Human IL9Ra ICD
(SEQ ID NO: 1)
KLSPRVKRIFYQNVPSPAMFFQPLYSVHNGNFQTWMGAHGAGVLLSQDCAGTPQGALEPCVQEA
TALLTCGPARPWKSVALEEEQEGPGTRLPGNLSSEDVLPAGCTEWRVQTLAYLPQEDWAPTSLT
RPAPPDSEGSRSSSSSSSSNNNNYCALGCYGGWHLSALPGNTQSSGPIPALACGLSCDHQGLET
QQGVAWVLAGHCQRPGLHEDLQGMLLPSVLSKARSWTF
Human IL9Ra ICD
(SEQ ID NO: 2)
AAGCTGAGCCCCAGGGTGAAGAGGATCTTCTACCAGAACGTGCCCAGCCCCGCCATGTTCTTCC
AGCCCCTGTACAGCGTGCACAACGGCAACTTCCAGACCTGGATGGGCGCCCACGGCGCCGGCGT
GCTGCTGAGCCAGGACTGCGCCGGCACCCCCCAGGGCGCCCTGGAGCCCTGCGTGCAGGAGGCC
ACCGCCCTGCTGACCTGCGGCCCCGCCAGGCCCTGGAAGAGCGTGGCCCTGGAGGAGGAGCAGG
AGGGCCCCGGCACCAGGCTGCCCGGCAACCTGAGCAGCGAGGACGTGCTGCCCGCCGGCTGCAC
CGAGTGGAGGGTGCAGACCCTGGCCTACCTGCCCCAGGAGGACTGGGCCCCCACCAGCCTGACC
AGGCCCGCCCCCCCCGACAGCGAGGGCAGCAGGAGCAGCAGCAGCAGCAGCAGCAGCAACAACA
ACAACTACTGCGCCCTGGGCTGCTACGGCGGCTGGCACCTGAGCGCCCTGCCCGGCAACACCCA
GAGCAGCGGCCCCATCCCCGCCCTGGCCTGCGGCCTGAGCTGCGACCACCAGGGCCTGGAGACC
CAGCAGGGCGTGGCCTGGGTGCTGGCCGGCCACTGCCAGAGGCCCGGCCTGCACGAGGACCTGC
AGGGCATGCTGCTGCCCAGCGTGCTGAGCAAGGCCAGGAGCTGGACCTTC
Human IL9Ra ICD
(SEQ ID NO: 3)
AAGCTGAGCCCTAGAGTGAAAAGAATCTTCTACCAGAACGTGCCTTCTCCAGCCATGTTCTTCC
AGCCTCTGTACAGCGTGCACAACGGCAACTTCCAGACCTGGATGGGCGCTCACGGCGCCGGCGT
TCTGCTGAGCCAGGATTGCGCCGGAACACCTCAAGGCGCTCTGGAACCTTGCGTGCAGGAGGCC
ACCGCCCTGCTGACCTGTGGCCCTGCTCGGCCCTGGAAGTCCGTGGCCCTAGAGGAAGAGCAGG
AAGGCCCCGGGACCAGACTGCCTGGCAACCTGAGCAGCGAGGACGTGCTGCCTGCCGGATGTAC
TGAGTGGCGGGTGCAGACCCTGGCCTACCTGCCCCAGGAGGACTGGGCTCCAACATCTCTGACC
AGACCGGCCCCTCCAGACAGCGAAGGCAGCAGATCTAGCAGCAGCAGCAGTTCTTCTAATAACA
ACAACTACTGTGCCCTCGGCTGTTACGGCGGCTGGCACCTGAGCGCCCTCCCTGGAAATACACA
GTCTAGCGGCCCTATCCCCGCCCTGGCTTGTGGACTGAGCTGCGACCACCAGGGCCTGGAAACA
CAGCAGGGCGTGGCCTGGGTCCTGGCCGGCCACTGCCAGAGACCTGGCCTGCACGAGGACCTGC
AGGGAATGCTGCTGCCCAGCGTCCTGAGCAAGGCCAGAAGCTGGACCTTT
Murine IL9Ra ICD
(SEQ ID NO: 4)
KLSPRLKRIFYQNIPSPEAFFHPLYSVYHGDFQSWTGARRAGPQARQNGVSTSSAGSESSIWEA
VATLTYSPACPVQFACLKWEATAPGFPGLPGSEHVLPAGCLELEGQPSAYLPQEDWAPLGSARP
PPPDSDSGSSDYCMLDCCEECHLSAFPGHTESPELTLAQPVALPVSSRA
Murine IL9Ra ICD
(SEQ ID NO: 5)
AAGCTGTCACCCAGGCTGAAGAGAATCTTTTACCAGAACATTCCATCTCCCGAGGCGTTCTTCC
ATCCTCTCTACAGTGTGTACCATGGGGACTTCCAGAGTTGGACAGGGGCCCGCAGAGCCGGACC
ACAAGCAAGACAGAATGGTGTCAGTACTTCATCAGCAGGCTCAGAGTCCAGCATCTGGGAGGCC
GTCGCCACACTCACCTATAGCCCGGCATGCCCTGTGCAGTTTGCCTGCCTGAAGTGGGAGGCCA
CAGCCCCGGGCTTCCCAGGGCTCCCAGGCTCAGAGCATGTGCTGCCGGCAGGGTGTCTGGAGTT
GGAAGGACAGCCATCTGCCTACCTGCCCCAGGAGGACTGGGCCCCACTGGGCTCTGCCAGGCCC
CCTCCTCCAGACTCAGACAGCGGCAGCAGCGACTATTGCATGTTGGACTGCTGTGAGGAATGCC
ACCTCTCAGCCTTCCCAGGACACACCGAGAGTCCTGAGCTCACGCTAGCTCAGCCTGTGGCCCT
TCCTGTGTCCAGCAGGGCC
Murine IL9Ra ICD
(SEQ ID NO: 6)
AAGCTGTCACCTCGCCTTAAACGAATCTTCTACCAGAATATCCCGTCGCCTGAGGCTTTCTTCC
ACCCTCTCTATAGTGTCTACCACGGAGACTTCCAATCGTGGACTGGTGCAAGGCGGGCCGGCCC
ACAAGCCAGGCAAAATGGTGTGTCAACAAGCAGCGCAGGCAGTGAGTCTTCCATCTGGGAAGCG
GTCGCCACCTTGACTTACAGTCCAGCTTGTCCAGTCCAGTTTGCCTGCCTAAAGTGGGAGGCAA
CAGCCCCCGGATTCCCCGGTCTGCCCGGCAGTGAACATGTACTACCTGCAGGATGTTTGGAGTT
AGAAGGCCAACCTTCAGCATACTTGCCTCAGGAAGACTGGGCTCCACTGGGCTCTGCTAGACCA
CCCCCTCCAGACTCCGACTCAGGGTCCTCAGACTACTGCATGCTGGACTGCTGTGAGGAGTGTC
ACCTGAGCGCTTTCCCTGGGCACACTGAATCTCCAGAACTGACTCTGGCCCAGCCCGTGGCCCT
GCCAGTATCAAGCCGAGCC
Murine IL9Ra ICD
(SEQ ID NO: 7)
AAGCTGAGCCCCAGGCTGAAGAGGATCTTCTACCAGAACATCCCCAGCCCCGAGGCCTTCTTCC
ACCCCCTGTACAGCGTGTACCACGGCGACTTCCAGAGCTGGACCGGCGCCAGGAGGGCCGGCCC
CCAGGCCAGGCAGAACGGCGTGAGCACCAGCAGCGCCGGCAGCGAGAGCAGCATCTGGGAGGCC
GTGGCCACCCTGACCTACAGCCCCGCCTGCCCCGTGCAGTTCGCCTGCCTGAAGTGGGAGGCCA
CCGCCCCCGGCTTCCCCGGCCTGCCCGGCAGCGAGCACGTGCTGCCCGCCGGCTGCCTGGAGCT
GGAGGGCCAGCCCAGCGCCTACCTGCCCCAGGAGGACTGGGCCCCCCTGGGCAGCGCCAGGCCC
CCCCCCCCCGACAGCGACAGCGGCAGCAGCGACTACTGCATGCTGGACTGCTGCGAGGAGTGCC
ACCTGAGCGCCTTCCCCGGCCACACCGAGAGCCCCGAGCTGACCCTGGCCCAGCCCGTGGCCCT
GCCCGTGAGCAGCAGGGCC
Murine IL9Ra ICD
(SEQ ID NO: 8)
KLSPRLKRIFYQNIPSPEAFFHPLYSVYHGDFQSWTGARRAGPQARQNGVSTSSAGSESSIWEA
VATLTYSPACPVQFACLKWEATAPGFPGLPGSEHVLPAGCLELEGQPSAYLPQEDWAPLGSARP
PPPDSDSGSSDYCMLDCCEECHLSAFPGHTESPELTLAQPVALPVSSRA
Murine IL9Ra ICD
(SEQ ID NO: 9)
AAGCTGAGCCCCAGGCTGAAGAGGATCTTCTACCAGAACATCCCCAGCCCCGAGGCCTTCTTCC
ACCCCCTGTACAGCGTGTACCACGGCGACTTCCAGAGCTGGACCGGCGCCAGGAGGGCCGGCCC
CCAGGCCAGGCAGAACGGCGTGAGCACCAGCAGCGCCGGCAGCGAGAGCAGCATCTGGGAGGCC
GTGGCCACCCTGACCTACAGCCCCGCCTGCCCCGTGCAGTTCGCCTGCCTGAAGTGGGAGGCCA
CCGCCCCCGGCTTCCCCGGCCTGCCCGGCAGCGAGCACGTGCTGCCCGCCGGCTGCCTGGAGCT
GGAGGGCCAGCCCAGCGCCTACCTGCCCCAGGAGGACTGGGCCCCCCTGGGCAGCGCCAGGCCC
CCCCCCCCCGACAGCGACAGCGGCAGCAGCGACTACTGCATGCTGGACTGCTGCGAGGAGTGCC
ACCTGAGCGCCTTCCCCGGCCACACCGAGAGCCCCGAGCTGACCCTGGCCCAGCCCGTGGCCCT
GCCCGTGAGCAGCAGGGCC
Anti-human mesothelin (anti-hMSLN) scFv (M5)
(SEQ ID NO: 49)
MALPVTALLLPLALLLHAARPQVQLVQSGAEVEKPGASVKVSCKASGYTFTDYYMHWVRQAPGQ
GLEWMGWINPNSGGTNYAQKFQGRVTMTRDTSISTAYMELSRLRSDDTAVYYCASGWDFDYWGQ
GTLVTVSSGGGGSGGGGSGGGGSGGGGSDIVMTQSPSSLSASVGDRVTITCRASQSIRYYLSWY
QQKPGKAPKLLIYTASILQNGVPSRFSGSGSGTDFTLTISSLQPEDFATYYCLQTYTTPDFGPG
TKVEIK
Anti-human mesothelin (anti-hMSLN) scFv (M5)
(SEQ ID NO: 50)
ATGGCCCTGCCCGTGACCGCCCTGCTGCTGCCCCTGGCCCTGCTGCTGCACGCCGCCAGGCCCC
AGGTGCAGCTGGTGCAGAGCGGCGCCGAGGTGGAGAAGCCCGGCGCCAGCGTGAAGGTGAGCTG
CAAGGCCAGCGGCTACACCTTCACCGACTACTACATGCACTGGGTGAGGCAGGCCCCCGGCCAG
GGCCTGGAGTGGATGGGCTGGATCAACCCCAACAGCGGCGGCACCAACTACGCCCAGAAGTTCC
AGGGCAGGGTGACCATGACCAGGGACACCAGCATCAGCACCGCCTACATGGAGCTGAGCAGGCT
GAGGAGCGACGACACCGCCGTGTACTACTGCGCCAGCGGCTGGGACTTCGACTACTGGGGCCAG
GGCACCCTGGTGACCGTGAGCAGCGGCGGCGGCGGCAGCGGCGGCGGCGGCAGCGGCGGCGGCG
GCAGCGGCGGCGGCGGCAGCGACATCGTGATGACCCAGAGCCCCAGCAGCCTGAGCGCCAGCGT
GGGCGACAGGGTGACCATCACCTGCAGGGCCAGCCAGAGCATCAGGTACTACCTGAGCTGGTAC
CAGCAGAAGCCCGGCAAGGCCCCCAAGCTGCTGATCTACACCGCCAGCATCCTGCAGAACGGCG
TGCCCAGCAGGTTCAGCGGCAGCGGCAGCGGCACCGACTTCACCCTGACCATCAGCAGCCTGCA
GCCCGAGGACTTCGCCACCTACTACTGCCTGCAGACCTACACCACCCCCGACTTCGGCCCCGGC
ACCAAGGTGGAGATCAAG
Human CD8 Hinge
(SEQ ID NO: 51)
TTTPAPRPPTPAPTIASQPLSLRPEACRPAAGGAVHTRGLDFACD
Human CD8 Hinge
(SEQ ID NO: 52)
ACCACCACCCCCGCCCCCAGGCCCCCCACCCCCGCCCCCACCATCGCCAGCCAGCCCCTGAGCC
TGAGGCCCGAGGCCTGCAGGCCCGCCGCCGGCGGCGCCGTGCACACCAGGGGCCTGGACTTCGC
CTGCGAC
Human CD8 TM
(SEQ ID NO: 53)
IYIWAPLAGTCGVLLLSLVITLYC
Human CD8 TM
(SEQ ID NO: 54)
ATCTACATCTGGGCCCCCCTGGCCGGCACCTGCGGCGTGCTGCTGCTGAGCCTGGTGATCACCC
TGTACTGC
Human CD8 Hinge and TM
(SEQ ID NO: 55)
TTTPAPRPPTPAPTIASQPLSLRPEACRPAAGGAVHTRGLDFACDIYIWAPLAGTCGVLLLSLV
ITLYC
Human CD8 Hinge and TM
(SEQ ID NO: 56)
ACCACCACCCCCGCCCCCAGGCCCCCCACCCCCGCCCCCACCATCGCCAGCCAGCCCCTGAGCC
TGAGGCCCGAGGCCTGCAGGCCCGCCGCCGGCGGCGCCGTGCACACCAGGGGCCTGGACTTCGC
CTGCGACATCTACATCTGGGCCCCCCTGGCCGGCACCTGCGGCGTGCTGCTGCTGAGCCTGGTG
ATCACCCTGTACTGC
Human CD28 TM
(SEQ ID NO: 57)
FWVLVVVGGVLACYSLLVTVAFIIFWV
Human CD28 TM
(SEQ ID NO: 58)
TTCTGGGTGCTGGTGGTGGTGGGCGGCGTGCTGGCCTGCTACAGCCTGCTGGTGACCGTGGCCT
TCATCATCTTCTGGGTG
Human CD28 costimulatory domain (hCD28 ICD)
(SEQ ID NO: 59)
RSKRSRLLHSDYMNMTPRRPGPTRKHYQPYAPPRDFAAYRS
Human CD28 costimulatory domain (hCD28 ICD)
(SEQ ID NO: 60)
AGGAGCAAGAGGAGCAGGCTGCTGCACAGCGACTACATGAACATGACCCCCAGGAGGCCCGGCC
CCACCAGGAAGCACTACCAGCCCTACGCCCCCCCCAGGGACTTCGCCGCCTACAGGAGC
Human 4-1BB costimulatory domain (h41BB ICD)
(SEQ ID NO: 61)
KRGRKKLLYIFKQPFMRPVQTTQEEDGCSCRFPEEEEGGCEL
Human 4-1BB costimulatory domain (h41BB ICD)
(SEQ ID NO: 62)
AAGAGGGGCAGGAAGAAGCTGCTGTACATCTTCAAGCAGCCCTTCATGAGGCCCGTGCAGACCA
CCCAGGAGGAGGACGGCTGCAGCTGCAGGTTCCCCGAGGAGGAGGAGGGCGGCTGCGAGCTG
Human CD3z stimulatory domain (hCD3z ICD)
(SEQ ID NO: 63)
RVKFSRSADAPAYKQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
Human CD3z stimulatory domain (hCD3z ICD)
(SEQ ID NO: 64)
AGGGTGAAGTTCAGCAGGAGCGCCGACGCCCCCGCCTACAAGCAGGGCCAGAACCAGCTGTACA
ACGAGCTGAACCTGGGCAGGAGGGAGGAGTACGACGTGCTGGACAAGAGGAGGGGCAGGGACCC
CGAGATGGGCGGCAAGCCCAGGAGGAAGAACCCCCAGGAGGGCCTGTACAACGAGCTGCAGAAG
GACAAGATGGCCGAGGCCTACAGCGAGATCGGCATGAAGGGCGAGAGGAGGAGGGGCAAGGGCC
ACGACGGCCTGTACCAGGGCCTGAGCACCGCCACCAAGGACACCTACGACGCCCTGCACATGCA
GGCCCTGCCCCCCAGG
Anti-murine mesothelin (anti-mMSLN) scFv (A03)
(SEQ ID NO: 65)
MASPLTRFLSLNLLLLGESIILGSGEATRAQVQLQESGPGLVKPSQTLSLTCTVSGGSISSGGY
YWSWIRQHPGKGLEWIGYIYYSGSTYYNPSLKSRVTISVDTSKNQFSLKLSSVTAADTAVYYCA
RFDYGDFYDAFDIWGQGTMVTVSSGGGGSGGGGSGGGGSEIVLTQSPSSLSASVGDRVTITCRA
SQGISSALAWYQQKPGKAPKLLIYDASSLESGVPSRFSGSGSGTDFTLTISSLQPEDFATYYCQ
QFNSYPITFGQGTRLEIKRSG
Anti-murine mesothelin (anti-mMSLN) scFv (A03)
(SEQ ID NO: 66)
ATGGCTAGTCCGCTCACGAGGTTTTTGTCTCTGAACCTTCTCTTATTAGGGGAAAGCATAATCC
TGGGCAGCGGCGAGGCTACGCGGGCGCAGGTTCAGCTGCAAGAGTCCGGACCCGGTCTGGTGAA
GCCCAGCCAGACTTTGAGCCTGACCTGTACCGTATCTGGTGGCTCCATAAGTTCTGGAGGCTAC
TACTGGAGCTGGATAAGGCAGCACCCAGGGAAGGGCCTGGAGTGGATCGGCTATATTTACTACA
GCGGGAGCACTTATTATAATCCCTCATTAAAGAGCAGGGTCACCATCTCAGTGGACACATCCAA
GAACCAGTTCAGCTTGAAACTCTCTTCCGTAACAGCTGCTGACACTGCCGTTTACTATTGTGCC
AGGTTTGACTACGGAGATTTTTACGATGCCTTTGATATATGGGGCCAAGGCACCATGGTGACAG
TCTCCTCAGGTGGAGGAGGCAGTGGGGGGGGGGGGTCTGGGGGTGGTGGCTCTGAGATCGTTCT
AACCCAGAGCCCGAGCAGCCTATCGGCGTCAGTGGGAGATAGAGTGACCATTACCTGCAGGGCA
AGTCAAGGCATAAGCAGCGCTCTGGCCTGGTACCAACAAAAGCCTGGAAAGGCTCCTAAGCTGC
TGATTTATGATGCTTCGAGTCTCGAAAGTGGTGTCCCGTCAAGGTTTTCTGGTAGTGGTTCAGG
TACAGACTTCACCTTGACTATCAGCTCGCTCCAACCAGAAGATTTCGCAACATATTACTGCCAG
CAGTTCAACAGCTACCCCATTACATTTGGACAAGGAACCCGGCTTGAAATTAAACGCTCAGGG
Murine CD8 Hinge
(SEQ ID NO: 67)
LQKVNSTTTKPVLRTPSPVHPTGTSQPQRPEDCRPRGSVKGTGLDFACDIY
Murine CD8 Hinge
(SEQ ID NO: 68)
CTTCAGAAGGTGAACAGCACAACAACCAAGCCAGTCTTGCGAACACCCAGTCCTGTTCACCCTA
CGGGTACGTCTCAACCTCAGAGGCCTGAGGACTGTAGACCCCGTGGCTCTGTGAAAGGGACAGG
GCTGGACTTTGCTTGTGACATCTAC
Murine CD8 TM
(SEQ ID NO: 69)
IWAPLAGICVALLLSLIITLI
Murine CD8 TM
(SEQ ID NO: 70)
ATCTGGGCACCCTTAGCCGGTATCTGTGTGGCCTTGCTGCTTTCCCTCATCATCACTCTAATT
Murine CD8 Hinge and TM
(SEQ ID NO: 71)
LQKVNSTTTKPVLRTPSPVHPTGTSQPQRPEDCRPRGSVKGTGLDFACDIYTWAPLAGICVALL
LSLITTLI
Murine CD8 Hinge and TM
(SEQ ID NO: 72)
CTTCAGAAGGTGAACAGCACAACAACCAAGCCAGTCTTGCGAACACCCAGTCCTGTTCACCCTA
CGGGTACGTCTCAACCTCAGAGGCCTGAGGACTGTAGACCCCGTGGCTCTGTGAAAGGGACAGG
GCTGGACTTTGCTTGTGACATCTACATCTGGGCACCCTTAGCCGGTATCTGTGTGGCCTTGCTG
CTTTCCCTCATCATCACTCTAATT
Murine CD28 TM
(SEQ ID NO: 73)
FWALVVVAGVLFCYGLLVTVALCVIWT
Murine CD28 TM
(SEQ ID NO: 74)
TTCTGGGCCCTGGTGGTGGTGGCCGGCGTGCTGTTCTGCTACGGCCTGCTGGTGACCGTGGCCC
TGTGCGTGATCTGGACC
Murine CD28 costimulatory domain (mCD28 ICD)
(SEQ ID NO: 75)
NSRRNRLLQSDYMNMTPRRPGLTRKPYQPYAPARDFAAYRP
Murine CD28 costimulatory domain (mCD28 ICD)
(SEQ ID NO: 76)
AACAGCAGGAGGAACAGGCTGCTGCAGAGCGACTACATGAACATGACCCCCAGGAGGCCCGGCC
TGACCAGGAAGCCCTACCAGCCCTACGCCCCCGCCAGGGACTTCGCCGCCTACAGGCCC
Murine 4-IBB costimulatory domain (m41BB ICD)
(SEQ ID NO: 77)
KWIRKKFPHIFKQPFKKTTGAAQEEDACSCRCPQEEEGGGGGYEL
Murine 4-IBB costimulatory domain (m41BB ICD)
(SEQ ID NO: 78)
AAGTGGATTCGAAAAAAGTTCCCCCACATCTTTAAGCAGCCGTTCAAGAAAACCACTGGAGCAG
CCCAGGAGGAGGATGCTTGCAGCTGCCGCTGTCCCCAGGAGGAAGAAGGCGGCGGGGGCGGATA
TGAGCTC
Murine CD3z stimulatory domain (mCD3z ICD)
(SEQ ID NO: 79)
KFSRSAETAANLQDPNQLYNELNLGRREEYDVLEKKRARDPEMGGKQQRRRNPQEGVYNALQKD
KMAEAYSEIGTKGERRRGKGHDGLYQGLSTATKDTYDALHMQTLAPR
Murine CD3z stimulatory domain (mCD3z ICD)
(SEQ ID NO: 80)
AAGTTTTCACGCTCTGCAGAGACAGCTGCCAACCTGCAGGACCCCAATCAGCTGTACAATGAAC
TGAATCTCGGGCGGAGAGAAGAATATGATGTGTTGGAGAAGAAGCGTGCGAGAGACCCAGAGAT
GGGCGGCAAACAGCAGAGAAGACGAAACCCACAGGAAGGAGTGTACAACGCCCTGCAGAAAGAC
AAGATGGCAGAGGCCTACTCAGAGATTGGAACCAAAGGAGAGAGGCGCCGTGGAAAAGGACATG
ATGGGCTTTACCAGGGTTTAAGTACGGCCACTAAAGATACTTATGACGCGCTGCACATGCAGAC
ACTGGCACCTCGA
Human anti-hMSLN scFv - hCD8 hinge - hCD28 TM and ICD - hIL9Ra ICD - hCD3z
(SEQ ID NO: 81)
MALPVTALLLPLALLLHAARPQVQLVQSGAEVEKPGASVKVSCKASGYTFTDYYMHWVRQAPGQ
GLEWMGWINPNSGGTNYAQKFQGRVTMTRDTSISTAYMELSRLRSDDTAVYYCASGWDFDYWGQ
GTLVTVSSGGGGSGGGGSGGGGSGGGGSDIVMTQSPSSLSASVGDRVTITCRASQSIRYYLSWY
QQKPGKAPKLLIYTASILQNGVPSRFSGSGSGTDFTLTISSLQPEDFATYYCLQTYTTPDFGPG
TKVEIKTTTPAPRPPTPAPTIASQPLSLRPEACRPAAGGAVHTRGLDFACDFWVLVVVGGVLAC
YSLLVTVAFIIFWVRSKRSRLLHSDYMNMTPRRPGPTRKHYQPYAPPRDFAAYRSKLSPRVKRI
FYQNVPSPAMFFQPLYSVHNGNFQTWMGAHGAGVLLSQDCAGTPQGALEPCVQEATALLTCGPA
RPWKSVALEEEQEGPGTRLPGNLSSEDVLPAGCTEWRVQTLAYLPQEDWAPTSLTRPAPPDSEG
SRSSSSSSSSNNNNYCALGCYGGWHLSALPGNTQSSGPIPALACGLSCDHQGLETQQGVAWVLA
GHCQRPGLHEDLQGMLLPSVLSKARSWTFRVKFSRSADAPAYKQGQNQLYNELNLGRREEYDVL
DKRRGRDPEMGGKPRRKNPQEGLYNELQKDKMAEAYSEIGMKGERRRGKGHDGLYQGLSTATKD
TYDALHMQALPPR
Human anti-hMSLN scFv - hCD8 hinge - hCD28 TM and ICD - hIL9Ra ICD - hCD3z
(SEQ ID NO: 82)
ATGGCCCTGCCCGTGACCGCCCTGCTGCTGCCCCTGGCCCTGCTGCTGCACGCCGCCAGGCCCC
AGGTGCAGCTGGTGCAGAGCGGCGCCGAGGTGGAGAAGCCCGGCGCCAGCGTGAAGGTGAGCTG
CAAGGCCAGCGGCTACACCTTCACCGACTACTACATGCACTGGGTGAGGCAGGCCCCCGGCCAG
GGCCTGGAGTGGATGGGCTGGATCAACCCCAACAGCGGCGGCACCAACTACGCCCAGAAGTTCC
AGGGCAGGGTGACCATGACCAGGGACACCAGCATCAGCACCGCCTACATGGAGCTGAGCAGGCT
GAGGAGCGACGACACCGCCGTGTACTACTGCGCCAGCGGCTGGGACTTCGACTACTGGGGCCAG
GGCACCCTGGTGACCGTGAGCAGCGGCGGCGGCGGCAGCGGCGGCGGCGGCAGCGGCGGCGGCG
GCAGCGGCGGCGGCGGCAGCGACATCGTGATGACCCAGAGCCCCAGCAGCCTGAGCGCCAGCGT
GGGCGACAGGGTGACCATCACCTGCAGGGCCAGCCAGAGCATCAGGTACTACCTGAGCTGGTAC
CAGCAGAAGCCCGGCAAGGCCCCCAAGCTGCTGATCTACACCGCCAGCATCCTGCAGAACGGCG
TGCCCAGCAGGTTCAGCGGCAGCGGCAGCGGCACCGACTTCACCCTGACCATCAGCAGCCTGCA
GCCCGAGGACTTCGCCACCTACTACTGCCTGCAGACCTACACCACCCCCGACTTCGGCCCCGGC
ACCAAGGTGGAGATCAAGACCACCACCCCCGCCCCCAGGCCCCCCACCCCCGCCCCCACCATCG
CCAGCCAGCCCCTGAGCCTGAGGCCCGAGGCCTGCAGGCCCGCCGCCGGCGGCGCCGTGCACAC
CAGGGGCCTGGACTTCGCCTGCGACTTCTGGGTGCTGGTGGTGGTGGGCGGCGTGCTGGCCTGC
TACAGCCTGCTGGTGACCGTGGCCTTCATCATCTTCTGGGTGAGGAGCAAGAGGAGCAGGCTGC
TGCACAGCGACTACATGAACATGACCCCCAGGAGGCCCGGCCCCACCAGGAAGCACTACCAGCC
CTACGCCCCCCCCAGGGACTTCGCCGCCTACAGGAGCAAGCTGAGCCCCAGGGTGAAGAGGATC
TTCTACCAGAACGTGCCCAGCCCCGCCATGTTCTTCCAGCCCCTGTACAGCGTGCACAACGGCA
ACTTCCAGACCTGGATGGGCGCCCACGGCGCCGGCGTGCTGCTGAGCCAGGACTGCGCCGGCAC
CCCCCAGGGCGCCCTGGAGCCCTGCGTGCAGGAGGCCACCGCCCTGCTGACCTGCGGCCCCGCC
AGGCCCTGGAAGAGCGTGGCCCTGGAGGAGGAGCAGGAGGGCCCCGGCACCAGGCTGCCCGGCA
ACCTGAGCAGCGAGGACGTGCTGCCCGCCGGCTGCACCGAGTGGAGGGTGCAGACCCTGGCCTA
CCTGCCCCAGGAGGACTGGGCCCCCACCAGCCTGACCAGGCCCGCCCCCCCCGACAGCGAGGGC
AGCAGGAGCAGCAGCAGCAGCAGCAGCAGCAACAACAACAACTACTGCGCCCTGGGCTGCTACG
GCGGCTGGCACCTGAGCGCCCTGCCCGGCAACACCCAGAGCAGCGGCCCCATCCCCGCCCTGGC
CTGCGGCCTGAGCTGCGACCACCAGGGCCTGGAGACCCAGCAGGGCGTGGCCTGGGTGCTGGCC
GGCCACTGCCAGAGGCCCGGCCTGCACGAGGACCTGCAGGGCATGCTGCTGCCCAGCGTGCTGA
GCAAGGCCAGGAGCTGGACCTTCAGGGTGAAGTTCAGCAGGAGCGCCGACGCCCCCGCCTACAA
GCAGGGCCAGAACCAGCTGTACAACGAGCTGAACCTGGGCAGGAGGGAGGAGTACGACGTGCTG
GACAAGAGGAGGGGCAGGGACCCCGAGATGGGCGGCAAGCCCAGGAGGAAGAACCCCCAGGAGG
GCCTGTACAACGAGCTGCAGAAGGACAAGATGGCCGAGGCCTACAGCGAGATCGGCATGAAGGG
CGAGAGGAGGAGGGGCAAGGGCCACGACGGCCTGTACCAGGGCCTGAGCACCGCCACCAAGGAC
ACCTACGACGCCCTGCACATGCAGGCCCTGCCCCCCAGG
Human anti-hMSLN scFv - hCD8 hinge and TM - h41BB ICD - hIL9Ra ICD - hCD3z
(SEQ ID NO: 83)
MALPVTALLLPLALLLHAARPQVQLVQSGAEVEKPGASVKVSCKASGYTFTDYYMHWVRQAPGQ
GLEWMGWINPNSGGTNYAQKFQGRVTMTRDTSISTAYMELSRLRSDDTAVYYCASGWDFDYWGQ
GTLVTVSSGGGGSGGGGSGGGGSGGGGSDIVMTQSPSSLSASVGDRVTITCRASQSIRYYLSWY
QQKPGKAPKLLIYTASILQNGVPSRFSGSGSGTDFTLTISSLQPEDFATYYCLQTYTTPDFGPG
TKVEIKTTTPAPRPPTPAPTIASQPLSLRPEACRPAAGGAVHTRGLDFACDIYIWAPLAGTCGV
EEESEVIYEYCKRGRKKLLYIFKQPFMRPVQTTQEEDGCSCRFPEEEEGGCELKLSPRYKRIFY
QNVPSPAMFFQPLYSVHNGNFQTWMGAHGAGVLLSQDCAGTPQGALEPCVQEATALLTCGPARP
WKSVALEEEQEGPGTRLPGNLSSEDVLPAGCTEWRVQTLAYLPQEDWAPTSLTRPAPPDSEGSR
SSSSSSSSNNNNYCALGCYGGWHLSALPGNTQSSGPIPALACGLSCDHQGLETQQGVAWVLAGH
CQRPGLHEDLQGMLLPSVLSKARSWTFRVKFSRSADAPAYKQGQNQLYNELNLGRREEYDVLDK
RRGRDPEMGGKPRRKNPQEGLYNELQKDKMAEAYSEIGMKGERRRGKGHDGLYQGLSTATKDTY
DALHMQALPPR
Human anti-hMSLN scFv - hCD8 hinge and TM - h41BB ICD - hIL9Ra ICD - hCD3z
(SEQ ID NO: 84)
ATGGCCCTGCCCGTGACCGCCCTGCTGCTGCCCCTGGCCCTGCTGCTGCACGCCGCCAGGCCCC
AGGTGCAGCTGGTGCAGAGCGGCGCCGAGGTGGAGAAGCCCGGCGCCAGCGTGAAGGTGAGCTG
CAAGGCCAGCGGCTACACCTTCACCGACTACTACATGCACTGGGTGAGGCAGGCCCCCGGCCAG
GGCCTGGAGTGGATGGGCTGGATCAACCCCAACAGCGGCGGCACCAACTACGCCCAGAAGTTCC
AGGGCAGGGTGACCATGACCAGGGACACCAGCATCAGCACCGCCTACATGGAGCTGAGCAGGCT
GAGGAGCGACGACACCGCCGTGTACTACTGCGCCAGCGGCTGGGACTTCGACTACTGGGGCCAG
GGCACCCTGGTGACCGTGAGCAGCGGCGGCGGCGGCAGCGGCGGCGGCGGCAGCGGCGGCGGCG
GCAGCGGCGGCGGCGGCAGCGACATCGTGATGACCCAGAGCCCCAGCAGCCTGAGCGCCAGCGT
GGGCGACAGGGTGACCATCACCTGCAGGGCCAGCCAGAGCATCAGGTACTACCTGAGCTGGTAC
CAGCAGAAGCCCGGCAAGGCCCCCAAGCTGCTGATCTACACCGCCAGCATCCTGCAGAACGGCG
TGCCCAGCAGGTTCAGCGGCAGCGGCAGCGGCACCGACTTCACCCTGACCATCAGCAGCCTGCA
GCCCGAGGACTTCGCCACCTACTACTGCCTGCAGACCTACACCACCCCCGACTTCGGCCCCGGC
ACCAAGGTGGAGATCAAGACCACCACCCCCGCCCCCAGGCCCCCCACCCCCGCCCCCACCATCG
CCAGCCAGCCCCTGAGCCTGAGGCCCGAGGCCTGCAGGCCCGCCGCCGGCGGCGCCGTGCACAC
CAGGGGCCTGGACTTCGCCTGCGACATCTACATCTGGGCCCCCCTGGCCGGCACCTGCGGCGTG
CTGCTGCTGAGCCTGGTGATCACCCTGTACTGCAAGAGGGGCAGGAAGAAGCTGCTGTACATCT
TCAAGCAGCCCTTCATGAGGCCCGTGCAGACCACCCAGGAGGAGGACGGCTGCAGCTGCAGGTT
CCCCGAGGAGGAGGAGGGCGGCTGCGAGCTGAAGCTGAGCCCCAGGGTGAAGAGGATCTTCTAC
CAGAACGTGCCCAGCCCCGCCATGTTCTTCCAGCCCCTGTACAGCGTGCACAACGGCAACTTCC
AGACCTGGATGGGCGCCCACGGCGCCGGCGTGCTGCTGAGCCAGGACTGCGCCGGCACCCCCCA
GGGCGCCCTGGAGCCCTGCGTGCAGGAGGCCACCGCCCTGCTGACCTGCGGCCCCGCCAGGCCC
TGGAAGAGCGTGGCCCTGGAGGAGGAGCAGGAGGGCCCCGGCACCAGGCTGCCCGGCAACCTGA
GCAGCGAGGACGTGCTGCCCGCCGGCTGCACCGAGTGGAGGGTGCAGACCCTGGCCTACCTGCC
CCAGGAGGACTGGGCCCCCACCAGCCTGACCAGGCCCGCCCCCCCCGACAGCGAGGGCAGCAGG
AGCAGCAGCAGCAGCAGCAGCAGCAACAACAACAACTACTGCGCCCTGGGCTGCTACGGCGGCT
GGCACCTGAGCGCCCTGCCCGGCAACACCCAGAGCAGCGGCCCCATCCCCGCCCTGGCCTGCGG
CCTGAGCTGCGACCACCAGGGCCTGGAGACCCAGCAGGGCGTGGCCTGGGTGCTGGCCGGCCAC
TGCCAGAGGCCCGGCCTGCACGAGGACCTGCAGGGCATGCTGCTGCCCAGCGTGCTGAGCAAGG
CCAGGAGCTGGACCTTCAGGGTGAAGTTCAGCAGGAGCGCCGACGCCCCCGCCTACAAGCAGGG
CCAGAACCAGCTGTACAACGAGCTGAACCTGGGCAGGAGGGAGGAGTACGACGTGCTGGACAAG
AGGAGGGGCAGGGACCCCGAGATGGGCGGCAAGCCCAGGAGGAAGAACCCCCAGGAGGGCCTGT
ACAACGAGCTGCAGAAGGACAAGATGGCCGAGGCCTACAGCGAGATCGGCATGAAGGGCGAGAG
GAGGAGGGGCAAGGGCCACGACGGCCTGTACCAGGGCCTGAGCACCGCCACCAAGGACACCTAC
GACGCCCTGCACATGCAGGCCCTGCCCCCCAGG
Murine anti-mMSLN scFv - mCD8 hinge - mCD28 TM and ICD - mIL9Ra ICD - mCD3z
(SEQ ID NO: 85)
MASPLTRFLSLNLLLLGESIILGSGEATRAQVQLQESGPGLVKPSQTLSLTCTVSGGSISSGGY
YWSWIRQHPGKGLEWIGYIYYSGSTYYNPSLKSRVTISVDTSKNQFSLKLSSVTAADTAVYYCA
RFDYGDFYDAFDIWGQGTMVTVSSGGGGSGGGGSGGGGSEIVLTQSPSSLSASVGDRVTITCRA
SQGISSALAWYQQKPGKAPKLLIYDASSLESGVPSRFSGSGSGTDFTLTISSLQPEDFATYYCQ
QFNSYPITFGQGTRLEIKRSGLQKVNSTTTKPVLRTPSPVHPTGTSQPQRPEDCRPRGSVKGTG
LDFACDIYFWALVVVAGVLFCYGLLVTVALCVIWTNSRRNRLLQSDYMNMTPRRPGLTRKPYQP
YAPARDFAAYRPKLSPRLKRIFYQNIPSPEAFFHPLYSVYHGDFQSWTGARRAGPQARQNGVST
SSAGSESSIWEAVATLTYSPACPVQFACLKWEATAPGFPGLPGSEHVLPAGCLELEGQPSAYLP
QEDWAPLGSARPPPPDSDSGSSDYCMLDCCEECHLSAFPGHTESPELTLAQPVALPVSSRAKFS
RSAETAANLQDPNQLYNELNLGRREEYDVLEKKRARDPEMGGKQQRRRNPQEGVYNALQKDKMA
EAYSEIGTKGERRRGKGHDGLYQGLSTATKDTYDALHMQTLAPR
Murine anti-mMSLN scFv - mCD8 hinge - mCD28 TM and ICD - mIL9Ra ICD - mCD3z
(SEQ ID NO: 86)
ATGGCTAGTCCGCTCACGAGGTTTTTGTCTCTGAACCTTCTCTTATTAGGGGAAAGCATAATCC
TGGGCAGCGGCGAGGCTACGCGGGCGCAGGTTCAGCTGCAAGAGTCCGGACCCGGTCTGGTGAA
GCCCAGCCAGACTTTGAGCCTGACCTGTACCGTATCTGGTGGCTCCATAAGTTCTGGAGGCTAC
TACTGGAGCTGGATAAGGCAGCACCCAGGGAAGGGCCTGGAGTGGATCGGCTATATTTACTACA
GCGGGAGCACTTATTATAATCCCTCATTAAAGAGCAGGGTCACCATCTCAGTGGACACATCCAA
GAACCAGTTCAGCTTGAAACTCTCTTCCGTAACAGCTGCTGACACTGCCGTTTACTATTGTGCC
AGGTTTGACTACGGAGATTTTTACGATGCCTTTGATATATGGGGCCAAGGCACCATGGTGACAG
TCTCCTCAGGTGGAGGAGGCAGTGGGGGGGGGGGGTCTGGGGGTGGTGGCTCTGAGATCGTTCT
AACCCAGAGCCCGAGCAGCCTATCGGCGTCAGTGGGAGATAGAGTGACCATTACCTGCAGGGCA
AGTCAAGGCATAAGCAGCGCTCTGGCCTGGTACCAACAAAAGCCTGGAAAGGCTCCTAAGCTGC
TGATTTATGATGCTTCGAGTCTCGAAAGTGGTGTCCCGTCAAGGTTTTCTGGTAGTGGTTCAGG
TACAGACTTCACCTTGACTATCAGCTCGCTCCAACCAGAAGATTTCGCAACATATTACTGCCAG
CAGTTCAACAGCTACCCCATTACATTTGGACAAGGAACCCGGCTTGAAATTAAACGCTCAGGGC
TTCAGAAGGTGAACAGCACAACAACCAAGCCAGTCTTGCGAACACCCAGTCCTGTTCACCCTAC
GGGTACGTCTCAACCTCAGAGGCCTGAGGACTGTAGACCCCGTGGCTCTGTGAAAGGGACAGGG
CTGGACTTTGCTTGTGACATCTACTTCTGGGCCCTGGTGGTGGTGGCCGGCGTGCTGTTCTGCT
ACGGCCTGCTGGTGACCGTGGCCCTGTGCGTGATCTGGACCAACAGCAGGAGGAACAGGCTGCT
GCAGAGCGACTACATGAACATGACCCCCAGGAGGCCCGGCCTGACCAGGAAGCCCTACCAGCCC
TACGCCCCCGCCAGGGACTTCGCCGCCTACAGGCCCAAACTATCTCCACGGCTCAAACGGATCT
TCTACCAGAACATCCCTTCTCCTGAAGCATTCTTCCATCCCCTGTATTCAGTTTATCATGGAGA
CTTCCAGTCCTGGACTGGGGCCCGCAGAGCTGGGCCACAAGCTCGACAGAATGGCGTGTCCACC
AGCTCTGCAGGGTCCGAGTCTTCCATTTGGGAGGCAGTGGCAACTCTGACTTACTCCCCAGCAT
GCCCTGTGCAGTTTGCCTGTCTGAAATGGGAAGCCACTGCCCCGGGCTTCCCAGGATTGCCGGG
CAGTGAGCATGTCCTGCCTGCAGGCTGCCTCGAACTCGAGGGCCAGCCATCTGCCTACCTGCCC
CAAGAAGACTGGGCCCCACTCGGCTCAGCTAGACCTCCCCCCCCAGATAGTGACTCCGGGTCGT
CTGACTATTGCATGCTGGATTGCTGTGAAGAGTGCCACCTGAGTGCCTTCCCTGGCCACACAGA
GAGTCCCGAGCTGACCTTGGCTCAGCCAGTAGCCCTCCCTGTCAGCTCCCGGGCCAAGTTTTCA
CGCTCTGCAGAGACAGCTGCCAACCTGCAGGACCCCAATCAGCTGTACAATGAACTGAATCTCG
GGCGGAGAGAAGAATATGATGTGTTGGAGAAGAAGCGTGCGAGAGACCCAGAGATGGGCGGCAA
ACAGCAGAGAAGACGAAACCCACAGGAAGGAGTGTACAACGCCCTGCAGAAAGACAAGATGGCA
GAGGCCTACTCAGAGATTGGAACCAAAGGAGAGAGGCGCCGTGGAAAAGGACATGATGGGCTTT
ACCAGGGTTTAAGTACGGCCACTAAAGATACTTATGACGCGCTGCACATGCAGACACTGGCACC
TCGA
Murine anti-mMSLN scFv - mCD8 hinge and TM - m41BB ICD - mIL9Ra ICD - mCD3z
(SEQ ID NO: 87)
MASPLTRFLSLNLLLLGESIILGSGEATRAQVQLQESGPGLVKPSQTLSLTCTVSGGSISSGGY
YWSWIRQHPGKGLEWIGYIYYSGSTYYNPSLKSRVTISVDTSKNQFSLKLSSVTAADTAVYYCA
RFDYGDFYDAFDIWGQGTMVTVSSGGGGSGGGGSGGGGSEIVLTQSPSSLSASVGDRVTITCRA
SQGISSALAWYQQKPGKAPKLLIYDASSLESGVPSRFSGSGSGTDFTLTISSLQPEDFATYYCQ
QFNSYPITFGQGTRLEIKRSGLQKVNSTTTKPVLRTPSPVHPTGTSQPQRPEDCRPRGSVKGTG
LDFACDIYIWAPLAGICVALLLSLIITLIKWIRKKFPHIFKQPFKKTTGAAQEEDACSCRCPQE
EEGGGGGYELKLSPRLKRIFYQNIPSPEAFFHPLYSVYHGDFQSWTGARRAGPQARQNGVSTSS
AGSESSIWEAVATLTYSPACPVQFACLKWEATAPGFPGLPGSEHVLPAGCLELEGQPSAYLPQE
DWAPLGSARPPPPDSDSGSSDYCMLDCCEECHLSAFPGHTESPELTLAQPVALPVSSRAKFSRS
AETAANLQDPNQLYNELNLGRREEYDVLEKKRARDPEMGGKQQRRRNPQEGVYNALQKDKMAEA
YSEIGTKGERRRGKGHDGLYQGLSTATKDTYDALHMQTLAPR
Murine anti-mMSLN scFv - mCD8 hinge and TM - m41BB ICD - mIL9Ra ICD - mCD3z
(SEQ ID NO: 88)
ATGGCTAGTCCGCTCACGAGGTTTTTGTCTCTGAACCTTCTCTTATTAGGGGAAAGCATAATCC
TGGGCAGCGGCGAGGCTACGCGGGCGCAGGTTCAGCTGCAAGAGTCCGGACCCGGTCTGGTGAA
GCCCAGCCAGACTTTGAGCCTGACCTGTACCGTATCTGGTGGCTCCATAAGTTCTGGAGGCTAC
TACTGGAGCTGGATAAGGCAGCACCCAGGGAAGGGCCTGGAGTGGATCGGCTATATTTACTACA
GCGGGAGCACTTATTATAATCCCTCATTAAAGAGCAGGGTCACCATCTCAGTGGACACATCCAA
GAACCAGTTCAGCTTGAAACTCTCTTCCGTAACAGCTGCTGACACTGCCGTTTACTATTGTGCC
AGGTTTGACTACGGAGATTTTTACGATGCCTTTGATATATGGGGCCAAGGCACCATGGTGACAG
TCTCCTCAGGTGGAGGAGGCAGTGGGGGGGGGGGGTCTGGGGGTGGTGGCTCTGAGATCGTTCT
AACCCAGAGCCCGAGCAGCCTATCGGCGTCAGTGGGAGATAGAGTGACCATTACCTGCAGGGCA
AGTCAAGGCATAAGCAGCGCTCTGGCCTGGTACCAACAAAAGCCTGGAAAGGCTCCTAAGCTGC
TGATTTATGATGCTTCGAGTCTCGAAAGTGGTGTCCCGTCAAGGTTTTCTGGTAGTGGTTCAGG
TACAGACTTCACCTTGACTATCAGCTCGCTCCAACCAGAAGATTTCGCAACATATTACTGCCAG
CAGTTCAACAGCTACCCCATTACATTTGGACAAGGAACCCGGCTTGAAATTAAACGCTCAGGGC
TTCAGAAGGTGAACAGCACAACAACCAAGCGAGTCTTGCGAACACCCAGTCCTGTTCACCCTAC
GGGTACGTCTCAACCTCAGAGGCCTGAGGACTGTAGACCCCGTGGCTCTGTGAAAGGGACAGGG
CTGGACTTTGCTTGTGACATCTACATCTGGGCACCCTTAGCCGGTATCTGTGTGGCCTTGCTGC
TTTCCCTCATCATCACTCTAATTAAGTGGATTCGAAAAAAGTTCCCCCACATCTTTAAGCAGCC
GTTCAAGAAAACCACTGGAGCAGCCCAGGAGGAGGATGCTTGCAGCTGCCGCTGTCCCCAGGAG
GAAGAAGGCGGCGGGGGCGGATATGAGCTCAAACTATCTCCACGGCTCAAACGGATCTTCTACC
AGAACATCCCTTCTCCTGAAGCATTCTTCCATCCCCTGTATTCAGTTTATCATGGAGACTTCCA
GTCCTGGACTGGGGCCCGCAGAGCTGGGCCACAAGCTCGACAGAATGGCGTGTCCACCAGCTCT
GCAGGGTCCGAGTCTTCCATTTGGGAGGCAGTGGCAACTCTGACTTACTCCCCAGCATGCCCTG
TGCAGTTTGCCTGTCTGAAATGGGAAGCCACTGCCCCGGGCTTCCCAGGATTGCCGGGCAGTGA
GCATGTCCTGCCTGCAGGCTGCCTCGAACTCGAGGGCCAGCCATCTGCCTACCTGCCCCAAGAA
GACTGGGCCCCACTCGGCTCAGCTAGACCTCCCCCCCCAGATAGTGACTCCGGGTCGTCTGACT
ATTGCATGCTGGATTGCTGTGAAGAGTGCCACCTGAGTGCCTTCCCTGGCCACACAGAGAGTCC
CGAGCTGACCTTGGCTCAGCCAGTAGCCCTCCCTGTCAGCTCCCGGGCCAAGTTTTCACGCTCT
GCAGAGACAGCTGCCAACCTGCAGGACCCCAATCAGCTGTACAATGAACTGAATCTCGGGCGGA
GAGAAGAATATGATGTGTTGGAGAAGAAGCGTGCGAGAGACCCAGAGATGGGCGGCAAACAGCA
GAGAAGACGAAACCCACAGGAAGGAGTGTACAACGCCCTGCAGAAAGACAAGATGGCAGAGGCC
TACTCAGAGATTGGAACCAAAGGAGAGAGGCGCCGTGGAAAAGGACATGATGGGCTTTACCAGG
GTTTAAGTACGGCCACTAAAGATACTTATGACGCGCTGCACATGCAGACACTGGCACCTCGA
Murine IL9Ra ICD
(SEQ ID NO: 124)
AAACTATCTCCACGGCTCAAACGGATCTTCTACCAGAACATCCCTTCTCCTGAAGCATTCTTCC
ATCCCCTGTATTCAGTTTATCATGGAGACTTCCAGTCCTGGACTGGGGCCCGCAGAGCTGGGCC
ACAAGCTCGACAGAATGGCGTGTCCACCAGCTCTGCAGGGTCCGAGTCTTCCATTTGGGAGGCA
GTGGCAACTCTGACTTACTCCCCAGCATGCCCTGTGCAGTTTGCCTGTCTGAAATGGGAAGCCA
CTGCCCCGGGCTTCCCAGGATTGCCGGGCAGTGAGCATGTCCTGCCTGCAGGCTGCCTCGAACT
CGAGGGCCAGCCATCTGCCTACCTGCCCCAAGAAGACTGGGCCCCACTCGGCTCAGCTAGACCT
CCCCCCCCAGATAGTGACTCCGGGTCGTCTGACTATTGCATGCTGGATTGCTGTGAAGAGTGCC
ACCTGAGTGCCTTCCCTGGCCACACAGAGAGTCCCGAGCTGACCTTGGCTCAGCCAGTAGCCCT
CCCTGTCAGCTCCCGGGCC
Anti-EGFR (806) scFv
(SEQ ID NO: 98)
DILMTQSPSSMSVSLGDTVSITCHSSQDINSNIGWLQQRPGKSFKGLIYHGTNLDDEVPSR
FSGSGSGADYSLTISSLESEDFADYYCVQYAQFPWTFGGGTKLEIKRGGGGSGGGGSGG
GGSDVQLQESGPSLVKPSQSLSLTCTVTGYSITSDFAWNWIRQFPGNKLEWMGYISYSG
NTRYNPSLKSRISITRDTSKNQFFLQLNSVTIEDTATYYCVTAGRGFPYWGQGTLVTVSA
Anti-EGFR (806) scFv
(SEQ ID NO: 99)
GATATTCTGATGACTCAATCTCCGTCTTCTATGAGCGTGAGCTTGGGTGACACCGTC
AGCATCACCTGTCATTCCAGCCAGGATATAAACTCAAATATCGGCTGGCTCCAGCAA
CGCCCAGGCAAGTCATTCAAGGGGCTTATTTATCATGGCACCAATCTTGACGATGAA
GTCCCATCACGCTTCAGCGGATCAGGCTCAGGTGCGGACTATTCCTTGACTATAAGT
TCCCTCGAATCTGAGGATTTCGCCGACTATTATTGCGTACAATACGCCCAGTTTCCCT
GGACCTTCGGAGGCGGCACCAAATTGGAGATAAAAAGGGGTGGAGGAGGATCAGG
CGGGGGTGGAAGCGGCGGAGGAGGCAGCGACGTACAACTGCAAGAATCCGGGCCG
AGTTTGGTCAAGCCCTCTCAATCTCTTTCTCTCACTTGCACGGTCACCGGATACTCCA
TAACCAGCGATTTTGCGTGGAATTGGATTCGACAATTTCCAGGGAATAAATTGGAAT
GGATGGGATATATCAGTTATTCTGGTAATACCAGATACAACCCGTCATTGAAAAGTC
GCATCTCTATAACACGAGACACTTCAAAGAATCAGTTCTTCCTTCAGCTCAATTCTGT
AACCATCGAAGATACTGCTACTTATTACTGTGTAACGGCGGGTCGAGGATTCCCCTA
CTGGGGCCAGGGTACACTGGTTACTGTTTCCGCC
Anti-IL13Ra2 (hu08) scFv
(SEQ ID NO: 100)
DIQMTQSPSSLSASVGDRVTITCKASQDVGTAVAWYQQIPGKAPKLLIYSASYRSTGVPD
RFSGSGSGTDFSFIISSLQPEDFATYYCQHHYSAPWTFGGGTKVEIKGGGGSGGGGSGGG
GSEVQLVESGGGLVQPGGSLRLSCAASGFTFSRNGMSWVRQTPDKRLEWVATVSSGGS
YIYYADSVKGRFTISRDNAKNSLYLQMSSLRAEDTAVYYCARQGTTALATRFFDVWGQ
GTLVTVSS
Anti-IL13Ra2 (hu08) scFv
(SEQ ID NO: 101)
GACATCCAAATGACTCAGAGCCCCTCTAGCCTCAGTGCAAGCGTCGGAGACCGGGT
GACCATCACCTGTAAAGCGTCCCAGGATGTTGGAACGGCAGTAGCTTGGTATCAAC
AAATCCCAGGGAAGGCTCCAAAGCTCCTTATATACTCTGCTAGTTACAGGTCCACCG
GGGTGCCCGACCGATTCTCTGGCTCCGGGAGCGGCACTGACTTTTCATTCATCATTA
GTAGTCTTCAACCTGAGGACTTTGCCACCTATTATTGCCAGCACCACTACTCTGCGC
CGTGGACTTTCGGAGGAGGCACGAAGGTTGAAATTAAAGGTGGAGGTGGGTCTGGC
GGAGGTGGAAGTGGTGGAGGCGGGTCCGAGGTTCAGTTGGTAGAGTCAGGCGGTGG
TCTGGTGCAGCCAGGTGGGTCCCTGCGCCTCAGCTGTGCAGCTTCCGGCTTTACTTTC
TCAAGGAATGGTATGTCCTGGGTACGGCAAACGCCGGACAAACGCCTTGAGTGGGT
AGCTACCGTATCCTCTGGGGGCTCTTACATATACTATGCAGACTCTGTGAAAGGAAG
ATTTACAATTTCACGCGACAATGCAAAAAATAGTTTGTACCTCCAAATGTCTAGTCT
TAGGGCCGAGGATACTGCCGTCTACTACTGTGCACGCCAGGGAACGACGGCTCTTG
CTACCCGATTTTTCGACGTTTGGGGCCAAGGAACGTTGGTGACAGTTAGCAG
Anti-hMSLN scFv M5 LCDR1
(SEQ ID NO: 102)
RASQSIRYYLS
Anti-hMSLN scFv M5 LCDR2
(SEQ ID NO: 103)
TASILQN
Anti-hMSLN scFv M5 LCDR3
(SEQ ID NO: 104)
LQTYTTPD
Anti-hMSLN scFv M5 HCDR1
(SEQ ID NO: 105)
GYTFTDYYMH
Anti-hMSLN scFv M5 HCDR2
(SEQ ID NO: 106)
WINPNSGGTNYAQKFQG
Anti-hMSLN scFv M5 HCDR3
(SEQ ID NO: 107)
GWDFDY
Anti-GD2 scFv
(SEQ ID NO: 108)
MALPVTALLLPLALLLHAARPGSDVVMTQTPLSLPVSLGDQASISCRSSQSLVHRNGNTYLHWY
LQKPGQSPKLLIHKVSNRFSGVPDRFSGSGSGTDFTLKISRVEAEDLGVYFCSQSTHVPPLTFG
AGTKLELKGGGGSGGGGSGGGGSGGGGSEVQLLQSGPELEKPSASVMISCKASGSSFTGYNMNW
VRQNIGKSLEWIGAIDPYYGGTSYNQKFKGRATLTVDKSSSTAYMHLKSLTSEDSVYYCVSGME
YWGQGTSVTVSSSG
Anti-GD2 scFv
(SEQ ID NO: 109)
ATGGCCTTACCAGTGACCGCCTTGCTCCTGCCGCTGGCCTTGCTGCTCCACGCCGCCAGGCCGG
GATCCGATGTTGTGATGACCCAAACTCCACTCTCCCTGCCTGTCAGTCTTGGAGATCAAGCCTC
CATCTCTTGCAGATCTAGTCAGAGTCTTGTAGACCGTAACGGAAACACCTATTTAGATTGGTAC
CTGCAGAAGCCAGGCCAGTCTCCAAAGCTCCTGATTCACAAAGTTTCCAACCGATTTTCTGGGG
TCCCAGACAGGTTCAGTGGCAGTGGATCAGGGACAGATTTCACACTCAAGATCAGCAGAGTGGA
GGCTGAGGATCTGGGAGTTTATTTCTGTTCTCAAAGTACACACGTTCCTCCGCTCACGTTCGGT
GCTGGGACCAAGCTGGAGCTGAAAGGAGGTGGCGGGTCAGGGGGTGGCGGAAGCGGAGGCGGCG
GTTCAGGCGGAGGAGGCTCGGAGGTGCAGCTTCTGCAGTCTGGACCTGAGCTGGAGAAGCCTTC
CGCTTCAGTGATGATATCCTGCAAGGCTTCTGGTTCCTCCTTCACTGGCTACAACATGAACTGG
GTGAGGCAGAATATTGGAAAGAGCCTTGAATGGATTGGAGCTATTGATCCTTACTACGGTGGAA
CTAGCTACAACCAGAAGTTCAAGGGCAGGGCCACATTGACTGTAGACAAATCGTCCAGCACAGC
CTACATGCACCTCAAGAGCCTGACATCTGAGGACTCTGTCTATTACTGTGTAAGCGGAATGGAG
TACTGGGGTCAAGGAACCTCAGTCACCGTCTCCTCATCCGGA
Anti-HER2 scFv (high affinity)
(SEQ ID NO: 110)
MDFQVQIFSFLLISASVIMSRGDIQMTQSPSSLSASVGDRVTITCRASQDVNTAVAWYQQKPGK
APKLLIYSASFLYSGVPSRFSGSRSGTDFTLTISSLQPEDFATYYCQQHYTTPPTFGQGTKVEI
KRTGSTSGSGKPGSGEGSEVQLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFYAMDVW
GQGTLVTVSS
Anti-HER2 scFv (high affinity)
(SEQ ID NO: 111)
ATGGATTTTCAGGTGCAGATTTTCAGCTTCCTGCTAATCAGTGCCTCAGTCATAATGTCCAGAG
GAGATATCCAGATGACCCAGTCCCCGAGCTCCCTGTCCGCCTCTGTGGGCGATAGGGTCACCAT
CACCTGCCGTGCCAGTCAGGATGTGAATACTGCTGTAGCCTGGTATCAACAGAAACCAGGAAAA
GCTCCGAAACTACTGATTTACTCGGCATCCTTCCTTTATTCTGGAGTCCCTTCTCGCTTCTCTG
GATCTAGATCTGGGACGGATTTCACTCTGACCATCAGCAGTCTGCAGCCGGAAGACTTCGCAAC
TTATTACTGTCAGCAACATTATACTACTCCTCCCACGTTCGGACAGGGTACCAAGGTGGAGATC
AAACGCACTGGGTCTACATCTGGATCTGGGAAGCCGGGTTCTGGTGAGGGTTCTGAGGTTCAGC
TGGTGGAGTCTGGCGGTGGCCTGGTGCAGCCAGGGGGCTCACTCCGTTTGTCCTGTGCAGCTTC
TGGCTTCAACATTAAAGACACCTATATACACTGGGTGCGTCAGGCCCCGGGTAAGGGCCTGGAA
TGGGTTGCAAGGATTTATCCTACGAATGGTTATACTAGATATGCCGATAGCGTCAAGGGCCGTT
TCACTATAAGCGCAGACACATCCAAAAACACAGCCTACCTGCAGATGAACAGCCTGCGTGCTGA
GGACACTGCCGTCTATTATTGTTCTAGATGGGGAGGGGACGGCTTCTATGCTATGGACGTGTGG
GGTCAAGGAACCCTGGTCACCGTCTCCTCG
Anti-HER2 scFv (low affinity)
(SEQ ID NO: 112)
MDFQVQIFSFLLISASVIMSRGDIQMTQSPSSLSASVGDRVTITCRASQDVNTAVAWYQQKPGK
APKLLIYSASFLESGVPSRFSGSRSGTDFTLTISSLQPEDFATYYCQQHYTTPPTFGQGTKVEI
KRTGSTSGSGKPGSGEGSEVQLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFVAMDVW
GQGTLVTVSS
Anti-HER2 scFv (low affinity)
(SEQ ID NO: 113)
ATGGATTTTCAGGTGCAGATTTTCAGCTTCCTGCTAATCAGTGCCTCAGTCATAATGTCCAGAG
GAGATATCCAGATGACCCAGTCCCCGAGCTCCCTGTCCGCCTCTGTGGGCGATAGGGTCACCAT
CACCTGCCGTGCCAGTCAGGATGTGAATACTGCTGTAGCCTGGTATCAACAGAAACCAGGAAAA
GCTCCGAAACTACTGATTTACTCGGCATCCTTCCTTGAGTCTGGAGTCCCTTCTCGCTTCTCTG
GATCTAGATCTGGGACGGATTTCACTCTGACCATCAGCAGTCTGCAGCCGGAAGACTTCGCAAC
TTATTACTGTCAGCAACATTATACTACTCCTCCCACGTTCGGACAGGGTACCAAGGTGGAGATC
AAACGCACTGGGTCTACATCTGGATCTGGGAAGCCGGGTTCTGGTGAGGGTTCTGAGGTTCAGC
TGGTGGAGTCTGGCGGTGGCCTGGTGCAGCCAGGGGGCTCACTCCGTTTGTCCTGTGCAGCTTC
TGGCTTCAACATTAAAGACACCTATATACACTGGGTGCGTCAGGCCCCGGGTAAGGGCCTGGAA
TGGGTTGCAAGGATTTATCCTACGAATGGTTATACTAGATATGCCGATAGCGTCAAGGGCCGTT
TCACTATAAGCGCAGACACATCCAAAAACACAGCCTACCTGCAGATGAACAGCCTGCGTGCTGA
GGACACTGCCGTCTATTATTGTTCTAGATGGGGAGGGGACGGCTTCGTTGCTATGGACGTGTGG
GGTCAAGGAACCCTGGTCACCGTCTCCTCG
Anti-TnMuc1 scFv
(SEQ ID NO: 114)
QVQLQQSDAELVKPGSSVKISCKASGYTFTDHAIHWVKQKPEQGLEWIGHFSPGNTDIKYNDKF
KGKATLTVDRSSSTAYMQLNSLTSEDSAVYFCKTSTFFFDYWGQGTTLTVSSGGGGSGGGGSGG
GGSELVMTQSPSSLTVTAGEKVTMICKSSQSLLNSGDQKNYLTWYQQKPGQPPKLLIFWASTRE
SGVPDRFTGSGSGTDFTLTISSVQAEDLAVYYCQNDYSYPLTFGAGTKLELK
Anti-TnMuc1 scFv
(SEQ ID NO: 115)
CAGGTGCAGCTGCAGCAGTCTGATGCCGAGCTCGTGAAGCCTGGCAGCAGCGTGAAGATCAGCT
GCAAGGCCAGCGGCTACACCTTCACCGACCACGCCATCCACTGGGTCAAGCAGAAGCCTGAGCA
GGGCCTGGAGTGGATCGGCCACTTCAGCCCCGGCAACACCGACATCAAGTACAACGACAAGTTC
AAGGGCAAGGCCACCCTGACCGTGGACAGAAGCAGCAGCACCGCCTACATGCAGCTGAACAGCC
TGACCAGCGAGGACAGCGCCGTGTACTTCTGCAAGACCAGCACCTTCTTTTTCGACTACTGGGG
CCAGGGCACAACCCTGACAGTGTCTAGCGGAGGCGGAGGATCTGGCGGCGGAGGAAGTGGCGGA
GGGGGATCTGAACTCGTGATGACCCAGAGCCCCAGCTCTCTGACAGTGACAGCCGGCGAGAAAG
TGACCATGATCTGCAAGTCCTCCCAGAGCCTGCTGAACTCCGGCGACCAGAAGAACTACCTGAC
CTGGTATCAGCAGAAACCCGGCCAGCCCCCCAAGCTGCTGATCTTTTGGGCCAGCACCCGGGAA
AGCGGCGTGCCCGATAGATTCACAGGCAGCGGCTCCGGCACCGACTTTACCCTGACCATCAGCT
CCGTGCAGGCCGAGGACCTGGCCGTGTATTACTGCCAGAACGACTACAGCTACCCCCTGACCTT
CGGAGCCGGCACCAAGCTGGAACTGAAG
Anti-CD70 scFv
(SEQ ID NO: 116)
MALPVTALLLPLALLLHAARPQAVVTQEPSLTVSPGGTVTLTCGLKSGSVTSDNFPTWYQQTPG
QAPRLLIYNTNTRHSGVPDRFSGSILGNKAALTITGAQADDEAEYFCALFISNPSVEFGGGTQL
TVLGGGGGSGGGGSGGGGSGGGGSEVQLVESGGGLVQPGGSLRLSCAASGFTFSVYYMNWVRQA
PGKGLEWVSDINNEGGTTYYADSVKGRFTISRDNSKNSLYLQMNSLRAEDTAVYYCARDAGYSN
HVPIFDSWGQGTLVTVSS
Anti-CD70 scFv
(SEQ ID NO: 117)
ATGGCCTTACCAGTGACCGCCTTGCTCCTGCCGCTGGCCTTGCTGCTCCACGCCGCCAGGCCGC
AGGCCGTTGTCACACAAGAGCCCTCTCTGACCGTGTCACCCGGGGGGACCGTGACCCTGACATG
TGGTTTGAAATCTGGAAGTGTAACATCCGACAACTTTCCTACATGGTACCAGCAGACGCCTGGT
CAAGCGCCGCGATTGCTGATTTATAATACCAATACACGCCACAGCGGAGTACCTGACAGATTCA
GTGGCAGCATCCTTGGAAACAAAGCGGCACTGACCATAACAGGTGCCCAAGCAGATGATGAAGC
AGAGTACTTCTGTGCCCTCTTTATTAGTAATCCCTCAGTTGAATTTGGGGGTGGTACACAACTT
ACAGTTCTCGGTGGTGGCGGAGGATCAGGGGGGGGAGGAAGTGGTGGTGGCGGCAGTGGCGGAG
GTGGGAGTGAGGTTCAGCTCGTAGAATCAGGAGGAGGTTTGGTACAACCGGGCGGCTCTCTGAG
ACTTTCATGCGCTGCGAGCGGGTTTACTTTCTCTGTCTATTATATGAATTGGGTGAGACAGGCG
CCGGGAAAGGGGCTGGAATGGGTGAGTGATATTAACAATGAAGGAGGTACCACCTACTACGCGG
ACAGTGTAAAGGGCAGATTTACCATAAGCCGGGATAACAGTAAGAACAGTCTTTACTTGCAAAT
GAATTCACTGCGAGCGGAGGATACCGCGGTATACTACTGTGCTAGGGACGCGGGTTACAGTAAC
CATGTGCCAATTTTCGATTCTTGGGGACAGGGAACCCTCGTCACCGTGTCCAGC
Anti-CD70 tr27 CAR (tr27-h41BB-hCD3zeta)
(SEQ ID NO: 118)
ATGGCTCGGCCCCATCCCTGGTGGTTGTGTGTGCTGGGAACACTTGTCGGCCTGAGTGCTACCC
CTGCCCCTAAATCATGCCCGGAACGGCACTATTGGGCGCAGGGTAAACTGTGCTGCCAAATGTG
TGAACCAGGTACTTTTCTGGTCAAAGATTGCGATCAACACAGGAAGGCAGCTCAATGCGATCCT
TGTATCCCTGGGGTGAGCTTCAGCCCCGACCATCATACTAGACCACATTGTGAAAGTTGCCGAC
ACTGTAATAGCGGACTCTTGGTCCGCAATTGCACCATTACCGCTAATGCCGAATGCGCCTGCCG
CAATGGATGGCAGTGCCGGGACAAGGAGTGCACAGAGTGCGACCCTCTCCCAAATCCAAGTCTC
ACGGCTCGGTCCAGTCAGGCGCTTAGCCCGCACCCACAACCTACTCACCTGCCCTACGTCTCTG
AAATGTTGGAAGCGAGAACAGCAGGTCACATGCAAACACTTGCGGACTTTCGGCAGCTGCCTGC
GCGCACACTTTCAACCCATTGGCCACCACAACGGAGTCTGTGTAGTTCCGACTTCATAAGAATC
CTCGTTATCTTCTCTGGGATGTTCTTGGTATTCACGTTGGCCGGCGCCCTGTTTCTCCGGTTCA
GTGTAGTGAAACGGGGCAGAAAGAAACTCCTGTATATATTCAAACAACCATTTATGAGACGAGT
ACAAACTACTCAAGAGGAAGATGGCTGTAGCTGCCGATTTCCAGAAGAAGAAGAAGGAGGATGT
GAACTGAGAGTGAAGTTCAGCAGGAGCGCAGACGCCCCCGCGTACCAGCAGGGCCAGAACCAGC
TCTATAACGAGCTCAATCTAGGACGAAGAGAGGAGTACGATGTTTTGGACAAGAGACGTGGCCG
GGACCCTGAGATGGGGGGAAAGCCGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGGCA
AGGGGCACGATGGCCTTTACCAGGGTCTCAGTACAGCCACCAAGGACACCTACGACGCCCTTCA
CATGCAGGCCCTGCCCCCTCGCTAA
Anti-CD70 tr27 CAR (tr27-h41BB-hCD3zeta)
(SEQ ID NO: 119)
MARPHPWWLCVLGTLVGLSATPAPKSCPERHYWAQGKLCCQMCEPGTFLVKDCDQHRKAAQCDP
CIPGVSFSPDHHTRPHCESCRHCNSGLLVRNCTITANAECACRNGWQCRDKECTECDPLPNPSL
TARSSQALSPHPQPTHLPYVSEMLEARTAGHMQTLADFRQLPARTLSTHWPPQRSLCSSDFIRI
LVIFSGMFLVFTLAGALFLRFSVVKRGRKKLLYIFKQPFMRPVQTTQEEDGCSCRFPEEEEGGC
ELRVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
Anti-PMSA scFv
(SEQ ID NO: 120)
MALPVTALLLPLALLLHAARPGSDIVMTQSHKFMSTSVGDRVSIICKASQDVGTAVDWYQQKPG
QSPKLLIYWASTRHTGVPDRFTGSGSGTDFTLTITNVQSEDLADYFCQQYNSYPLTFGAGTMLD
LKGGGGSGGGGSSGGGSEVQLQQSGPELVKPGTSVRISCKTSGYTFTEYTIHWVKQSHGKSLEW
IGNINPNNGGTTYNQKFEDKATLTVDKSSSTAYMELRSLTSEDSAVYYCAAGWNFDYWGQGTTL
TVSSASSG
Anti-PMSA scFv
(SEQ ID NO: 121)
ATGGCCCTGCCTGTGACAGCCCTGCTGCTGCCTCTGGCTCTGCTGCTGCACGCCGCCAGACCTG
GATCTGAGATTGTGATGAGCGAGTCTCACAAATTCATGTCCACATGAGTAGGAGACAGGGTGAG
CATCATCTGTAAGGCCAGTCAAGATGTGGGTACTGCTGTAGACTGGTATCAACAGAAACCAGGA
CAATCTCCTAAACTACTGATTTATTGGGCATCCACTCGGCACACTGGAGTCCCTGATCGCTTCA
CAGGCAGTGGATCTGGGACAGACTTCACTCTCACCATTACTAACGTTCAGTCTGAAGACTTGGC
AGATTATTTCTGTCAGCAATATAACAGCTATCCTCTCACGTTCGGTGCTGGGACCATGCTGGAC
CTGAAAGGAGGCGGAGGATCTGGCGGCGGAGGAAGTTCTGGCGGAGGCAGCGAGGTGCAGCTGC
AGCAGAGCGGACCCGAGCTCGTGAAGCCTGGAACAAGCGTGCGGATCAGCTGCAAGACCAGCGG
CTACACCTTCACCGAGTACACCATCCACTGGGTCAAGCAGTCCCACGGCAAGAGCCTGGAGTGG
ATCGGCAATATCAACCCCAACAACGGCGGCACCACCTACAACCAGAAGTTCGAGGACAAGGCCA
CCCTGACCGTGGACAAGAGCAGCAGCACCGCCTACATGGAACTGCGGAGCCTGACCAGCGAGGA
CAGCGCCGTGTACTATTGTGCCGCCGGTTGGAACTTCGACTACTGGGGCCAGGGCACAACCCTG
ACAGTGTCTAGCGCTAGCTCCGGA
Anti-EGFRvIII scFv
(SEQ ID NO: 122)
MALPVTALLLPLALLLHAARPEIQLVQSGAEVKKPGESLRISCKGSGFNIEDYYIHWVRQMPGK
GLEWMGRIDPENDETKYGPIFQGHVTISADTSINTVYLQWSSLKASDTAMYYCAFRGGVYWGQG
TTVTVSSGGGGSGGGGSGGGGSGGGGSDVVMTQSPDSLAVSLGERATINCKSSQSLLDSDGKTY
LNWLQQKPGQPPKRLISLVSKLDSGVPDRFSGSGSGTDFTLTISSLQAEDVAVYYCWQGTHFPG
TFGGGTKVEIK
Anti-EGFRvIII scFv
(SEQ ID NO: 123)
ATGGCCCTCCCTGTCACCGCCCTGCTGCTTCCGCTGGCTCTTCTGCTCCACGCCGCTCGGCCCG
AGATTCAGCTCGTGCAATCGGGAGCGGAAGTCAAGAAGCCAGGAGAGTCCTTGCGGATCTCATG
CAAGGGTAGCGGCTTTAACATCGAGGATTACTACATCCACTGGGTGAGGCAGATGCCGGGGAAG
GGACTCGAATGGATGGGACGGATCGACCCAGAAAACGACGAAACTAAGTACGGTCCGATCTTCC
AAGGCCATGTGACTATTAGCGCCGATACTTCAATCAATACCGTGTATCTGCAATGGTCCTCATT
GAAAGCCTCAGATACCGCGATGTACTACTGTGCTTTCAGAGGAGGGGTCTACTGGGGACAGGGA
ACTACCGTGACTGTCTCGTCCGGCGGAGGCGGGTCAGGAGGTGGCGGCAGCGGAGGAGGAGGGT
CCGGCGGAGGTGGGTCCGACGTCGTGATGACCCAGAGCCCTGACAGCCTGGCAGTGAGCCTGGG
CGAAAGAGCTACCATTAACTGCAAATCGTCGCAGAGCCTGCTGGACTCGGACGGAAAAACGTAC
CTCAATTGGCTGCAGCAAAAGCCTGGCCAGCCACCGAAGCGCCTTATCTCACTGGTGTCGAAGC
TGGATTCGGGAGTGCCCGATCGCTTCTCCGGCTCGGGATCGGGTACTGACTTCACCCTCACTAT
CTCCTCGCTTCAAGCAGAGGACGTGGCCGTCTACTACTGCTGGCAGGGAACCCACTTTCCGGGA
ACCTTCGGCGGAGGGACGAAAGTGGAGATCAAG

### C. Nucleic Acids and Expression Vectors

In one aspect, the invention provides an isolated nucleic acid comprising a nucleotide sequence encoding a chimeric antigen receptor (CAR) comprising a tumor antigen binding domain, a transmembrane domain, and an intracellular domain comprising an intracellular signaling domain of an interleukin-9 receptor alpha (IL9Ra).

In certain embodiments, a nucleic acid of the present disclosure comprises a first nucleotide sequence and a second nucleotide sequence. The first and second nucleotide sequences may be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. In certain embodiments, the nucleic acid comprises from 5′ to 3′ the first nucleotide sequence, the linker, and the second nucleotide sequence. In certain embodiments, the nucleic acid comprises from 5′ to 3′ the second nucleotide sequence, the linker, and the first nucleotide sequence.

In some embodiments, the linker comprises a nucleic acid sequence that encodes an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention.

In some embodiments, the linker comprises a nucleic acid sequence that encodes a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAV0, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use in the present invention.

In some embodiments, the construct includes a linker that optionally, further comprises a nucleic acid sequence that encodes a furin cleavage site. Furin is a ubiquitously expressed protease that resides in the trans-golgi and processes protein precursors before their secretion. Furin cleaves at the COOH-terminus of its consensus recognition sequence. Various furin consensus recognition sequences (or “furin cleavage sites”) are known to those of skill in the art. Those of skill in the art would be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding a Furin cleavage site and F2A, a linker comprising a nucleic acid sequence encoding a Furin cleavage site and E2A, a linker comprising a nucleic acid sequence encoding a Furin cleavage site and P2A, a linker comprising a nucleic acid sequence encoding a Furin cleavage site and T2A. Those of skill in the art would be able to select the appropriate combination for use in the present invention. In such embodiments, the linker may further comprise a spacer sequence between the Furin cleavage site and the 2A peptide. In some embodiments, the linker comprises a Furin cleavage site 5′ to a 2A peptide. In some embodiments, the linker comprises a 2A peptide 5′ to a Furin cleavage site. Various spacer sequences are known in the art, including, without limitation, glycine serine (GS) spacers (also known as GS linkers). Those of skill in the art would be able to select the appropriate spacer sequence for use in the present invention.

In some embodiments, a nucleotide sequence of the present disclosure may be operably linked to a transcriptional control element, e.g., a promoter, and enhancer, etc. Suitable promoter and enhancer elements are known to those of skill in the art.

In certain embodiments, the promoter is selected from a phosphoglycerate kinase-1 (PGK) promoter, an EF-1a promoter, and a CMV promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is used for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the nucleic acid into an immune cell or precursor thereof (e.g., a T cell). Accordingly, an expression vector (e.g., a lentiviral vector) of the present invention may comprise a nucleic acid of the invention comprising one or more nucleotide sequences encoding a CAR of the invention. In some embodiments, the expression vector (e.g., lentiviral vector) will comprise additional elements that will aid in the functional expression of the receptors encoded therein.

In some embodiments, an expression vector comprising a nucleic acid of the invention further comprises an elongation-factor-1-alpha promoter (EF-1α promoter). Use of an EF-1α promoter may increase the efficiency in expression of downstream transgenes (e.g., a CAR encoding nucleotide sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding a CAR of the invention.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15: 9053. Introducing RNA comprising a nucleotide sequence encoding a CAR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of a CAR as described herein, e.g., in a mammalian cell. In some embodiments, a nucleic acid of the present disclosure provides for amplification of the nucleic acid.

### D. Modified Immune Cells

The present invention additionally provides a modified cell, wherein the cell is an immune cell or precursor cell thereof, and wherein the cell is engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

In certain embodiments, the modified immune cell or precursor cell thereof is selected from a T cell, a natural killer T (NKT) cell, a gamma-delta T cell, a natural killer (NK) cell, and a macrophage.

In certain embodiments, the modified cell is an autologous cell. In certain embodiments, the modified cell is an autologous cell. In some embodiments, the cell is a human cell obtained from a human subject. In certain embodiments, the modified cell is a T cell.

### E. Methods of Treatment

In another aspect, the invention provides a method of treating cancer in a subject in need thereof, comprising administering to the subject a population of modified cells, wherein the cells are immune cells or precursor cells thereof, and wherein the cells are engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

The modified cell (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a population of modified cells of the present invention, wherein the cells are immune cells or precursor cells thereof (e.g., T cells).

Methods for administration of immune cells for adoptive cell therapy are known and may be used in connection with the provided methods and compositions. For example, adoptive immune cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338; Lee et al., Int J. Mol Sci. (2021) 22(9):4590; Banerjee et al., JCO Clin Cancer Inform. (2021) 5:668-678; Robbins et al., Stem Cell Res Ther. (2021) 12(1):350; Wrona et al., Int J Mol Sci. (2021) 22(11):5899; Atrash and Moyo, Onco Targets Ther. (2021) 14:2185-2201; Martinez Bedoya et al., Front Immunol. (2021) 12:640082; Morgan et al., Front Immunol. (2020) 11:1965; Chicaybam et al., Cancers (Basel) (2020) 12(9):2360; and Rafiq et al., Nat Rev Clin Oncol. (2020) 17(3):147-167. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cell of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include certain leukemia or lymphoid malignancies, benign and malignant tumors, and malignancies e.g., sarcomas, carcinomas, and melanomas. Exemplary cancers include but are not limited to B-cell malignancies (such as B-cell lymphomas and leukemias and the like), lung cancer, non-small cell lung cancer, small cell lung cancer, Merkel cell carcinoma, melanoma, Hodgkin's lymphoma, non-Hodgkin's lymphoma, urothelial carcinoma, gastric cancer, cervical cancer, cutaneous squamous cell carcinoma, renal cell carcinoma, breast cancer, triple-negative breast cancer, colon cancer, esophagus cancer, stomach cancer, liver cancer, kidney cancer, pancreatic cancer, prostate cancer, brain cancer, lung adenocarcinoma, glioblastoma, hepatocellular carcinoma, gallbladder cancer, cervical cancer, cervical squamous cell carcinoma, colorectal cancer, ovarian cancer, and renal cancer. The cancers may be non-solid tumors (such as hematological tumors) or solid tumors. Adult tumors/cancers and pediatric tumors/cancers are also included. In one embodiment, the cancer is a solid tumor or a hematological tumor.

The cells to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, alymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10′ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present invention.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009); Herman et al. J. Immunological Methods, 285(1): 25-40 (2004); Kiesgen et al., Nat Protoc. (2021) 16(3):1331-1342; and Maldini et al., J Immunol Methods (2020) 484-485:112830. In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In one aspect, the invention includes a method of treating cancer in a subject in need thereof, comprising administering to the subject any one of the modified immune or precursor cells disclosed herein. Yet another aspect of the invention includes a method of treating cancer in a subject in need thereof, comprising administering to the subject a modified immune or precursor cell generated by any one of the methods disclosed herein.

### F. Sources of Immune Cells

In certain embodiments, a source of immune cells (e.g. T cells) is obtained from a subject for ex vivo manipulation and/or in vivo transduction. Sources of target cells for ex vivo manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human. Methods for in vivo transduction of immune cells for CAR expression are described, e.g., in Pfeiffer et al., EMBO Mol Med. (2018) 10(11):e9158; Weidner et al., Nat Protoc. (2021) 16(7):3210-3240; Frank et al., Blood Advances (2020) 4(22):5702-5715; Nawaz et al., Blood Cancer J. (2021) 11(6):119.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a macrophage, or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8⁺ naive T cell, central memory T cell, or effector memory T cell), a CD4⁺ T cell, a stem cell memory T cell, a lymphoid progenitor cell, or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4⁺ and/or of CD8⁺ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8⁺ cells or the T cells, e.g., CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8⁺ T cells are enriched for cells positive for CD45RO (or negative for CD45RA) and for CD62L. For example, CD3⁺, CD28⁺ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8⁺ T cells and CD4⁺ T cells further enhances efficacy.

In some embodiments, memory T cells are present in both CD62L⁺ and CD62L− subsets of CD8⁺ peripheral blood lymphocytes. PBMC can be enriched for or depleted of CD62L−CD8+ and/or CD62L⁺CD8⁺ fractions, such as using anti-CD8 and anti-CD62L antibodies. In some embodiments, a CD4⁺ T cell population and a CD8⁺ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

CD4⁺ T helper cells are sorted into naive, central memory, and effector cells by identifying cell populations that have cell surface antigens. CD4⁺ lymphocytes can be obtained by standard methods. In some embodiments, naive CD4⁺ T lymphocytes are CD45RO−, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L− and CD45RO. In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. n yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### G. Expansion of Immune Cells

Whether prior to or after modification of cells to express a CAR of the invention, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### H. Pharmaceutical Compositions and Formulations

Also provided are populations of immune cells of the invention, compositions containing such cells and/or enriched for such cells, such as in which cells expressing a CAR (e.g., a CAR comprising an IL-make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Retroviruses encoding chimeric antigen receptors were produced utilizing Plat-E packaging cells (Cell Biolabs) by transfection with pMSGV vectors using Lipofectamine 2000 (ThermoFisher). Culture media was replaced 24 h later and after an additional 24 h the media was collected, clarified by centrifugation, and passed through a 0.45 um filter before storage at −80 degrees celsius.

Retroviral transduction of mouse CAR T cells was achieved by enriching donor CD45.1+ mouse bulk splenocytes for CD3+ cells by magnetic bead separation (Stemcell Technologies). T cells were activated with mouse CD3/CD28 Dynabeads (ThermoFisher) in the presence of 50 U/ml recombinant human IL-2 (Peprotech) for 48 h before spinfection (1 h) with retroviral supernatant on retronectin-coated (Takara Bio) plates. Cells were harvested for flow cytometry two days after spinfection.

Flow cytometric detection of chimeric antigen receptors was performed by incubating retrovirally transduced mouse T cells with antibodies specific for CAR for 20 minutes in room temperature in the dark followed by acquisition of at least 10,000 events on LSR II flow cytometer (BD Biosciences). Data were analyzed with FlowJo software (BD Biosciences).

The results of the experiments are now described.

### Example 1: Chimeric Antigen Receptors Comprising IL9Ra ICD

Adoptively transferred gene engineered T cell therapies have demonstrated significant antitumor activity in patients with hematopoietic malignancies, but have limited benefit in patients with solid tumors (Rosenberg and Restifo, Science (2015) 348:62-68). One major limitation is the poor in vivo expansion and persistence of adoptively transferred T cells, necessitating lymphodepleting conditioning chemotherapy—a toxic regimen that limits patient eligibility (Goff et al., J Clin Oncol. (2016) 34:2389-2397; Dudley et al., J Clin Oncol. (2008) 26:5233-5239; Dutcher et al., Journal for ImmunoTherapy of Cancer (2014) 2:26). Even those T cells that do expand and persist become terminally differentiated and dysfunctional (Philip et al., Nature (2017) 545:452-456; Schietinger et al., Immunity (2016) 45:389-401). T cells with a stem-like phenotype can overcome these limitations and exhibit superior antitumor activity in mouse models and humans (Gattinoni et al., Nature Reviews Cancer (2012) 12:671-684; Krishna et al., Science (2020) 370:1328-1334), but therapeutic manipulations to select or expand stem-like T cells are limited to the cell manufacturing phase and cannot be made in vivo.

The IL-9 receptor (CD129) is a less-studied member of γc cytokine receptor family and binds to IL-9, forming a heteroderimic receptor signaling complex with γc that can result in pSTAT1, pSTAT3 and pSTAT5 activation (Demoulin et al., Mol Cell Biol. (1996) 16:4710-4716; Knoops et al., Growth Factors (2004) 22:207-215; Bauer et al., J Biol Chem. (1998) 273:9255-9260). IL-9R is naturally expressed by mast cells, memory B cells, innate lymphoid cells, and hematopoietic progenitors (Knoops et al., Growth Factors (2004) 22:207-215; Takatsuka et al., Nature Immunology (2018) 19:1025-1034; Townsend et al., Immunity (2000) 13:573-583; Williams et al., Blood (1990) 76:906-911; Turner et al., J Exp Med (2013) 210:2951-2965). While T cell subsets that produce TL-9 have been described (Lu et al., J Clin Invest (2012) 122:4160-4171; Lu et al., Proc Natl Acad Sci USA (2014) 111:2265; Purwar et al., Nat Med (2012) 18:1248-1253), the effects of IL-9R signaling on T cells are not well characterized (Elyaman et al., Proc Natl Acad Sci USA (2009) 106:12885-12890; Nowak et al., J Exp Med (2009) 206:1653-1660; Li et al., Eur J Immunol (2011) 41:2197-2206; Houssiau et al., J Immunol (1993) 150:2634-2640; Louahed et al., J Immunol (1995) 154:5061-5070; Lehrnbecher et al., Cytokine (1994) 6:279-284). For example, it has been well-documented that naïve T cells are insensitive to IL-9 and T cell development is unimpaired in IL-9 deficient mice suggesting that IL-9 is not be a critical natural cytokine in T cell biology (Townsend et al., Immunity (2000) 13:573-583; Houssiau et al., J Immunol (1993) 150:2634-2640). More recently, common gamma chain (γc) cytokine receptor signaling with STAT1, STAT3 and STAT5 activation was observed in T cells engineered to express orthogonal chimeric cytokine receptors (WO 2021/050752; Kalbasi, et al., 2022, Nature, 607(7918):360-365). These T cells assume characteristics of stem cell memory and effector T cells.

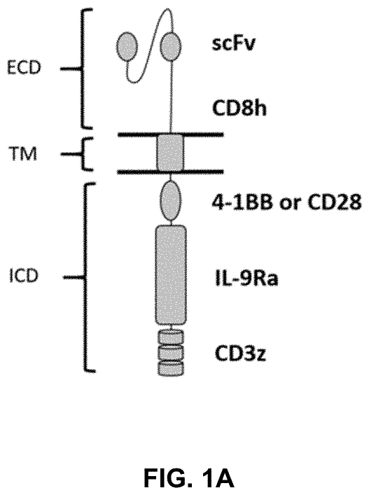

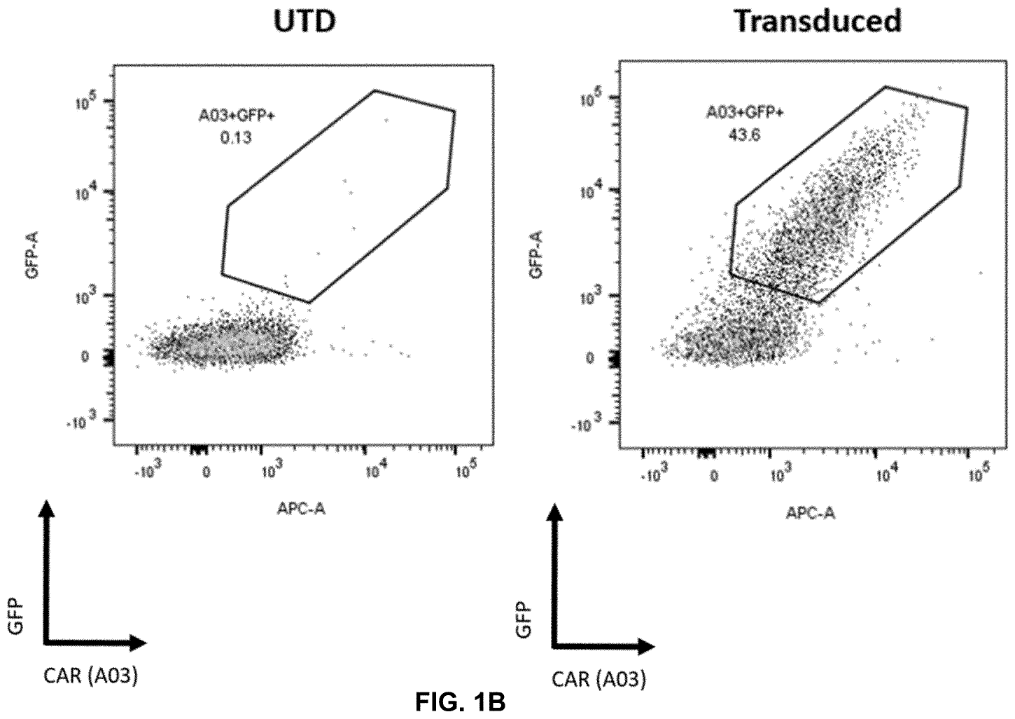

As an alternative approach to convey T cells with common gamma chain (γc) cytokine receptor signaling, a chimeric antigen receptor (CAR) comprising an IL9Ra ICD was designed (FIG. 1A). Human and murine versions of this CAR were designed as outlined in Table 1.

TABLE 1
Chimeric Antigen Receptors Comprising IL9Ra ICD

| CAR | scFv | Hinge and TM | ICD | SEQ ID NOs |
|---|---|---|---|---|
| Human | anti- | hCD8 hinge - | hCD28 - | SEQ ID NO: 81 - |
| IL9Ra | hMSLN | hCD28 TM | hIL9Ra - | amino acid |
| | | | hCD3z | SEQ ID NO: 82 - |
| | | | | nucleotide |
| Human - | anti- | hCD8 hinge | h41BB - | SEQ ID NO: 83 - |
| IL9Ra | hMSLN | and TM | hIL9Ra - | amino acid |
| | | | hCD3z | SEQ ID NO: 84 - |
| | | | | nucleotide |
| Murine - | anti- | mCD8 hinge - | mCD28 - | SEQ ID NO: 85 - |
| IL9Ra | mMSLN | mCD28 TM | mIL9Ra - | amino acid |
| | | | mCD3z | SEQ ID NO: 86 - |
| | | | | nucleotide |
| Murine - | anti- | mCD8 hinge | m41BB - | SEQ ID NO: 87 - |
| IL9Ra | mMSLN | and TM | mIL9Ra - | amino acid |
| | | | mCD3z | SEQ ID NO: 88 - |
| | | | | nucleotide |

Expression of the murine CAR (comprising an anti-MSLN scFv, CD8 hinge and TM, 41BB ICD, IL9Ra ICD, and CD3z stimulatory domain) from a lentiviral construct was tested and demonstrated on transduced murine T cells (FIG. 1B). A significant advantage of the CARs of the invention compared to orthogonal cytokine receptor systems is that they do not require administration of an orthogonal cytokine.

It is expected that expression of these CARs comprising an IL9Ra ICD on transduced T cells will activate STAT1, STAT3 and STAT5 in the T cells, and thereby convey the T cells with stem cell memory (Tscm) features with improved trafficking and effector function, resulting in improved antitumor activity for hard-to-treat solid tumors in patients.

### Example 2: Co-Expression of WT IL9Ra and a CAR in T Cells

It is contemplated herein that co-expressing an IL9Ra receptor, or a chimeric cytokine receptor comprising an IL9Ra ICD, together with a CAR targeting a tumor antigen on transduced T cells, will similarly activate STAT1, STAT3 and STAT5 in the T cells in vivo, and thereby convey the T cells with stem cell memory (Tscm) features with improved trafficking and effector function, resulting in improved antitumor activity for hard-to-treat solid tumors in patients (as was previously shown using orthogonal chimeric cytokine receptors comprising an IL9Ra ICD).

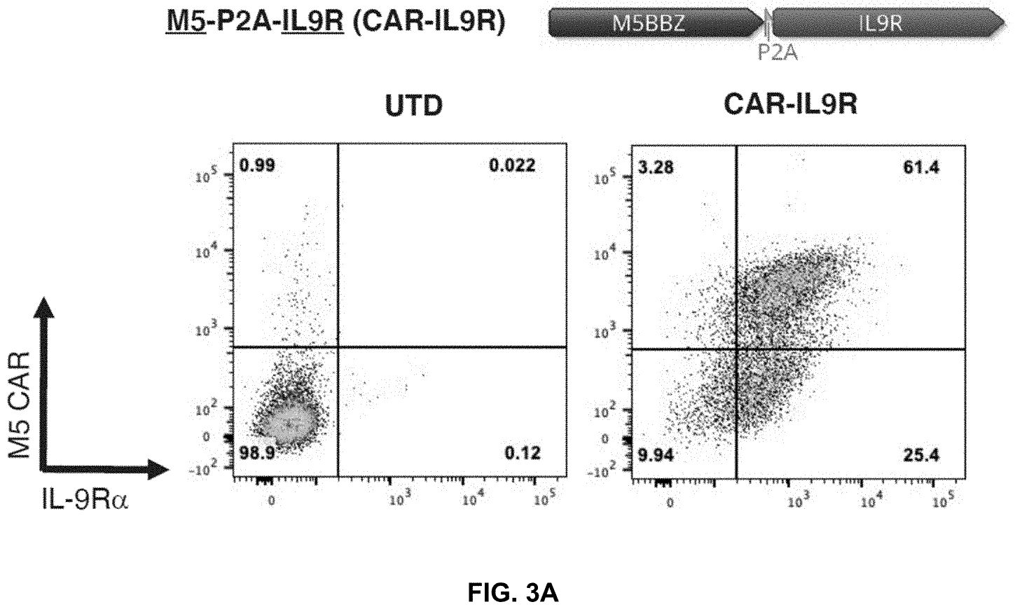

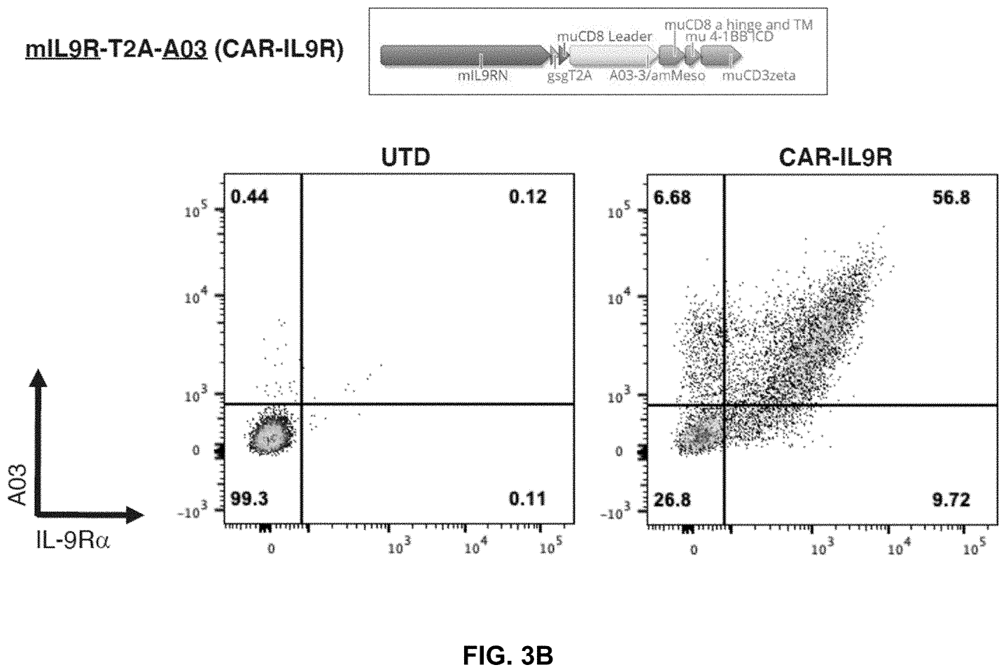

To demonstrate this, and to further investigate the mechanisms driving the unique phenotype and superior antitumor efficacy of CAR T cells engineered to receive an IL-9 signal, human and mouse lentiviral constructs for expression of WT IL9Ra and an anti-mesothelin CAR were designed and generated. Human and mouse T cells were transduced with their respective constructs, and co-expression of the CAR and IL9Ra was demonstrated by flow cytometry post transduction. FIG. 3A shows the data for the human T cells.

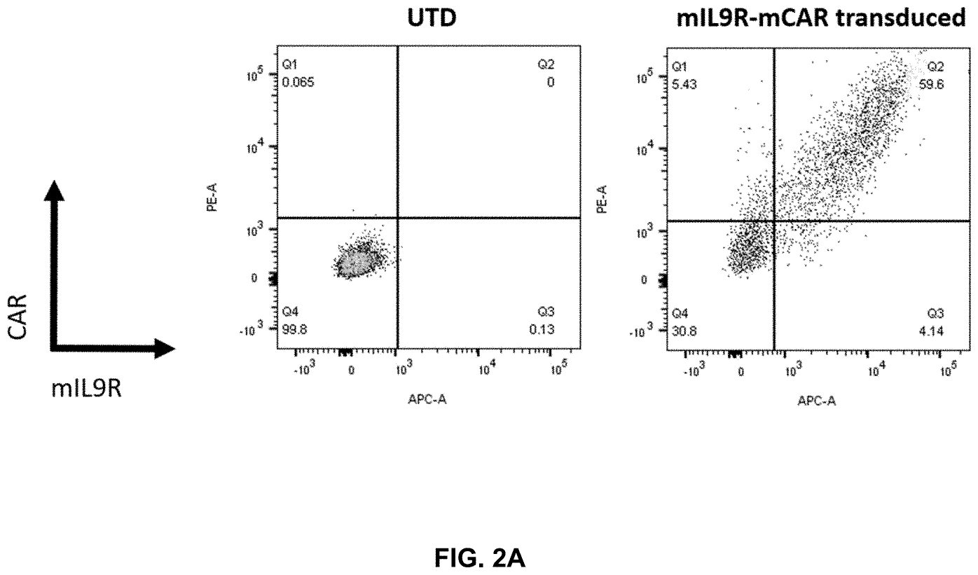

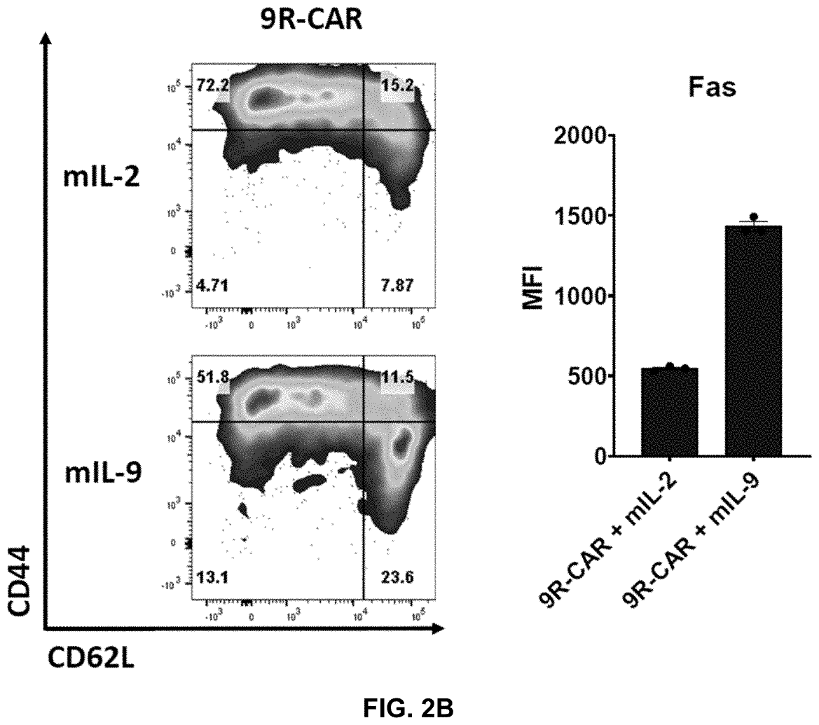

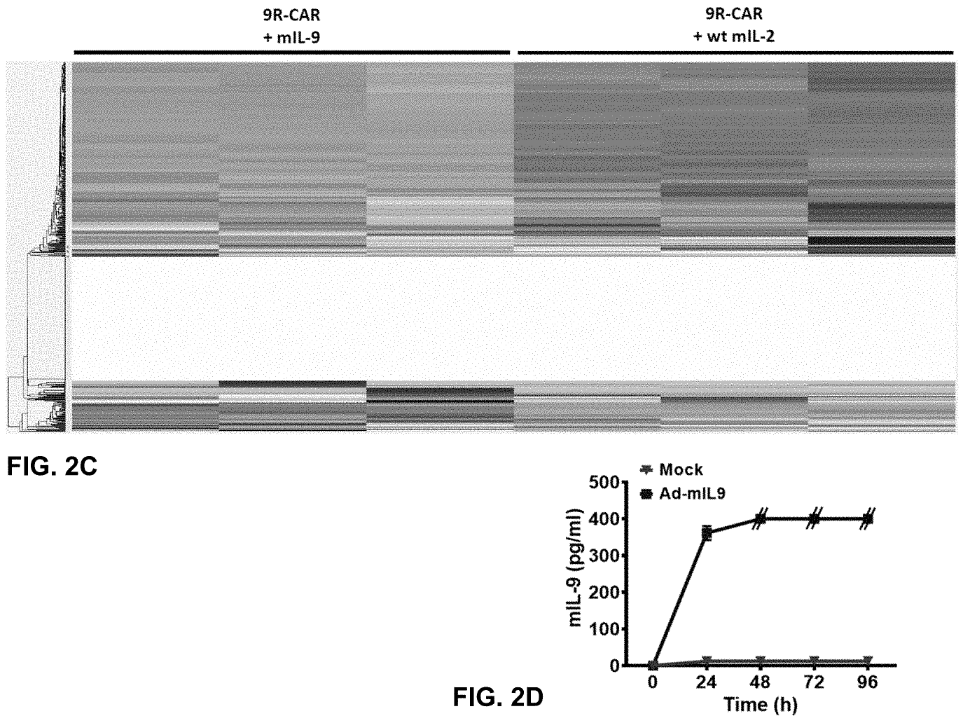

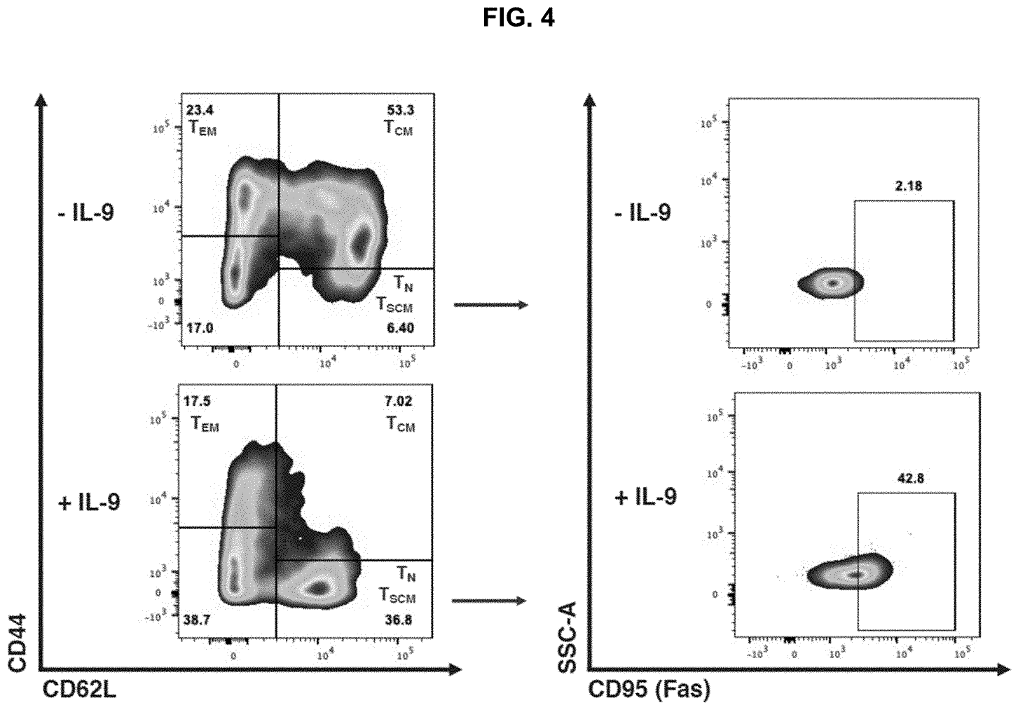

Co-expression of the mouse IL9Ra cytokine receptor and anti-mesothelin CAR on transduced mouse T cells was tested and demonstrated (FIG. 2A and FIG. 3B). Further, these cells demonstrated stem cell memory (Tscm) phenotype, as shown by expression of surface markers CD44, CD62L, and Fas (CD95) 24 h after stimulation with 100 ng/mL of wild type mIL9 or wild type mIL2 (FIG. 2B and FIG. 4). Next, total RNA was extracted from the transduced T cells cultured in the presence of mIL-2 or mIL-9 for 24 h. RNA was analyzed with Nanostring nCounter Mouse Immunology Panel (562 genes) and plotted using nSolver 4.0 software to obtain a global gene expression profile (FIG. 2C). These data further demonstate the Tscm phenotype of the transduced cells.

The transduced T cells co-expressing IL9Ra and a CAR comprising a tumor antigen binding domain will be used together with the oncolytic adenoviral vector expressing IL9 to treat cancer in a subject. As such, expression of IL9 from the Ad5 vector was tested and demonstrated in a murine pancreatic cancer cell line (FIG. 2D). Murine pancreatic cancer cell line PDA7940b (10,000 cells/well) was infected with 100 viral particles/cell of Ad-mIL9 and cell culture supernatants were analyzed for mIL-9 by ELISA at indicated time points.

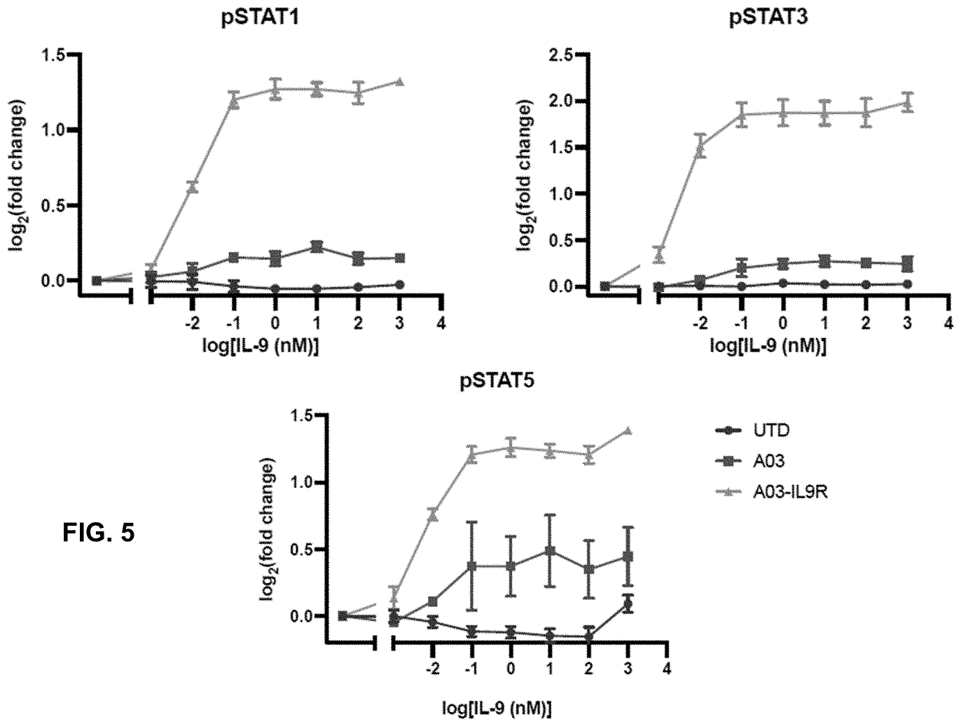

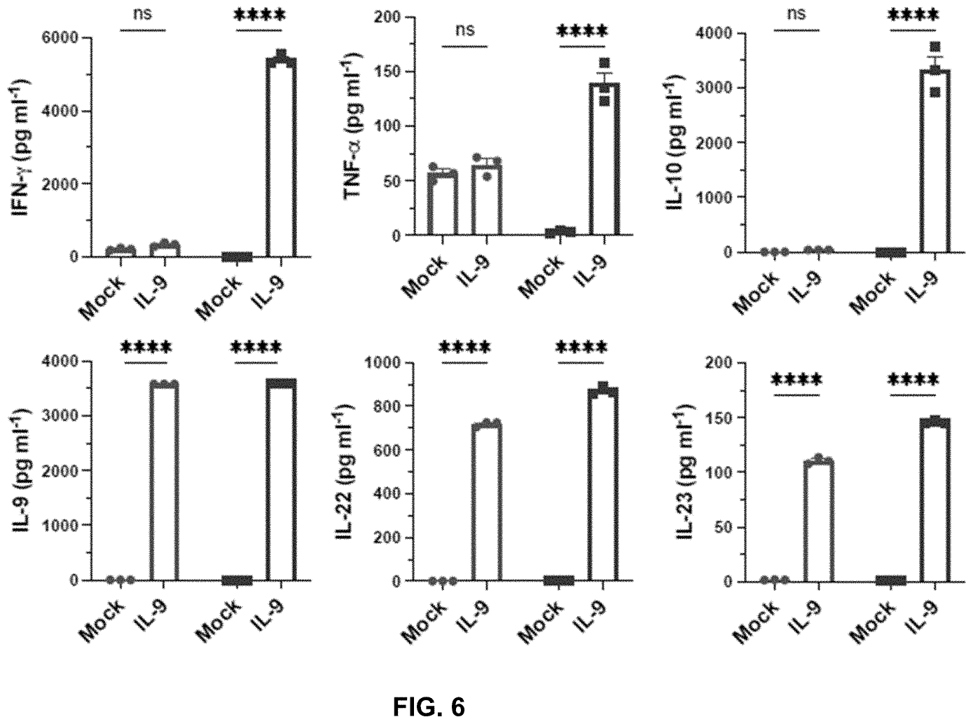

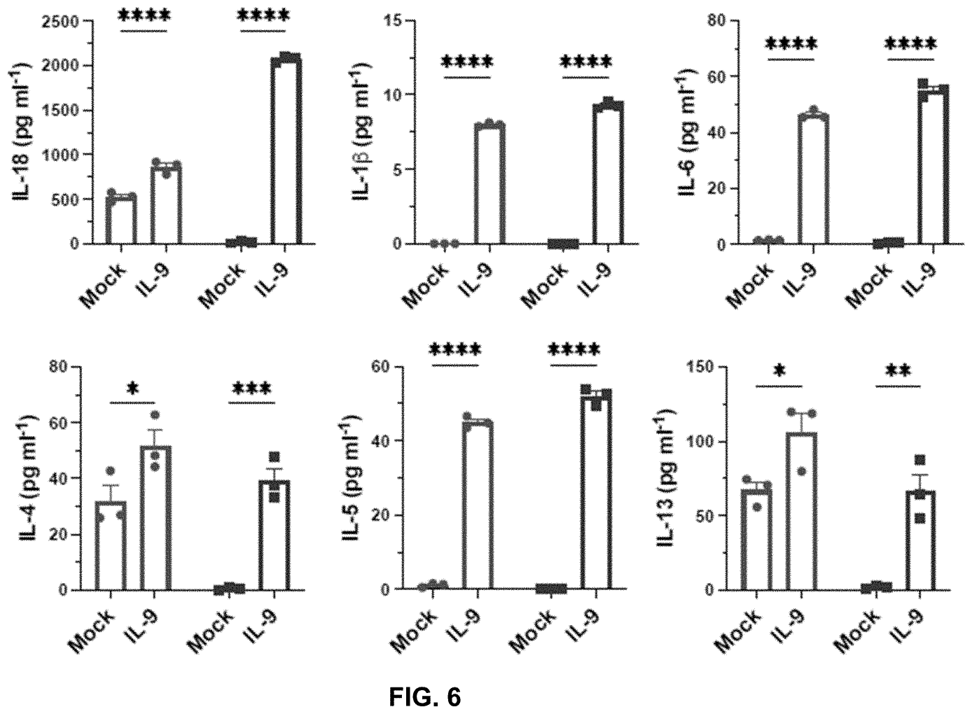

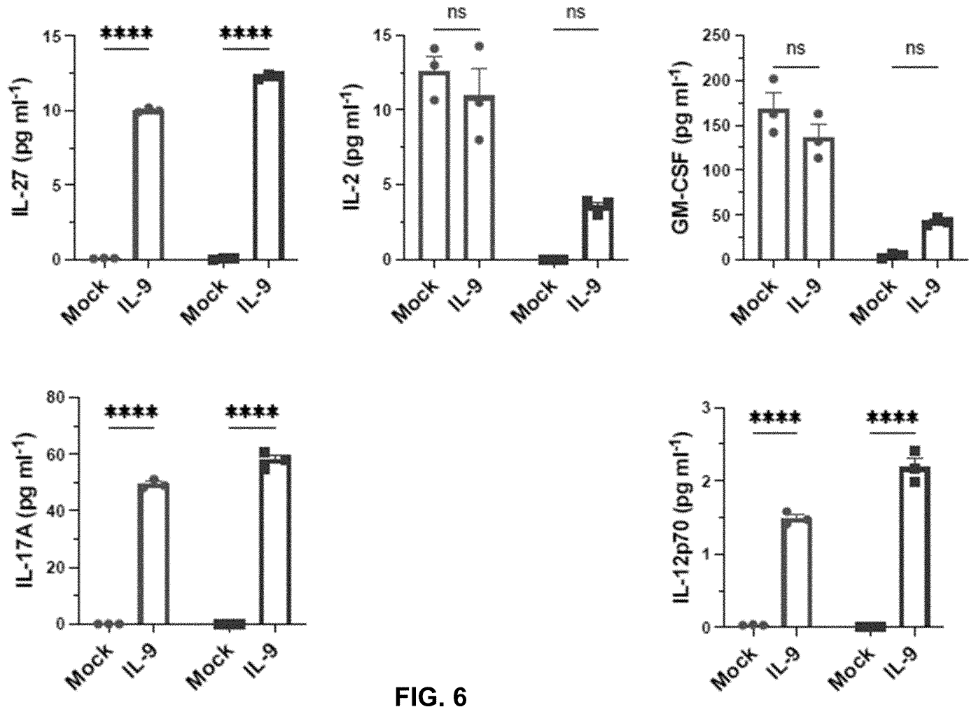

Next, the murine T cells were starved from IL-2 for 24 hours and then incubated in increasing concentrations of IL-9 for 20 minutes. Cells were fixed, permeabilized, stained, and analyzed by phosphor flow cytometry. Cells coexpressing IL9Ra and the CAR showed a significant increase in phosphorylation of STAT1, STAT3, and STAT5 compared to cells expressing only the CAR (A03) or untransduced cells (FIG. 5). Cytokine secretion was analyzed by Luminex assay after incubating the cells with IL9 for 48 hours. Co-expression of the CAR and IL9Ra significantly increased secretion of IFNγ, TNFα, IL-10, IL-18, IL-10, IL-6, IL-17A, IL-9, IL-22, IL-23, IL-4, IL-5, IL-13, IL-12p70, and IL-27 (FIG. 6, right side of each panel). For T cells expressing the CAR alone (without co-expression of IL9Ra), no significant increase was observed for IFNγ, TNFα, and IL-10, and the increases in IL-4 and IL-13 were less significant than that observed for cells co-expressing the CAR and IL9Ra (FIG. 6, left side of each panel).

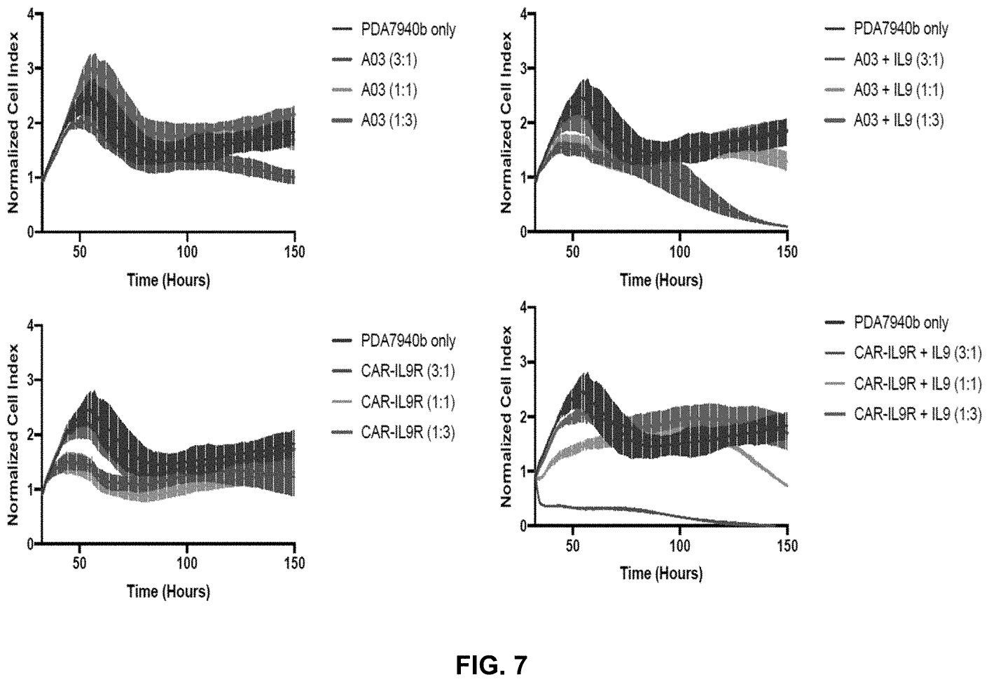

Next, tumor cell killing was assessed by seeding 5000 PDA7940b (pancreatic tumor) cells. Primary mouse T cells were pre-incubated for 48 hours with IL-9 (100 nM) (or no IL9, as a control) and added at T cells:tumor cells ratios of 3:1, 1:1, and 1:3. Tumor cell killing was significantly enhanced for T cells expressing both IL9Ra and anti-mesothelin CAR (A03) in the presence of IL9 (FIG. 7).

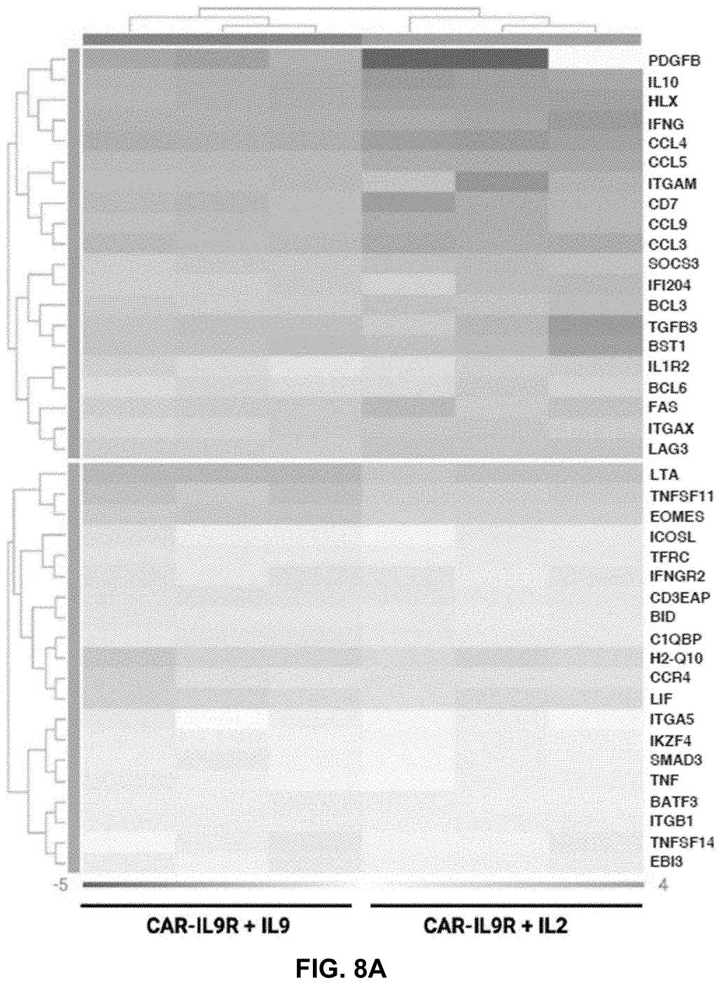

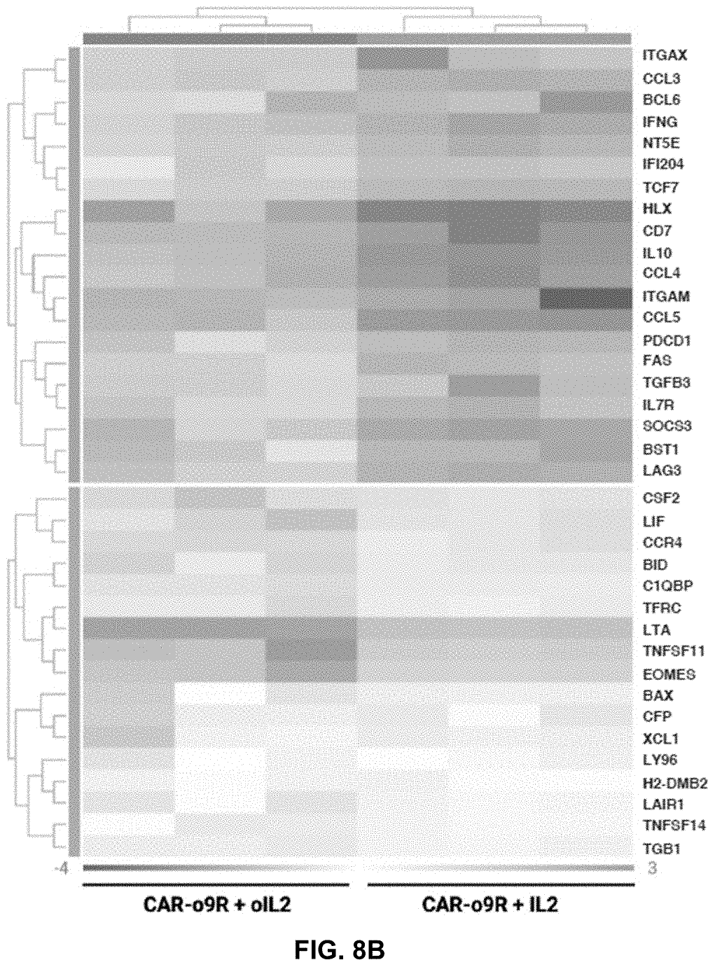

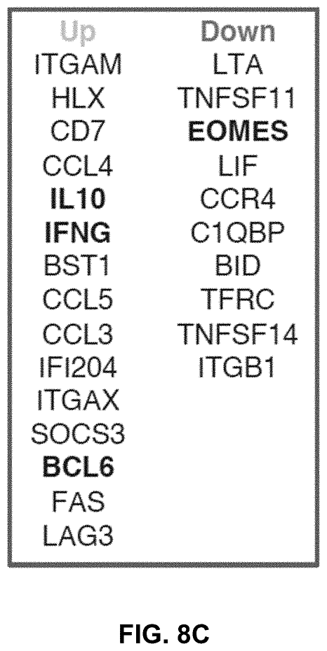

Gene expression profiles were determined with Nanostring nCounter Mouse Immunology Panel for the mouse T cells expressing the CAR and IL9Ra pre-incubated with IL9 (or IL2 as a control) (FIG. 8A). To compare with the orthogonal chimeric cytokine receptor system (see, WO 2021/050752; Kalbasi, et al., Nature, 2022, 607:360-365), gene expression profile was also determined for T cells expressing the CAR and an orthogonal chimeric cytokine receptor (ortho IL2Rβ-IL9Ra (“o9R”) pre-incubated with ortho-IL2 (or IL2 as a control) (FIG. 8B). The top 20 up-regulated and down-regulated genes for each are shown (p-adj<0.05) (FIGS. 8A-8B). Gene expression profiles were similar for the two. The shared top up-regulated and down-regulated genes are shown in FIG. 8C.

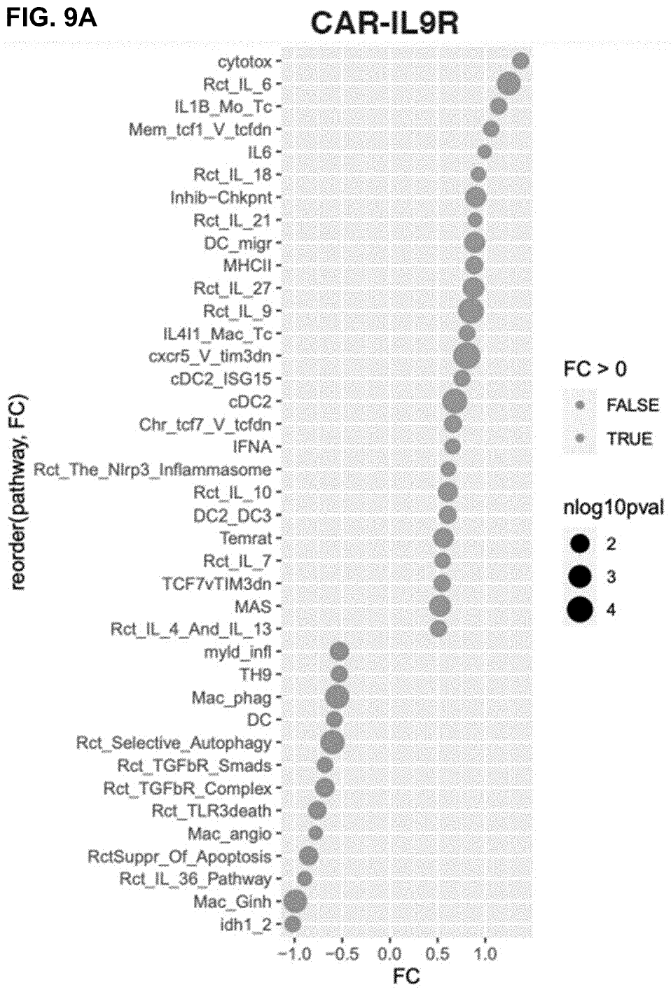

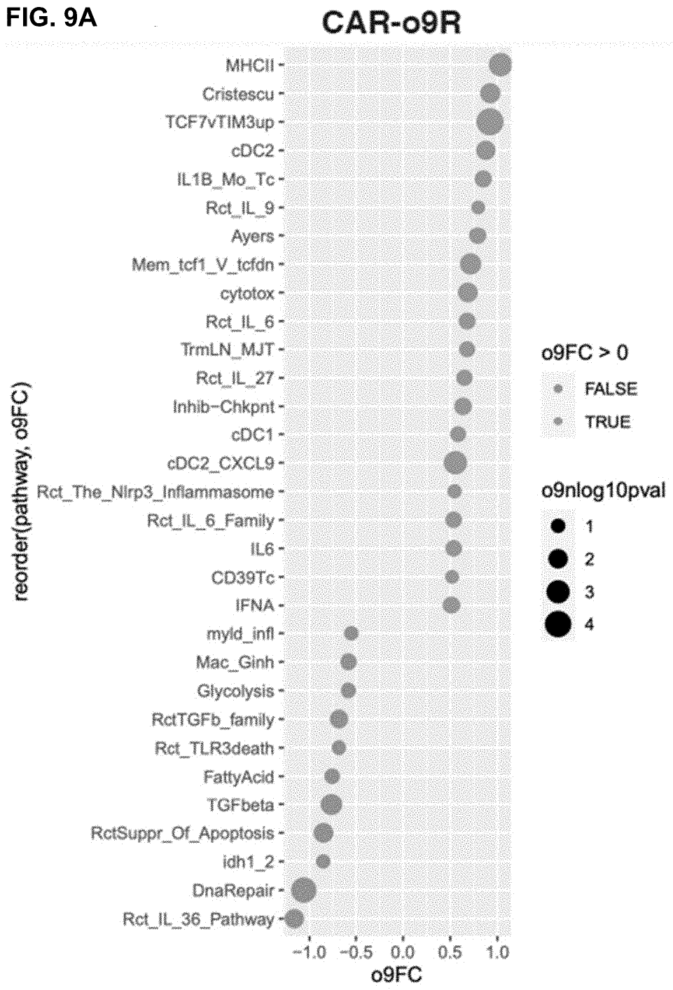

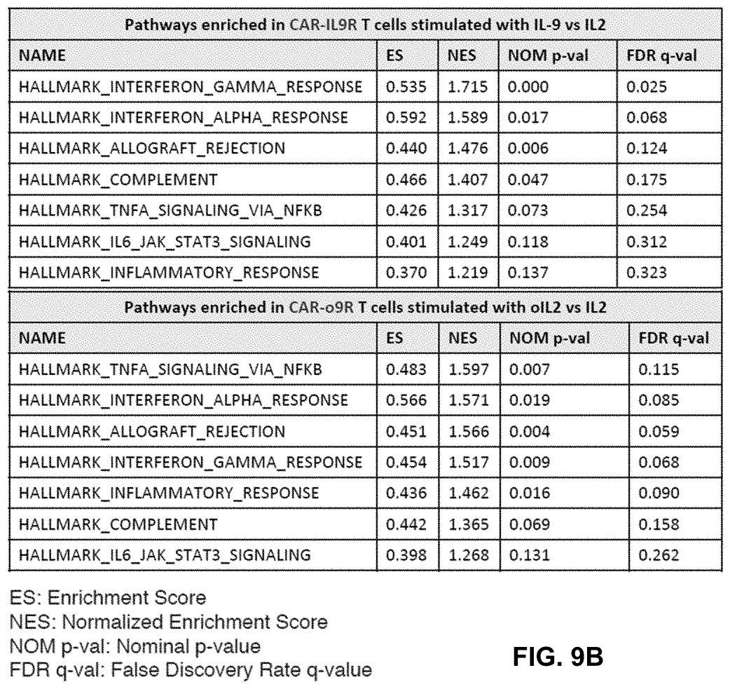

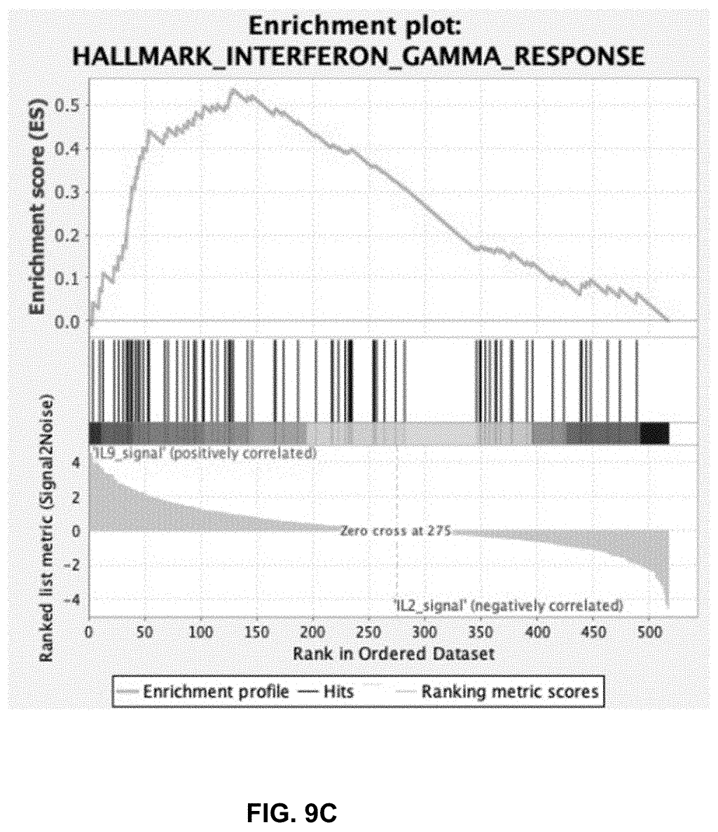

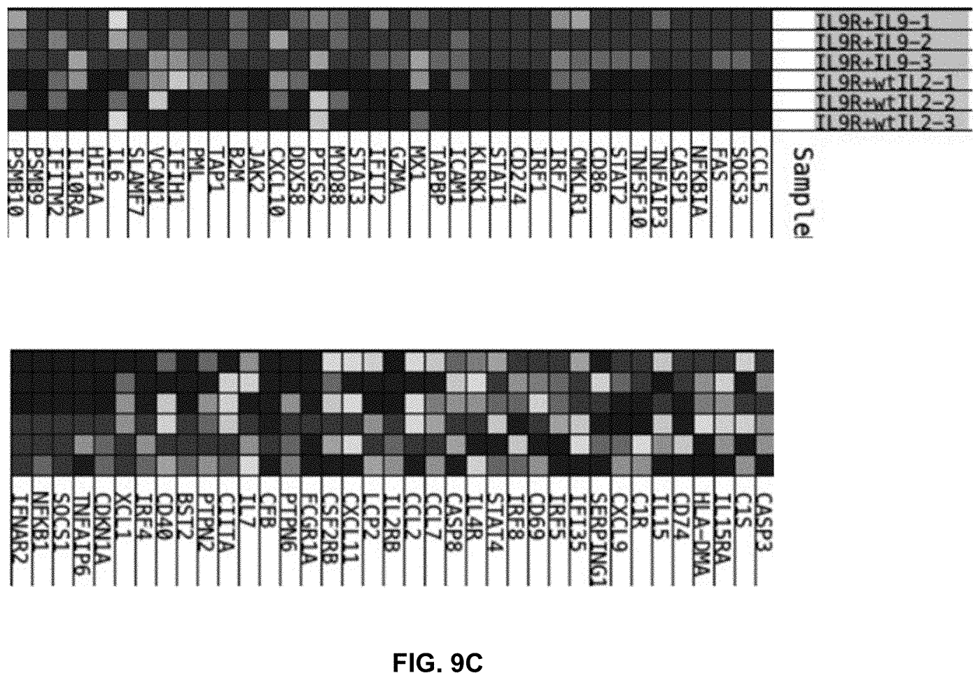

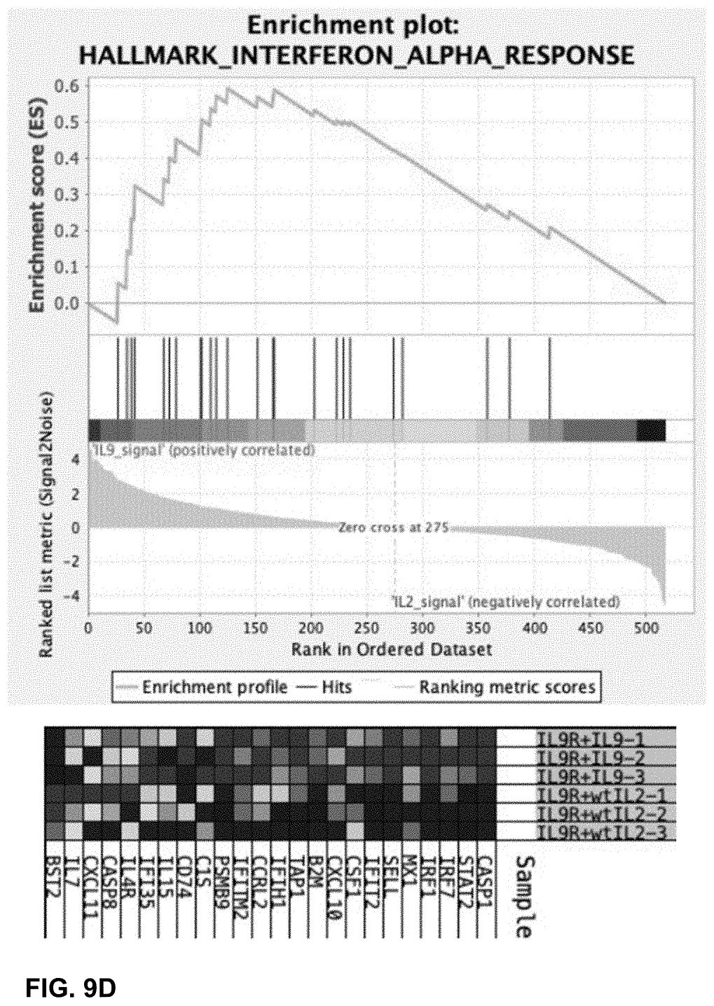

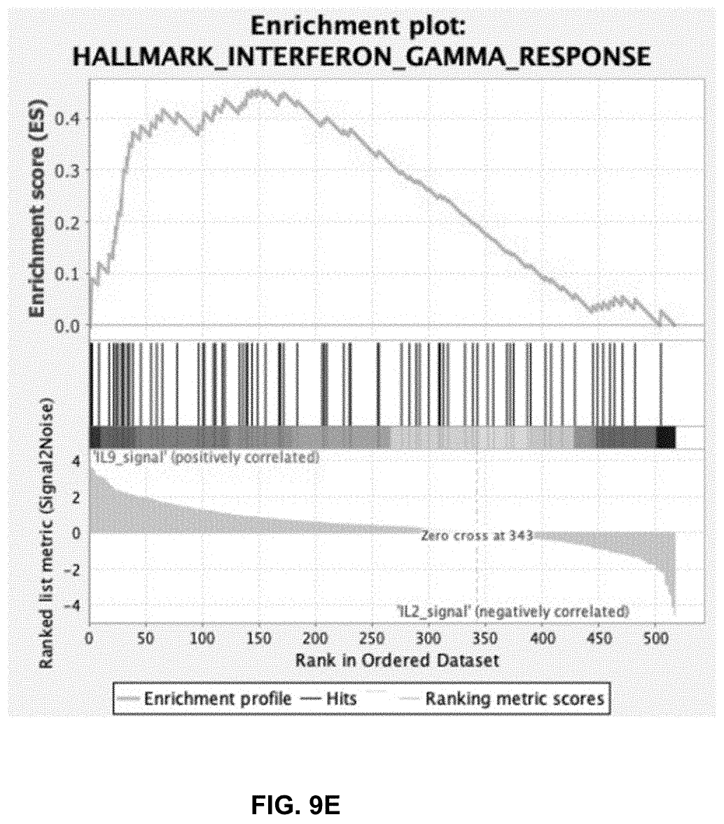

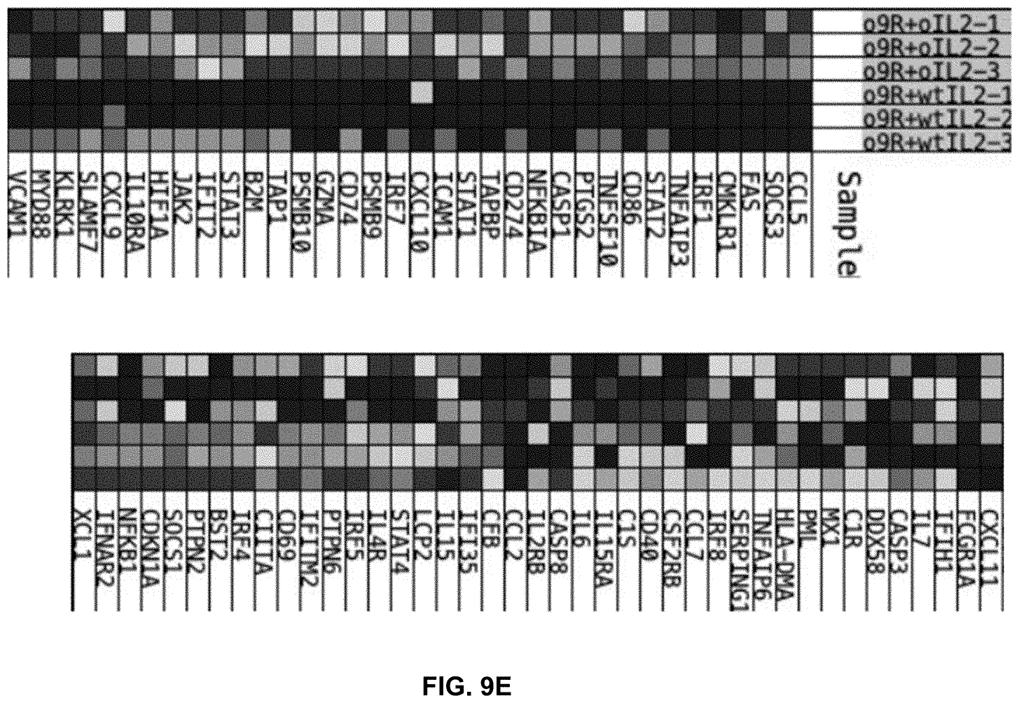

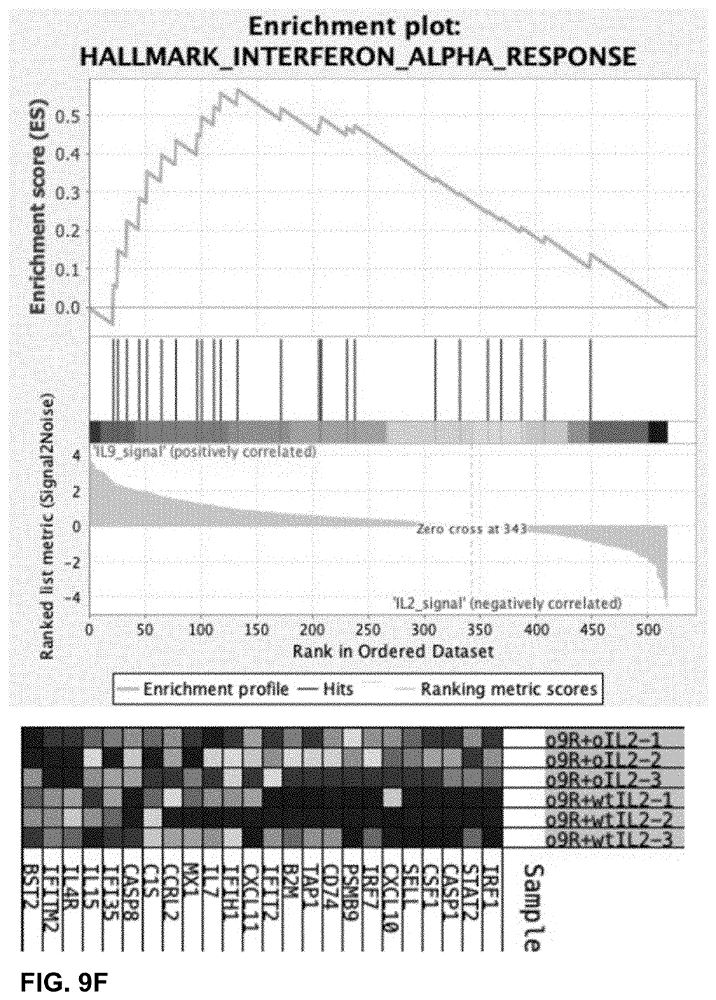

Next, gene set variation analysis (GSVA) was performed for T cells expressing anti-meso CAR and IL9Ra pre-incubated with either IL9 or IL2 and for T cells expressing anti-meso CAR and an ortho-IL2Rβ-IL9Ra chimeric cytokine receptor (o9R) pre-incubated with ortho-IL2 or IL-2. The pathways significantly enriched in the CAR T cells stimulated with IL9 vs IL2 are shown in FIGS. 9A-9C. Notably, the interferon gamma and interferon alpha pathways are significantly enriched in IL-9-stimulated CAR T cells (FIGS. 9D-9F).

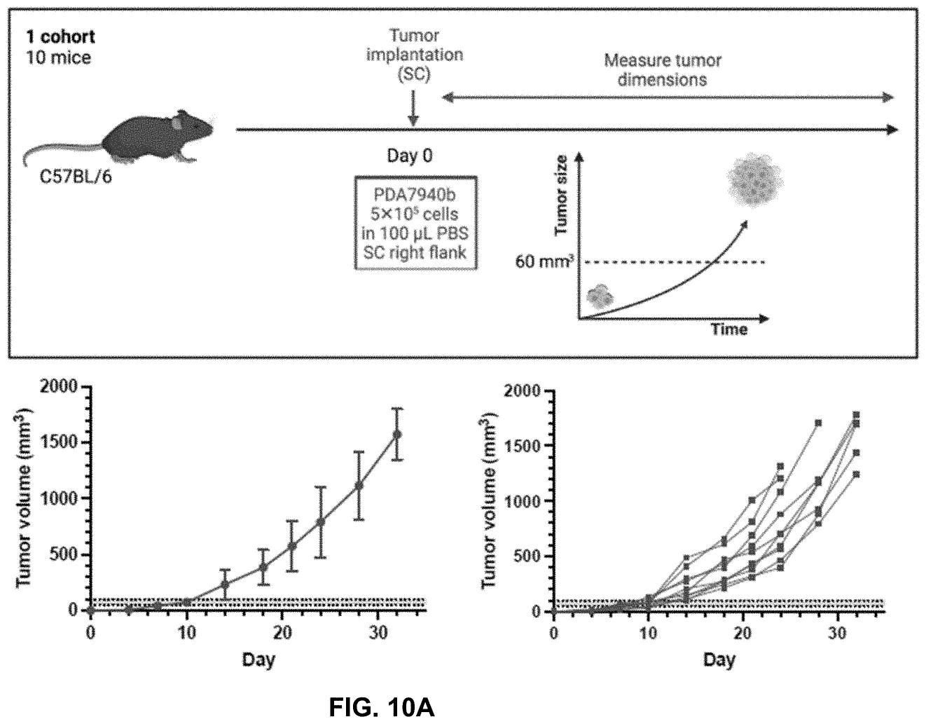

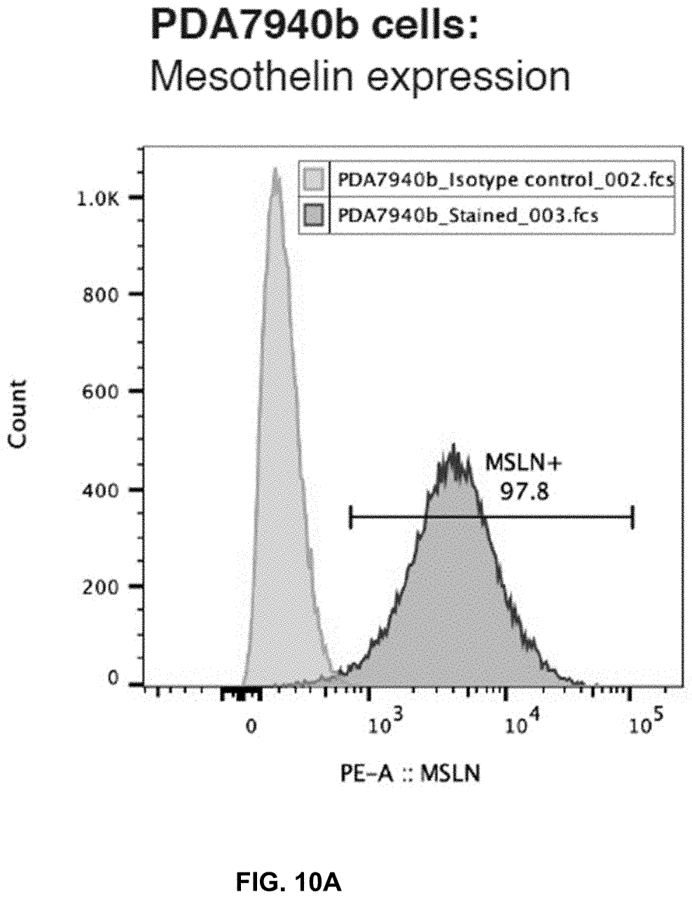

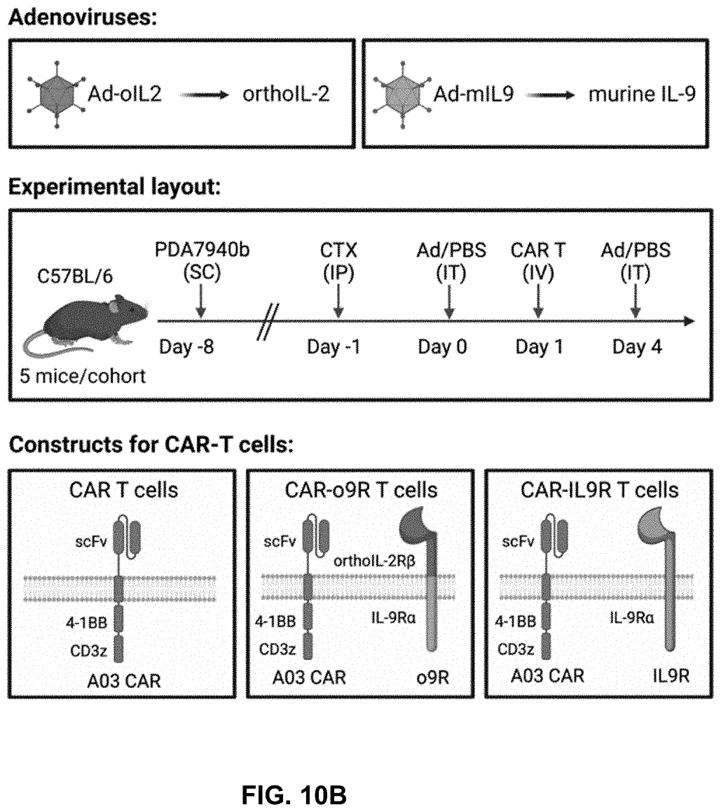

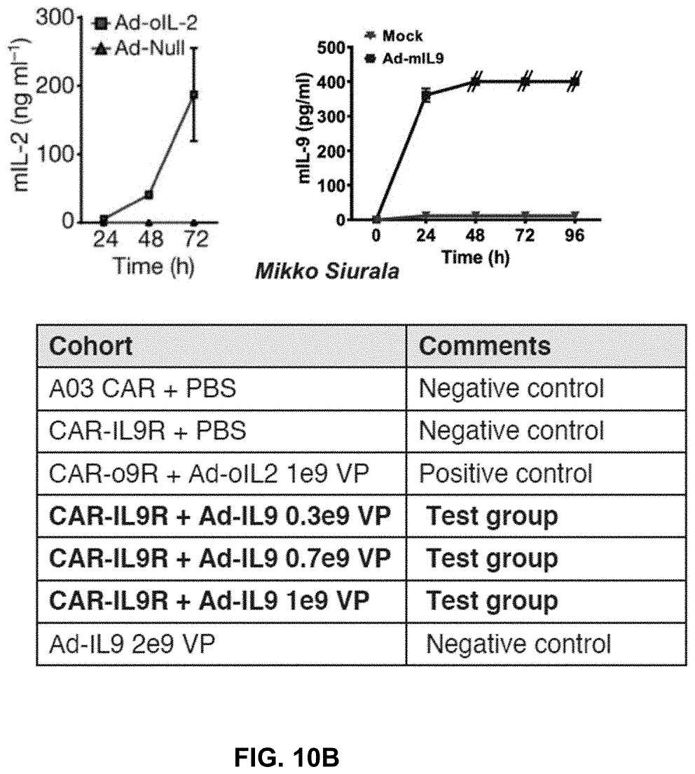

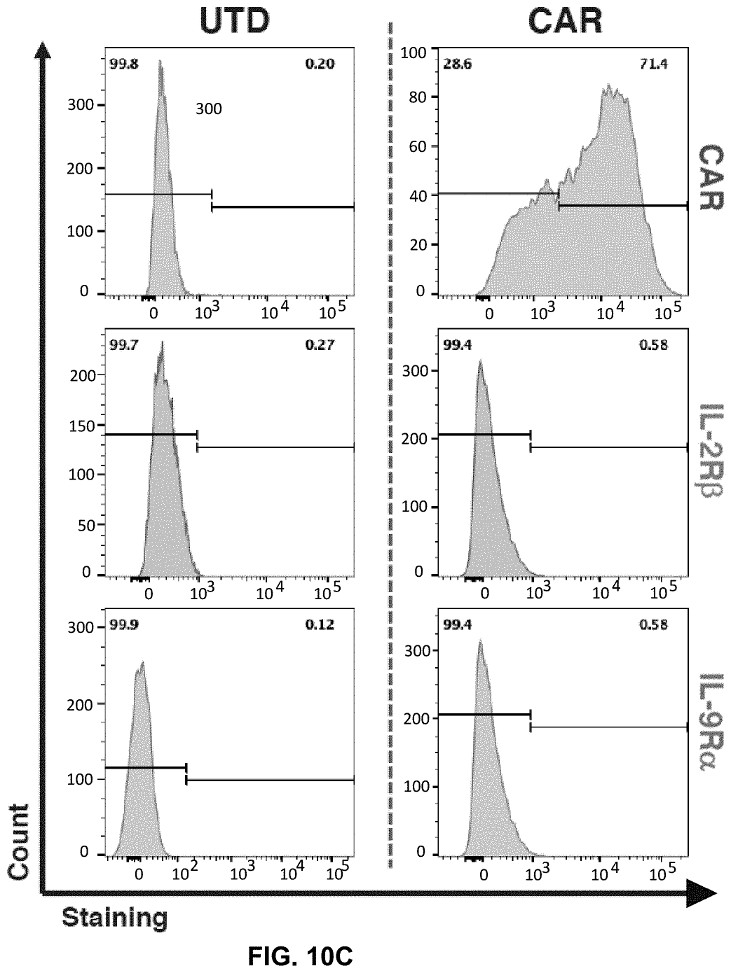

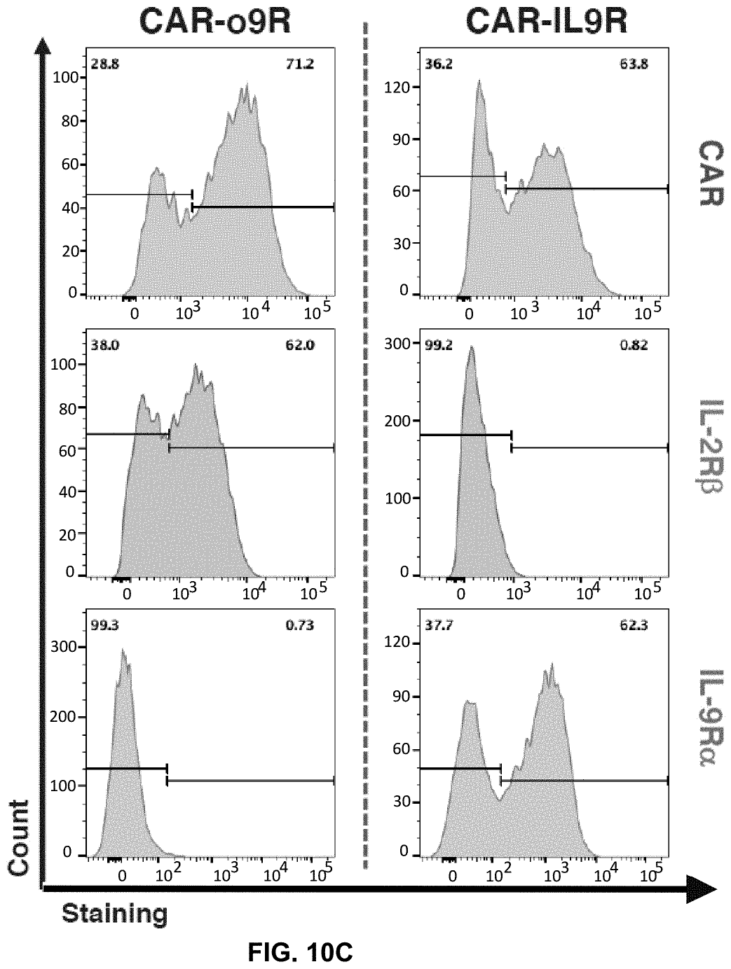

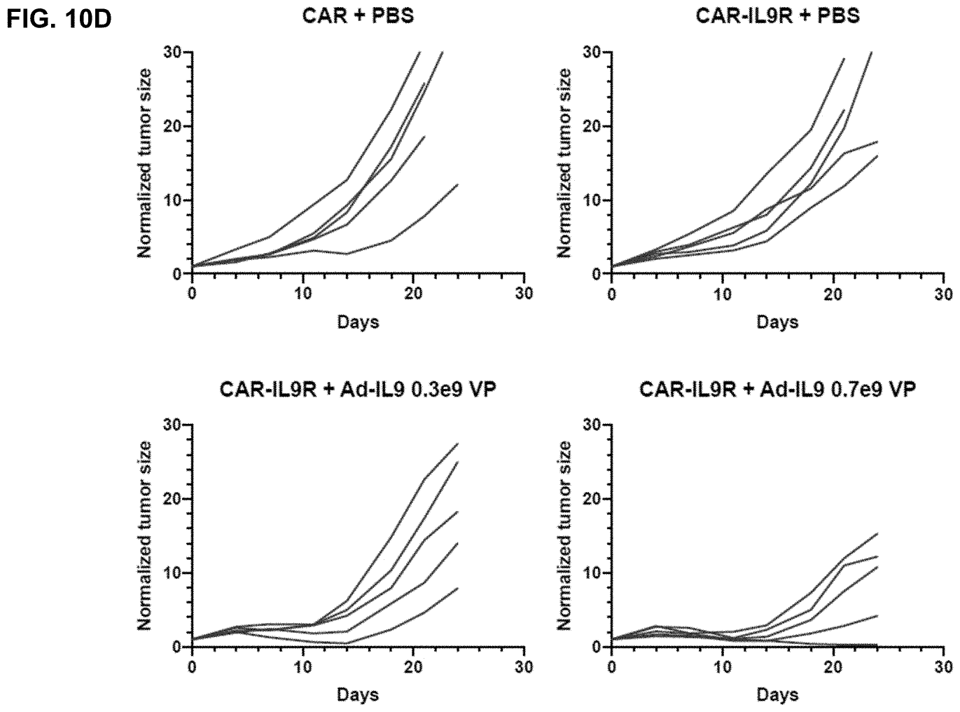

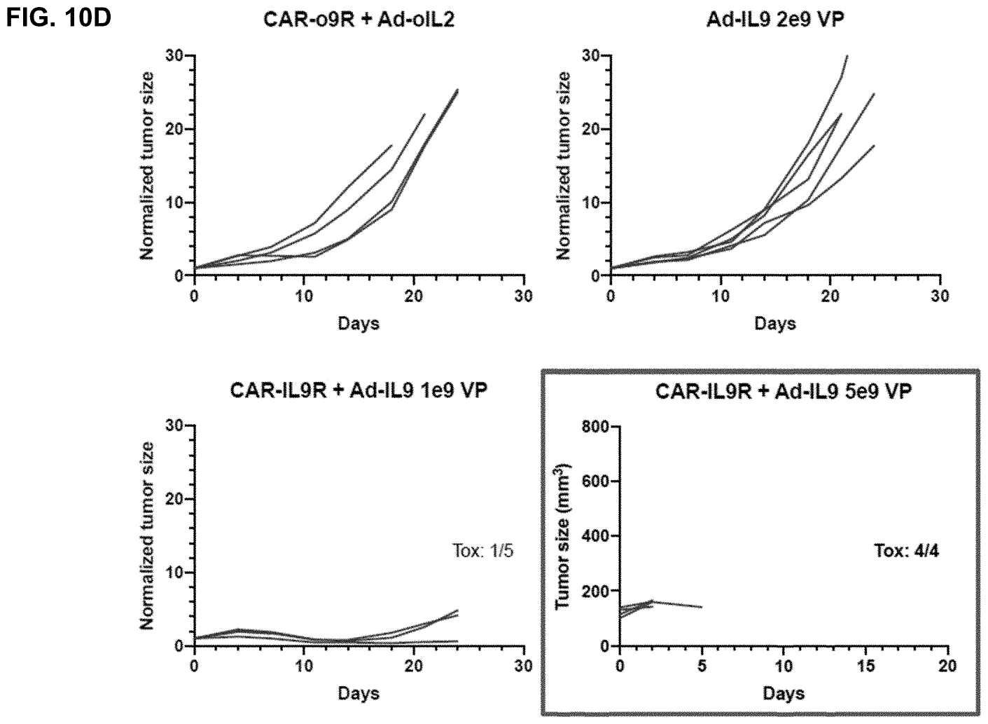

Next, an in vivo syngeneic murine model of PDA was established (FIG. 10A) and a dose titration for Ad vector expressing IL-9 (Ad-mIL9) was performed (FIG. 10B). Transduction efficiency data are shown in FIG. 10C. In vivo tumor growth was determined at various doses of Ad-mIL9 in mice injected with T cells expressing the CAR and IL9Ra, as well as the indicated control conditions (FIG. 10D).

### ENUMERATED EMBODIMENTS

The following enumerated embodiments are provided, the numbering of which is not to be construed as designating levels of importance.

Embodiment 1 provides a chimeric antigen receptor (CAR) comprising a tumor antigen binding domain, a transmembrane domain, and an intracellular domain comprising an intracellular signaling domain of an interleukin-9 receptor alpha (IL9Ra).

Embodiment 2 provides the CAR of embodiment 1, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

Embodiment 3 provides the CAR of embodiment 1 or embodiment 2, wherein the tumor antigen is selected from mesothelin, GD2, HER2, TnMuc1, CD70, PMSA, and EGFRvIII.

Embodiment 4 provides the CAR of any one of embodiments 1-3, wherein the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

Embodiment 5 provides the CAR of any one of embodiments 1-4, wherein the tumor antigen binding domain is a single-chain variable fragment (scFv).

Embodiment 6 provides the CAR of any one of embodiments 1-5, wherein the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

Embodiment 7 provides the CAR of any one of embodiments 1-6, wherein the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 8 provides the CAR of any one of embodiments 1-7, wherein the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 9 provides the CAR of any one of embodiments 1-8, wherein the intracellular domain of the CAR further comprises a costimulatory domain of a CD28, a costimulatory domain of a 4-1BB, an intracellular signaling domain of a CD3 zeta, or any combination thereof.

Embodiment 10 provides the CAR of any one of embodiments 1-9, further comprising a hinge domain.

Embodiment 11 provides the CAR of any one of embodiments 1-10, wherein the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

Embodiment 12 provides the CAR of any one of embodiments 1-11, wherein the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

Embodiment 13 provides the CAR of any one of embodiments 1-12, wherein the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

Embodiment 14 provides an isolated nucleic acid comprising a nucleotide sequence encoding a chimeric antigen receptor (CAR), wherein the CAR comprises a tumor antigen binding domain, a transmembrane domain, and an intracellular domain comprising an intracellular signaling domain of an interleukin-9 receptor alpha (IL9Ra).

Embodiment 15 provides the isolated nucleic acid of embodiment 14, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

Embodiment 16 provides the isolated nucleic acid of embodiment 14 or embodiment 15, wherein the tumor antigen is selected from mesothelin, GD2, HER2, TnMuc1, CD70, PMSA, and EGFRvIII.

Embodiment 17 provides the isolated nucleic acid of any one of embodiments 14-16, wherein the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

Embodiment 18 provides the isolated nucleic acid of any one of embodiments 14-17, wherein the tumor antigen binding domain is a single-chain variable fragment (scFv).

Embodiment 19 provides the isolated nucleic acid of any one of embodiments 14-18, wherein the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

Embodiment 20 provides the isolated nucleic acid of any one of embodiments 14-19, wherein the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 21 provides the isolated nucleic acid of any one of embodiments 14-20, wherein the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 22 provides the isolated nucleic acid of any one of embodiments 14-21, wherein the intracellular domain of the CAR further comprises a costimulatory domain of a CD28, a costimulatory domain of a 4-1BB, an intracellular signaling domain of a CD3 zeta, or any combination thereof.

Embodiment 23 provides the isolated nucleic acid of any one of embodiments 14-22, further comprising a hinge domain.

Embodiment 24 provides the isolated nucleic acid of any one of embodiments 14-23, wherein the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

Embodiment 25 provides the isolated nucleic acid of any one of embodiments 14-24, wherein the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

Embodiment 26 provides the isolated nucleic acid of any one of embodiments 14-25, wherein the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

Embodiment 27 provides a vector comprising the isolated nucleic acid of any one of embodiments 14-26.

Embodiment 28 provides the vector of embodiment 27, wherein the vector is a retroviral vector or a lentiviral vector.

Embodiment 29 provides a modified cell, wherein the cell is an immune cell or precursor cell thereof, and wherein the cell is engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

Embodiment 30 provides the modified cell of embodiment 29, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

Embodiment 31 provides the modified cell of embodiment 29 or embodiment 30, wherein the tumor antigen is selected from mesothelin, GD2, HER2, TnMuc1, CD70, PMSA, and EGFRvIII.

Embodiment 32 provides the modified cell of any one of embodiments 29-31, wherein the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

Embodiment 33 provides the modified cell of any one of embodiments 29-32, wherein the tumor antigen binding domain is a single-chain variable fragment (scFv).

Embodiment 34 provides the modified cell of any one of embodiments 29-33, wherein the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

Embodiment 35 provides the modified cell of any one of embodiments 29-34, wherein the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 36 provides the modified cell of any one of embodiments 29-35, wherein the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 37 provides the modified cell of any one of embodiments 29-36, wherein the intracellular domain of the CAR further comprises a costimulatory domain of a CD28, a costimulatory domain of a 4-1BB, an intracellular signaling domain of a CD3 zeta, or any combination thereof.

Embodiment 38 provides the modified cell of any one of embodiments 29-37, further comprising a hinge domain.

Embodiment 39 provides the modified cell of any one of embodiments 29-38, wherein the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

Embodiment 40 provides the modified cell of any one of embodiments 29-39, wherein the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

Embodiment 41 provides the modified cell of any one of embodiments 29-40, wherein the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

Embodiment 42 provides the modified cell of any one of embodiments 29-41, wherein the cell is a T cell, an autologous cell, a human cell, or any combination thereof.

Embodiment 43 provides the modified cell of any one of embodiments 29-42, wherein the cell is capable of activating STAT1, STAT3, STAT5, or any combination thereof.

Embodiment 44 provides a pharmaceutical composition comprising a population of the modified cell of any one of embodiments 29-43 and at least one pharmaceutically acceptable carrier.

Embodiment 45 provides a method of treating cancer in a subject in need thereof, comprising administering to the subject a population of modified cells, wherein the cells are immune cells or precursor cells thereof, and wherein the cells are engineered to express a chimeric antigen receptor (CAR) comprising a tumor antigen binding domain, a transmembrane domain, and an intracellular domain comprising an intracellular signaling domain of an interleukin-9 receptor alpha (IL9Ra).

Embodiment 46 provides the method of embodiment 45, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

Embodiment 47 provides the method of embodiment 45 or embodiment 46, wherein the tumor antigen is selected from mesothelin, GD2, HER2, TnMuc1, CD70, PMSA, and EGFRvIII.

Embodiment 48 provides the method of any one of embodiments 45-47, wherein the tumor antigen binding domain is selected from the group consisting of a full length antibody or antigen-binding fragment thereof, a monospecific antibody, a bispecic antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

Embodiment 49 provides the method of any one of embodiments 45-48, wherein the tumor antigen binding domain is a single-chain variable fragment (scFv).

Embodiment 50 provides the method of any one of embodiments 45-49, wherein the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

Embodiment 51 provides the method of any one of embodiments 45-50, wherein the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 52 provides the method of any one of embodiments 45-51, wherein the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 53 provides the method of any one of embodiments 45-52, wherein the intracellular domain of the CAR further comprises a costimulatory domain of a CD28, a costimulatory domain of a 4-1BB, an intracellular signaling domain of a CD3 zeta, or any combination thereof.

Embodiment 54 provides the method of any one of embodiments 45-53, further comprising a hinge domain.

Embodiment 55 provides the method of any one of embodiments 45-54, wherein the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

Embodiment 56 provides the method of any one of embodiments 45-55, wherein the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

Embodiment 57 provides the method of any one of embodiments 45-56, wherein the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

Embodiment 58 provides the method of any one of embodiments 45-57, wherein the population of cells comprises T cells, autologous cells, human cells, or any combination thereof.

Embodiment 59 provides the method of any one of embodiments 45-58, wherein the population of cells is capable of activating STAT1, STAT3, STAT5, or any combination thereof.

Embodiment 60 provides the method of any one of embodiments 45-59, wherein the subject is a human.

Embodiment 61 provides the method of any one of embodiments 45-60, wherein the cancer is selected from a B-cell malignancy (such as a B-cell lymphomas or leukemia), lung cancer, non-small cell lung cancer, small cell lung cancer, Merkel cell carcinoma, melanoma, Hodgkin's lymphoma, non-Hodgkin's lymphoma, urothelial carcinoma, gastric cancer, cervical cancer, cutaneous squamous cell carcinoma, renal cell carcinoma, breast cancer, triple-negative breast cancer, colon cancer, esophagus cancer, stomach cancer, liver cancer, kidney cancer, pancreatic cancer, prostate cancer, brain cancer, lung adenocarcinoma, glioblastoma, hepatocellular carcinoma, gallbladder cancer, cervical cancer, cervical squamous cell carcinoma, colorectal cancer, ovarian cancer, and renal cancer.

### OTHER EMBODIMENTS

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A chimeric antigen receptor (CAR) comprising a tumor antigen binding domain, a transmembrane domain, and an intracellular domain comprising an intracellular signaling domain of an interleukin-9 receptor alpha (IL9Ra).

2. The CAR of claim 1, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-2 (GPC2), glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, TnMuc1, VEGFR2, and any combination thereof.

3. The CAR of claim 1, wherein the tumor antigen is selected from mesothelin, GD2, HER2, TnMuc1, CD70, PMSA, and EGFRvIII.

4. (canceled)

5. The CAR of claim 1, wherein the tumor antigen binding domain is a single-chain variable fragment (scFv).

6. The CAR of claim 1, wherein the tumor antigen binding domain is selected from:
(a) an anti-mesothelin scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NO: 49 and SEQ ID NO: 65;
(b) an anti-GD2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 108;
(c) an anti-HER2 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 110 or SEQ ID NO: 112;
(d) an anti-TnMuc1 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 114;
(e) an anti-CD70 scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 116;
(f) an anti-PMSA scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 120; and
(g) an anti-EGFRvIII scFv comprising an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to SEQ ID NO: 122.

7. The CAR of claim 1, wherein the intracellular domain of the CAR further comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

8. The CAR of claim 7, wherein the intracellular domain of the CAR further comprises an intracellular signaling domain of a protein selected from the group consisting of a CD3 zeta chain (CD3ζ), FcTRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

9. The CAR of claim 1, wherein the intracellular domain of the CAR further comprises a costimulatory domain of a CD28, a costimulatory domain of a 4-1BB, an intracellular signaling domain of a CD3 zeta, or any combination thereof.

10. The CAR of claim 1, further comprising a hinge domain.

11. The CAR of claim 1, wherein the CAR comprises:
(a) an anti-human mesothelin scFv, a human CD8 hinge domain, a human CD28 transmembrane domain, a human CD28 costimulatory domain, a human IL9Ra intracellular signaling domain, and a human CD3z signaling domain;
(b) an anti-human mesothelin scFv, a human CD8 hinge domain, a human CD8 transmembrane domain, a human 4-1BB costimulatory domain, a human IL9Ra intracellular signaling domain, and a human CD3z signaling domain;
(c) an anti-murine mesothelin scFv, a murine CD8 hinge domain, a murine CD28 transmembrane domain, a murine CD28 costimulatory domain, a murine IL9Ra intracellular signaling domain, and a murine CD3z signaling domain; or
(d) an anti-murine mesothelin scFv, a murine CD8 hinge domain, a murine CD8 transmembrane domain, a murine 4-1BB costimulatory domain, a murine IL9Ra intracellular signaling domain, and a murine CD3z signaling domain.

12. The CAR of claim 1, wherein the CAR comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 81, 83, 85, and 87.

13. The CAR of claim 1, wherein the CAR is encoded by a nucleic acid comprising a nucleotide sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to a sequence selected from SEQ ID NOs: 82, 84, 86, and 88.

14. An isolated nucleic acid comprising a nucleotide sequence encoding the CAR of claim 1.

15-26. (canceled)

27. A vector comprising the isolated nucleic acid of claim 14.

28. The vector of claim 27, wherein the vector is a retroviral vector or a lentiviral vector.

29. A modified cell, wherein the cell is an immune cell or precursor cell thereof, and wherein the cell is engineered to express the CAR of claim 1.

30-41. (canceled)

42. The modified cell of claim 29, wherein the cell is a T cell, an autologous cell, a human cell, or any combination thereof.

43. The modified cell of claim 29, wherein the cell is capable of activating STAT1, STAT3, STAT5, or any combination thereof.

44. A pharmaceutical composition comprising a population of the modified cell of claim 29 and at least one pharmaceutically acceptable carrier.

45. A method of treating cancer in a subject in need thereof, comprising administering to the subject a population of the modified cell of claim 29.

46-59. (canceled)

60. The method of claim 45, wherein the subject is a human.

61. The method of claim 45, wherein the cancer is selected from a B-cell malignancy (such as a B-cell lymphomas or leukemia), lung cancer, non-small cell lung cancer, small cell lung cancer, Merkel cell carcinoma, melanoma, Hodgkin's lymphoma, non-Hodgkin's lymphoma, urothelial carcinoma, gastric cancer, cervical cancer, cutaneous squamous cell carcinoma, renal cell carcinoma, breast cancer, triple-negative breast cancer, colon cancer, esophagus cancer, stomach cancer, liver cancer, kidney cancer, pancreatic cancer, prostate cancer, brain cancer, lung adenocarcinoma, glioblastoma, hepatocellular carcinoma, gallbladder cancer, cervical cancer, cervical squamous cell carcinoma, colorectal cancer, ovarian cancer, and renal cancer.
